(19) United States
(12) Patent Application Publication
(10) Pub. No.: US 2004/0265315 A1
(43) Pub. Date: Dec. 30, 2004

(54) Methods of preventing or treating T cell malignancies by administering CD2 antagonists

(76) Inventors: Christine Dingivan, Gaithersburg, MD (US); Thomas Waldmann, Silver Spring, MD (US); Zhuo Zhang, Clarksburg, MD (US); Meill Zhang, Bethesda, MD (US)

Correspondence Address: JONES DAY, 222 EAST 41ST ST, NEW YORK, NY, 10017, US

(21) Appl. No.: 10/657,006

(22) Filed: Sep. 5, 2003

Related U.S. Application Data
(60) Provisional application No. 60/409,024, filed on Sep. 5, 2002.
(60) Provisional application No. 60/410,385, filed on Sep. 12, 2002.

(51) Int. Cl. A61K 39/395

(52) U.S. Cl. 424/155.1

(57) ABSTRACT

The present invention encompasses the use of a CD2 antagonist, preferably MEDI-507, an analog, derivative or an antigen-binding fragment thereof as a single agent therapy for the prevention, treatment, management, or amelioration of cancer, particularly a T-cell malignancy, or one or more symptoms thereof. The present invention also encompasses the use of a CD2 antagonist, preferably MEDI-507, an analog, derivative or an antigen-binding fragment thereof in combination with other cancer therapies. The present invention provides pharmaceutical compositions comprising a CD2 antagonist, preferably MEDI-507, an analog, derivative or an antigen-binding fragment thereof in amounts effective to prevent, treat, manage, or ameliorate cancer, particularly a T-cell malignancy, or one or more symptoms thereof

This application claims priority to U.S. Provisional Patent Application No. 60/409,024 filed on Sep. 5, 2002 and U.S. Provisional Patent Application No. 60/410,385 filed on Sep. 12, 2002, each of which is incorporated herein by reference in its entirety.

### 1. FIELD OF THE INVENTION

The present invention encompasses the use of a CD2 antagonist, preferably MEDI-507, an analog, derivative or an antigen-binding fragment thereof as a single agent therapy for the treatment, prevention, management, or amelioration of cancer, a particularly T-cell malignancy, or one or more symptoms thereof The present invention also encompasses the use of a CD2 antagonist, preferably MEDI-507, an analog, derivative or an antigen-binding fragment thereof in combination with other cancer therapies. The present invention provides pharmaceutical compositions comprising a CD2 antagonist, preferably MEDI-507, an analog, derivative or an antigen-binding fragment thereof in amounts effective to prevent, treat, manage, or ameliorate cancer, particularly a T-cell malignancy, or one or more symptoms thereof.

### 2. BACKGROUND OF THE INVENTION

### 2.1 Cancer

A neoplasm or tumor is a neoplastic mass resulting from abnormal uncontrolled cell growth which can be benign or malignant. Benign tumors generally remain localized. Malignant tumors are collectively termed cancers. The term “malignant” generally means that the tumor can invade and destroy neighboring body structures and spread to distant sites to cause death (for review, see Robbins and Angell, 1976, Basic Pathology, 2d Ed., W.B. Saunders Co., Philadelphia, pp. 68-122). Cancer can arise in many sites of the body and behave differently depending upon its origin. Cancerous cells destroy the part of the body in which they originate and then spread to other part(s) of the body where they start new growth and cause more destruction.

More than 1.2 million Americans develop cancer each year. Cancer is the second leading cause of death in the United States and if current trends continue, cancer is expected to be the leading cause of the death by the year 2010. Lung cancer and prostate cancer are the top cancer killers for men in the United States. Lung cancer and breast cancer are the top cancer killers for women in the United States. One in two men in the United States will be diagnosed with cancer at some time during his lifetime. One in three women in the United States will be diagnosed with cancer at some time during her lifetime.

### 2.2 T-Cell Malignancies

Tumors of T-cell origin and other cells involved in T-cell development have been identified. T-cell lymphoproliferative disorders include thymic and post-thymic malignancies. T-cell neoplasms include tumors of lymphoid progenitor cells, thymic stromal or epithelial cells, thymocytes, T-cells, natural killer (“NK”) cells, or antigen-presenting cells. T-cell malignancies include acute lymphoblastic leukemias, lymphomas, thymomas, acute lymphoblastic leukemias, Hodgkin's and non-Hodgkin's disease. Lymphomas are categorized by how the T-cells are affected. A more in-depth list of lymphoma classifications and types is available for reference and is summarized in Table 1, infra. T-cell lymphomas include, for example, lymphoblastic lymphoma, anaplastic large cell lymphoma, peripheral T-cell lymphomas, angioimmunoblastic lymphoma, angiocentric lymphoma (nasal T-cell lymphoma), intestinal T-cell lymphoma, and adult T-cell lymphoma/leukemia, some of which are discussed below.

Lymphoblastic Lymphoma

Lymphoblastic lymphoma is an aggressive mostly T-cell lymphoma which occurs mainly in children and adolescents, where it accounts for about half of childhood lymphomas. About two-thirds of the patients are males. A second peak is seen again in patients over 40 years of age. The distinction between lymphoblastic lymphoma and acute lymphoblastic leukemia is, in part, arbitrarily, based on the degree of marrow involvement. The chief biologic difference is that lymphoblastic leukemias are predominantly B-cell diseases, unlike the extra-medullary, mostly T-cell lymphoblastic lymphomas.

T-Cell Prolymphocytic Leukemia (“T-PLL”)

T-cell prolymphocytic leukemia is a rare aggressive post-thymic malignancy with distinctive clinical and morphological and cytogenetic features (See review Matutes E. e.al., 1991 Blood, 78: 3269-74). T-PLL is resistant to chemotherapy and has a poor median survival (7.5 months). Although some patients may initially present with indolent disease they eventually progress and the outcome is then similar. New therapeutic approaches are needed to improve the outcome of this fatal disease.

Adult T-Cell Leukemia/Lymphoma (“ATL”)

Adult T-cell leukemia (“ATL”) is one of the T cell malignant neoplasms associated with human T cell leukemia virus type-I (HTLV-I). It is an aggressive fatal malignancy of mature CD4+ lymphocytes (See review Hatta e.al., 2002, Leukemia, 16: 1069-85; Yamada Y. 1983, Blood, 61: 192-9). ATL is prevalent in Southern Japan and the Carribbean basin and occurs sporadically in Africa, Latin America, the Middle East, and the United States. ATL has a poor prognosis due to an intrinsic resistance of leukaemic cells to conventional chemotherapy.

ATL has been classified into four main subtypes. In the relatively smoldering and chronic forms, the median survival is 2 years or more. In the acute or lymphomatous forms, the media survival is 13 months. Hematopoeiteic stem cell transplantation and chemotherapy has been used for the treatment of ATL.

Anaplastic Large Cell Lymphoma (Ki-30/CD-30)

Anaplastic large cell lymphoma (“ALCL”) can be systemic in children or young adults or cutaneous (in/on the skin). Disease limited to the skin is quite slow growing (indolent) and remains localized to the skin with many examples of spontaneous remission—this so-called “classic” ALCL is most common in children and adolescents and has a high frequency of gene translocation t(2;5). Primary cutaneous ALCL tends to occur more in adults and lacks the translocation. Most cases are T-cell or cell type unknown (null). The systemic form of ALCL may involve lymph nodes and extranodal sites. Chemotherapy has been used to treat the systemic form of ALCL.

TABLE 1
T-cell Lymphoproliferative Disorders

| | |
|---|---|
| T-cell and NK-cell Neoplasms | Nodular lymophocyte predominant |
| | Hodgkin lymphoma |
| | Classical Hodgkin lymphoma |
| | Nodular sclerosis classical |
| | Hodgkin lymphoma |
| | Lymphocyte-rich classical |
| | Hodgkin lymphoma |
| | Mixed cellularity classical Hodgkin |
| | lymphoma |
| | Lymphocyte-depleted classical |
| | Hodgkin lymphoma |
| Precursor T-cell Neoplasms | Precursor T lymphoblastic leukemia |
| | lymphoma |
| | Blastic NK cell lymphoma |
| Mature T-cell & NK | T-cell prolymphocytic leukemia |
| cell Neoplasms | T-cell large granular lymphocytic |
| | leukemia |
| | Aggressive NK cell leukemia |
| | Adult T-cell leukemia/lymphoma |
| | Extranodal NK/Tcell lymphoma, |
| | nasal type |
| | Enteropathy-type T-cell lymphoma |
| | Hepatosplenic T-cell lymphoma |
| | Primary cutaneous anaplastic |
| | large cell lymphoma |
| | Peripheral T-cell lymphoma, |
| | unspecified |
| | Angioimmunoblastic T-cell lymphoma |
| | Anaplastic large cell lymphoma |

### 2.3 Cancer Therapy

Currently, cancer therapy may involve surgery, chemotherapy, hormonal therapy and/or radiation treatment to eradicate neoplastic cells in a patient (see, for example, Stockdale, 1998, “Principles of Cancer Patient Management”, in Scientific American: Medicine, vol. 3, Rubenstein and Federman, eds., Chapter 12, Section IV). Recently, cancer therapy has also employed biological therapy or immunotherapy. All of these approaches pose significant drawbacks for the patient. Surgery, for example, may be contraindicated due to the health of the patient or may be unacceptable to the patient. Additionally, surgery may not completely remove the neoplastic tissue. Radiation therapy is only effective when the neoplastic tissue exhibits a higher sensitivity to radiation than normal tissue, and radiation therapy can also often elicit serious side effects. Hormonal therapy is rarely given as a single agent and although it can be effective, is often used to prevent or delay recurrence of cancer after other treatments have removed the majority of the cancer cells. Biological therapies/immunotherapies are limited in number and may produce side effects such as rashes or swellings, flu-like symptoms, including fever, chills and fatigue, digestive tract problems or allergic reactions.

With respect to chemotherapy, there are a variety of chemotherapeutic agents available for the treatment of cancer. A significant majority of cancer chemotherapeutics act by inhibiting DNA synthesis, either directly or indirectly by inhibiting the biosynthesis of the deoxyribonucleotide triphosphate precursors, to prevent DNA replication and concomitant cell division (see, for example, Gilman et al., Goodman and Gilman's: The Pharmacological Basis of Therapeutics, Eighth Ed. (Pergamom Press, New York, 1990)). These agents, which include alkylating agents such as nitrosourea, anti-metabolites such as methotrexate and hydroxyurea, and other agents such as, e.g., etoposides, campathecins, bleomycin, doxorubicin, and daunorubicin, although not necessarily cell cycle specific, kill cells during the S phase of the cell cycle because of their effect on DNA replication. Other agents, specifically colchicine and the vinca alkaloids, such as vinblastine and vincristine, interfere with microtubule assembly resulting in mitotic arrest. Chemotherapy protocols generally involve the administration of a combination of chemotherapeutic agents to increase the efficacy of treatment.

Despite the availability of a variety of chemotherapeutic agents, chemotherapy has many drawbacks (see, for example, Stockdale, 1998, “Principles Of Cancer Patient Management” in Scientific American Medicine, vol. 3, Rubenstein and Federman, eds., ch. 12, sect. 10). Almost all chemotherapeutic agents are toxic, and chemotherapy causes significant and often dangerous side effects, including severe nausea, bone marrow depression, immunosuppression, etc. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even those agents that act by mechanisms different from the mechanisms of action of the drugs used in the specific treatment; this phenomenon is termed pleiotropic drug or multidrug resistance. Thus, because of drug resistance, many cancers prove refractory to standard chemotherapeutic treatment protocols.

There is a significant need for alternative cancer treatments, particularly for the treatment of cancer that has proved refractory to standard cancer treatments, such as surgery, radiation therapy, chemotherapy, and hormonal therapy. Further, it is uncommon for cancer to be treated by only one method. Thus, there is a need for the development of new therapeutic agents for the treatment of cancer and new, more effective therapy combinations for the treatment of cancer.

### 2.4 T-Cell Surface Antigens

T-cells play a major role in the immune response by interacting with target cells and antigen-presenting cells. These interactions are highly specific and depend on the recognition of an antigen on the surface of a target or antigen-presenting cell by one of the specific antigen receptors on the surface of T-cells. The receptor-antigen interaction of T-cells and other cells is also facilitated by various T-cell surface proteins, e.g., the antigen receptor complex CD3 and accessory adhesion molecules such as CD4, LFA-1, CD8, and CD2.

The characteristic cell surface markers on T-cells have been the target for cancer therapies. Antibodies to T-cell surface markers, including CD2, CD3, CD4, CD11a and CD25 have been examined for example as immunosuppressive agents (See Berlin et al., 1992 Transplantation 53:840; Latinne et al., 1996 Int. Immunol. 8:1113).

This invention relates to the use of CD2 antagonists, specifically MEDI-507 (a humanized monoclonal antibody that recognizes the CD2 T-cell marker) in the prevention, treatment, management, or amelioration of cancer, particularly a T-cell malignancy, or one or more symptoms thereof. The human CD2 (T11) molecule is a 50 KDa surface glycoprotein expressed on >95% of thymocytes and virtually all peripheral T lymphocytes. CD2 acts as an adhesion molecule through the interaction with its primary ligand CD58 (or LFA-3) on target cells. Monoclonal antibodies to CD2 are known in the art, and they predominantly map to two sites of CD2 termed T11-1 (region 2) and T11-2 (region 1) (See Denning et al., 1987 J. Immunology 139:2573; Peterson et al., 1987 Nature: 329:842).

Citation or discussion of a reference herein shall not be construed as an admission that such is prior art to the present invention.

### 3. SUMMARY OF THE INVENTION

The present invention encompasses the use of MEDI-507, an analog, derivative or an antigen-binding fragment thereof as a single agent therapy for the prevention, treatment, management, or amelioration of cancer, particularly a T-cell malignancy, or one or more symptoms thereof. In particular, the invention encompasses the use of MEDI-507, an analog, derivative or an antigen-binding fragment thereof in treating subjects partially or completely refractory to current standard or experimental cancer therapies, particularly therapies for T-cell malignancies. The present invention provides methods for preventing, treating, managing or ameliorating cancer, particularly a T-cell malignancy, or one or more symptoms thereof, said methods comprising administering to a subject in need thereof a prophylactically or therapeutically effective amount of MEDI-507, an analog, derivative or an antigen-binding fragment thereof. In particular, the invention provides methods for preventing, treating, managing, or ameliorating indolent or aggressive T-cell leukemias or T-cell lymphomas, with the proviso that the T-cell lymphoma is not a cutaneous T-cell lymphoma, said methods comprising administering to a subject in need thereof a prophylactically or therapeutically effective amount of MEDI-507, an analog, derivative or an antigen-binding fragment thereof. In a specific embodiment, acute lymphoblastic leukemia, adult T-cell leukemia or Hodgkin's lymphoma is prevented, treated, managed or ameliorated by administering to a subject in need thereof a prophylactically or therapeutically effective amount of MEDI-507, an analog, derivative or an antigen-binding fragment thereof. In a preferred embodiment, systemic, non-cutaneous T-cell malignancies are prevented, treated, managed or ameliorated by administering to a subject in need thereof a prophylactically or therapeutically effective amount of MEDI-507, an analog, derivative or an antigen-binding fragment thereof.

The present invention also provide methods of preventing, treating, managing, or ameliorating cancer, particularly a T-cell malignancy, or one or more symptoms thereof, said methods comprising administering to a subject in need thereof MEDI-507, an analog, derivative or an antigen-binding fragment thereof conjugated to a therapeutic agent or drug. Examples of therapeutic agents which may be conjugated to MEDI-507, an analog, derivative or an antigen-binding fragment thereof include, but are not limited to, cytokines, toxins, radioactive elements, and antimetabolites. In a specific embodiment, a prophylactically or therapeutically effective amount of MEDI-507, an analog, derivative or an antigen-binding fragment thereof conjugated to an antibody specific for a tumor-associated antigen is administered to a subject in need thereof to prevent, treat, manage or ameliorate cancer, particularly a T-cell malignancy, or one or more symptoms thereof. In another embodiment, a prophylactically or therapeutically effective amount of MEDI-507, an analog, derivative or an antigen-binding fragment thereof conjugated to an antibody or ligand specific for an immune cell surface antigen other than CD2 is administered to a subject in need thereof to prevent, treat, manage or ameliorate cancer, particularly a T-cell malignancy or one or more symptoms thereof. In certain embodiments, MEDI-507, an analog, derivative or an antigen-binding fragment thereof conjugated to a toxin (e.g., a cytotoxin or an immunotoxin) or a radioactive element is not administered to a subject in need thereof to prevent, treat, manage, or ameliorate cancer, particularly a T-cell malignancy, or one or more symptoms thereof.

In one embodiment, the use of MEDI-507, an analog, derivative or an antigen-binding fragment thereof enhances the efficacy of standard or experimental treatment regimens for cancer. In a preferred embodiment, the use of MEDI-507, an analog, derivative or an antigen-binding fragment thereof enhances the efficacy of standard or experimental treatment regimens for T-cell malignancies (e.g., chemotherapy, radioimmunotherapy, or radiotherapy). In another embodiment, the use of MEDI-507, an analog, derivative or an antigen-binding fragment thereof prolongs the survival of a subject diagnosed with a T-cell malignancy.

The invention encompasses the use of MEDI-507, an analog, derivative, or an antigen-binding fragment thereof in combination with a standard or experimental cancer therapy for the prevention, treatment or amelioration of cancer, particularly a T-cell malignancy, or one or more symptoms thereof. The invention provides methods for preventing, treating, managing, or ameliorating cancer, particularly a T-cell malignancy, or one or more symptoms thereof, said methods comprising administering to a subject in need thereof a prophylactically or therapeutically effective amount of MEDI-507, an analog, derivative, or an antigen-binding fragment thereof, and one or more prophylactic or therapeutic agents, preferably prophylactic or therapeutic agents other than CD2 antagonists, which are currently being used, or have been used or are known to be useful in the prevention, treatment, management, or amelioration of cancer, particularly a T-cell malignancy, or one or more symptoms thereof. The invention also provides methods for preventing, treating, managing, or ameliorating cancer, particularly a T-cell malignancy, or one or more symptoms thereof, said methods comprising administering to a subject in need thereof a prophylactically or therapeutically effective amount of MEDI-507, an analog, derivative, or an antigen-binding fragment thereof conjugated to a therapeutic agent or drug, and one or more prophylactic or therapeutic agents, preferably prophylactic or therapeutic agents, other than CD2 antagonists, which are currently being used or have been used or are known to be useful for in the prevention, treatment, management, or amelioration of cancer, particularly a T-cell malignancy, or one or more symptoms thereof. Examples of therapeutic agents that can be used in combination with MEDI-507, an analog, derivative, or an antigen-binding fragment thereof for the prevention, treatment, management, or amelioration of cancer, include but are not limited to, chemotherapeutic agents, therapeutic antibodies, and angiogenesis inhibitors. Examples of therapeutic agents that are particularly useful in combination with MEDI-507, an analog, derivative, or an antigen-binding fragment thereof, for the prevention, treatment, management, or amelioration of T-cell malignancies, include but are not limited to, Campath®, anti-Tac, purine analogs, pentostatin, cytotoxic agents, anti-retroviral agents, arsenic trioxide, interferon-alpha, and anti-cancer agents. Chemotherapeutic agents that can be used in combination with MEDI-507, an analog, derivative, or an antigen-binding fragment thereof include but are not limited to alkylating agents, antimetabolites, natural products, and hormones. The combination therapies of the invention enable lower dosages of MEDI-507, an analog, derivative or an antigen-binding fragment thereof and/or less frequent administration of MEDI-507, an analog, derivative or an antigen-binding fragment thereof to a subject with cancer, particularly a T-cell malignancy, to achieve a therapeutic or prophylactic effect.

The invention provides pharmaceutical compositions for use in accordance with the methods of the invention, said pharmaceutical compositions comprising MEDI-507, an analog, derivative or an antigen-binding fragment thereof, in an amount effective to prevent, treat, manage or ameliorate cancer, particularly a T-cell malignancy, or one or more symptoms thereof and a pharmaceutically acceptable carrier. In a specific embodiment, a pharmaceutical composition comprises nucleic acid molecules encoding MEDI-507, an analog, derivative or an antigen-binding fragment thereof in an amount effective to prevent, treat, management, or ameliorate cancer, particularly a T-cell malignancy, or one or more symptoms thereof and a pharmaceutically acceptable carrier.

The invention provides pharmaceutical compositions for use in accordance with the methods of the invention, said pharmaceutical compositions comprising MEDI-507, an analog, derivative or an antigen-binding fragment thereof conjugated to a therapeutic agent or drug, in an amount effective to prevent, treat, manage, or ameliorate cancer, particularly a T-cell malignancy, or one or more symptoms thereof, and a pharmaceutically acceptable carrier. In certain embodiments, such pharmaceutical compositions do not comprise MEDI-507, an analog, derivative or an antigen-binding fragment thereof conjugated to a toxin or a radioactive element. The invention also provides pharmaceutical compositions for use in accordance with the methods of the invention, said pharmaceutical compositions comprising MEDI-507, an analog, derivative or an antigen-binding fragment thereof, a prophylactic or therapeutic agent other than a CD2 antagonist, and a pharmaceutically acceptable carrier.

In each of the various embodiments, pharmaceutical compositions comprising MEDI-507, an analog, derivative or an antigen-binding fragment thereof can be administered by parenteral administration (e.g., intradermal, intramuscular, intraperitoneal, intravenous and subcutaneous administration), epidural administration, topical administration, and mucosal administration (e.g., intranasal), or oral administration. In a specific embodiment, compositions comprising MEDI-507, an analog, derivative or an antigen-binding fragment thereof are administered subcutaneously, intramuscularly or intravenously.

In an alternative embodiment, the invention encompasses the use of one or more CD2 antagonists other than MEDI-507 for treating, preventing, managing or ameliorating cancer, particularly a T-cell malignancy, or one or more symptoms thereof. The present invention provides methods for preventing, treating, managing or ameliorating cancer, particularly T-cell malignancies or one or more symptoms thereof, said methods comprising administering to a subject in need thereof, a prophylactically or therapeutically effective amount of one or more CD2 antagonists, other than MEDI-507. The invention also provides methods for preventing, treating, managing or ameliorating cancer, particularly a T-cell malignancy, or one or more symptoms thereof, said methods comprising administering to a subject in need thereof, a CD2 antagonist conjugated to a therapeutic agent or drug. In certain embodiments, the CD2 antagonists used in the methods and compositions of the invention are not conjugated to a toxin or a radioactive element.

In a specific embodiment, the invention provides methods of preventing, treating, managing, or ameliorating cancer, particularly a T-cell malignancy, or one or more symptoms thereof, said methods comprising administering to a subject in need thereof a prophylactically or therapeutically effective amount of an antibody that immunospecifically binds to a CD2 epitope comprising amino acid residue 18, 55 or 59 of human CD2 (FIG. 1), with the proviso that said antibody is not MEDI-507 or LO-CD2a/BTI-322. In another embodiment, the invention provides methods of preventing, treating, managing, or ameliorating cancer, particularly a T-cell malignancy, or one or more symptoms thereof, said methods comprising administering to a subject in need thereof a prophylactically or therapeutically effective amount of an antibody that immunospecifically binds to a CD2 epitope comprising amino acid residues 18 and 55 of human CD2 (FIG. 1). In another embodiment, the invention provides methods of preventing, treating, managing, or ameliorating cancer, particularly a T-cell malignancy, or one or more symptoms thereof, said methods comprising administering to a subject in need thereof a prophylactically or therapeutically effective amount of an antibody that immunospecifically binds to a CD2 epitope comprising amino acid residues 18 and 59 of human CD2 (FIG. 1). In another embodiment, the invention provides methods of preventing, treating, managing, or ameliorating cancer, particularly a T-cell malignancy, or one or more symptoms thereof, said methods comprising administering to a subject in need thereof a prophylactically or therapeutically effective amount of an antibody that immunospecifically binds to a CD2 epitope comprising amino acid residues 55 and 59 of human CD2 (FIG. 1), with the proviso that said antibody is not MEDI-507 or LO-CD2a/BTI-322. In another embodiment, the invention provides the invention provides methods of preventing, treating, managing, or ameliorating cancer, particularly a T-cell malignancy, or one or more symptoms thereof, said methods comprising administering to a subject in need thereof a prophylactically or therapeutically effective amount of an antibody that immunospecifically binds to human CD2 or chimpanzee CD2 but not baboon CD2, with the proviso that said antibody is not MEDI-507 or LO-CD2a/BTI-322.

The invention provides methods of preventing, treating, managing or ameliorating cancer, particularly T-cell malignancies or one or more symptoms thereof, said methods comprising administering to a subject in need thereof, a prophylactically or therapeutically amount of one or more CD2 antagonists other than MEDI-507 in combination with other cancer therapies. The invention further provides pharmaceutical compositions and kits comprising one or more CD2 antagonists other than MEDI-507 for use in the prevention, treatment, management, or amelioration of cancer, particularly a T-cell malignancy, or one or more symptoms thereof.

### 3.1 Definitions

As used herein, the terms “T-cell malignancies” and “T-cell malignancy” refer to any T-cell lymphoproliferative disorder, including thymic and post-thymic malignancies. T-cell malignancies include tumors of T-cell origin. T-cell malignancies refer to tumors of lymphoid progenitor cell, thymocyte, T-cell, NK-cell, or antigen-presenting cell origin. In certain embodiments, the terms “T-cell malignancies” and “T-cell malignancy” refer to malignancies involving other cell types expressing a CD2 polypeptide which may be targeted in accordance with the invention, such as, e.g., cells involved in T-cell development, thymic stromal cells and thymic epithelial cells. T-cell malignancies include, but are not limited to, acute lymphoblastic leukemias, lymphomas, thymomas, acute lymphoblastic leukemias, and Hodgkin's and non-Hodgkin's disease, with the proviso that the T-cell malignancies are not cutaneous T-cell malignancies, in particular cutaneous T-cell lymphomas. In a preferred embodiment, the T-cell malignancies are systemic, non-cutaneous T-cell maligancies.

As used herein, the terms “adjunctive” and “conjunction” are used interchangeably with “in combination” or “combinatorial.”

As used herein, the term “analog” in the context of proteinaceous agents (e.g., proteins, polypeptides, and antibodies) refers to a proteinaceous agent that possesses a similar or identical function as a second proteinaceous agent but does not necessarily comprise a similar or identical amino acid sequence of the second proteinaceous agent, or possess a similar or identical structure of the second proteinaceous agent. A proteinaceous agent that has a similar amino acid sequence refers to a second proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second proteinaceous agent. A proteinaceous agent with similar structure to a second proteinaceous agent refers to a proteinaceous agent that has a similar secondary, tertiary or quaternary structure to the second proteinaceous agent. The structure of a polypeptide can be determined by methods known to those skilled in the art, including but not limited to, peptide sequencing, X-ray crystallography, nuclear magnetic resonance, circular dichroism, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity number of identical overlapping positions/total number of positions x 100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul e.al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul e.al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM 120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “analog” in the context of a non-proteinaceous agent refers to a second organic or inorganic molecule which possess a similar or identical function as a first organic or inorganic molecule and is structurally similar to the first organic or inorganic molecule.

As used herein, the terms “antagonist” and “antagonists” refer to any protein, polypeptide, peptide, antibody, antibody fragment, large molecule, or small molecule (less than 10 kD) that blocks, inhibits, reduces or neutralizes a function, activity and/or expression of another molecule. In various embodiments, an antagonist reduces a function, activity and/or expression of another molecule by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS).

As used herein, the terms “antibody” and “antibodies” refer to monoclonal antibodies, synthetic multispecific antibodies, human antibodies, humanized antibodies, camelised antibodies, single domain antibodies, chimeric antibodies, single-chain Fvs (scFv), single chain antibodies (including bispecific single chain antibodies), Fab fragments, F(ab′) fragments, disulfide-linked Fvs (sdFv), and anti-idiotypic (anti-Id) antibodies (including, e.g., anti-Id antibodies to antibodies of the invention), intrabodies, and epitope-binding fragments of any of the above. In particular, antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass.

As used herein, the term “CD2 polypeptide” refers to a CD2 glycoprotein (a.k.a. T11 or LFA-2) or a fragment thereof. In a preferred embodiment, a CD2 polypeptide is the cell surface 50-55 kDa glycoprotein expressed by immune cells such as T-cells and natural killer (“NK”) cells. The CD2 polypeptide may be from any species. In certain embodiments, a CD2 polypeptide is a human or chimpanzee CD2 molecule. In other embodiments, a CD2 polypeptide is not a baboon CD2 molecule. The nucleotide and/or amino acid sequences of CD2 polypeptides can be found in the literature or public databases, or the nucleotide and/or amino acid sequences can be determined using cloning and sequencing techniques known to one of skill in the art. For example, the nucleotide sequence of human CD2 can be found in the GenBank database (see, e.g., Accession Nos. X06143, AH002740, and M16445). In a preferred embodiment, a CD2 polypeptide is a human CD2 molecule (see, e.g., FIG. 1).

As used herein, the term “compete” refers to agents that inhibit or reduce the binding of a CD2 binding molecule, in particular LO-CD2a/BTI-322 or MEDI-507, to a CD2 polypeptide as assessed by a competition ELISA assay or a BIACORE assay well-known to one skilled in the art or described herein (see, e.g., Section 5.8). In a specific embodiment, a therapeutic or prophylactic agent reduces the binding of a CD2 binding molecule to a CD2 polypeptide by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% relative to a control such as PBS as assessed by a competition ELISA assay or a BIAcore assay. In a preferred embodiment, an anti-CD2 antibody reduces the binding of LO-CD2a/BTI-322 or MEDI-507 to a CD2 polypeptide by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as PBS as assessed by a competition ELISA assay or a BlAcore assay.

As used herein, the term “derivative” in the context of proteinaceous agents (e.g., proteins, polypeptides, and antibodies) refers to a proteinaceous agent that comprises an amino acid sequence which has been altered by the introduction of amino acid residue substitutions, deletions or additions. The term “derivative” as used herein also refers to a proteinaceous agent which has been modified, i.e, by the covalent attachment of any type of molecule to the proteinaceous agent. For example, but not by way of limitation, an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative proteinaceous agent may be produced by chemical modifications using techniques known to those of skill in the art, including, but not limited to specific chemical cleavage, acetylation, formylation, metabolic synthesis of tunicamycin, etc. Further, a derivative proteinaceous agent may contain one or more non-classical amino acids. A proteinaceous agent derivative possesses a similar or identical function as the proteinaceous agent from which it was derived.

As used herein, the term “derivative” in the context of a non-proteinaceous agent refers to a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl or amine group. An organic molecule may also be esterified, alkylated and/or phosphorylated.

As used herein, the term “effective amount” refers to the amount of a therapy which is sufficient to reduce or ameliorate the severity and/or duration of cancer, (particularly a T-cell malignancy) or one or more symptoms thereof, prevent the advancement of cancer (particularly a T-cell malignancy) or one or more symptoms thereof, cause regression of cancer (particularly a T-cell malignancy) or one or more symptoms thereof, or enhance or improve the prophylactic or the therapeutic effect(s) of another therapy (e.g., a prophylactic of therapeutic agent).

As used herein, the term “epitopes” refers to fragments of a polypeptide or protein having antigenic or immunogenic activity in an animal, preferably in a mammal, and most preferably in a human. In particular, the term “CD2 epitope” as used herein refers to a fragment of a CD2 polypeptide having antigenic or immunogenic activity in an animal, preferably in a mammal, and most preferably in a human. An epitope having immunogenic activity is a fragment of a polypeptide or protein that elicits an antibody response in an animal. An epitope having antigenic activity is a fragment of a polypeptide or protein to which an antibody immunospecifically binds as determined by any method well-known to one of skill in the art, for example by immunoassays. Antigenic epitopes need not necessarily be immunogenic.

As used herein, the term “fragment” refers to a peptide or polypeptide (including, but not limited to an antibody) comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of another polypeptide. In a specific embodiment, a fragment of a polypeptide retains at least one function of the polypeptide.

As used herein, the term “functional fragment” refers to a peptide or polypeptide (including, but not limited to an antibody) comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of second, different polypeptide, wherein said peptide or polypeptide retains at least one function of the second, different polypeptide.

As used herein, the term “fusion protein” refers to a polypeptide that comprises an amino acid sequence of a first protein or functional fragment, analog or derivative thereof, and an amino acid sequence of a heterologous protein (i.e., a second protein or functional fragment, analog or derivative thereof different than the first protein or functional fragment, analog or derivative thereof). In one embodiment, a fusion protein comprises a prophylactic or therapeutic agent fused (i.e., operably linked) to a heterologous protein, polypeptide or peptide. In accordance with this embodiment, the heterologous protein, polypeptide or peptide may or may not be a different type of prophylactic or therapeutic agent. In certain embodiments a fusion protein comprises a protein, polypeptide, or peptide with CD2 antagonist activity and a heterologous protein, polypeptide, or peptide. In other embodiments, a fusion protein comprises a CD2 binding molecule and a heterologous protein, polypeptide, or peptide. In a specific embodiment, a fusion protein comprises MEDI-507, an analog, derivative or an antigen-binding fragment thereof and a heterologous polypeptide. In accordance with these embodiments, a heterologous polypeptide is at least 5 amino acids residues, at least 10 amino acids residues, at least 15 amino acid residues, at least 20 amino acid residues, at least 25 amino acid residues, at least 30 acid residues, at least 40 amino acid residues, at least 50 amino acid residues, at least 75% amino acid residues, at least 100 amino acid residues, or at least 150 amino acid residues.

As used herein, the term “host cell” includes a particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the term “hybridizes under stringent conditions” describes conditions for hybridization and washing under which nucleotide sequences at least 60% (preferably, at least 65%, at least 70%, at least 75%, at least 80%, at least 85%, at least 90%, or at least 95%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non-limiting example stringent hybridization conditions are hybridization at 6×sodium chloride/sodium citrate (SSC) at about 45° C., followed by one or more washes in 0.1×SSC, 0.2% SDS at about 68° C. In a preferred, non-limiting example stringent hybridization conditions are hybridization in 6×SSC at about 45° C., followed by one or more washes in 0.2×SSC, 0.1% SDS at 50-65° C. (i.e., one or more washes at 50° C., 55 ° C., 60° C., or 65° C.). It is understood that the nucleic acids of the invention do not include nucleic acid molecules that hybridize under these conditions solely to a nucleotide sequence consisting of only A or T nucleotides.

As used herein, the term “immunospecifically binds to an antigen” and analogous terms refer to peptides, polypeptides, fusion proteins and antibodies or fragments thereof that specifically bind to an antigen or a fragment and do not specifically bind to other antigens. A peptide or polypeptide that immunospecifically binds to an antigen may bind to other peptides or polypeptides with lower affinity as determined by, e.g., immunoassays, BIAcore, or other assays known in the art. Antibodies or fragments that immunospecifically bind to an antigen may cross-reactive with related antigens. Preferably, antibodies or fragments that immunospecifically bind to an antigen do not cross-react with other antigens.

As used herein, the term “immunospecifically binds to a CD2 polypeptide” and analogous terms refer to peptides, polypeptides, fusion proteins and antibodies or fragments thereof that specifically bind to a CD2 polypeptide or a fragment thereof and do not specifically bind to other polypeptides. A peptide or polypeptide that immunospecifically binds to a CD2 polypeptide may bind to other peptides or polypeptides with lower affinity as determined by, e.g., immunoassays, BIAcore, or other assays known in the art. Antibodies or fragments that immunospecifically bind to a CD2 polypeptide may be cross-reactive with related antigens. Preferably, antibodies or fragments that immunospecifically bind to a CD2 polypeptide or fragment thereof do not cross-react with other antigens. Antibodies or fragments that immunospecifically bind to a CD2 polypeptide can be identified, for example, by immunoassays, BIAcore, or other techniques known to those of skill in the art. An antibody or fragment thereof binds specifically to a CD2 polypeptide when it binds to a CD2 polypeptide with higher affinity than to any cross-reactive antigen as determined using experimental techniques, such as radioimmunoassays (RIA) and enzyme-linked immunosorbent assays (ELISAs). See, e.g., Paul, ed., 1989, Fundamental Immunology Second Edition, Raven Press, New York at pages 332-336 for a discussion regarding antibody specificity.

As used herein, the term “in combination” refers to the use of more than one therapies (e.g., one or more prophylactic and/or therapeutic agents). The use of the term “in combination” does not restrict the order in which prophylactic and/or therapeutic agents are administered to a subject with cancer, particularly a T-cell malignancy. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent (e.g., a second prophylactic or therapeutic agent) to a subject with cancer, particularly a T-cell malignancy.

As used herein, the term “isolated” in the context of a proteinaceous agent (e.g., peptide, polypeptide, fusion protein or antibody) refers to a proteinaceous agent which is substantially free of cellular material or contaminating proteins from the cell or tissue source from which it is derived, or substantially free of chemical precursors or other chemicals when chemically synthesized. The language “substantially free of cellular material” includes preparations of a proteinaceous agent in which the proteinaceous agent is separated from cellular components of the cells from which it is isolated or recombinantly produced. Thus, a proteinaceous agent that is substantially free of cellular material includes preparations of a proteinaceous agent having less than about 30%, 20%, 10%, or 5% (by dry weight) of heterologous protein (also referred to herein as a “contaminating protein”). When the proteinaceous agent is recombinantly produced, it is also preferably substantially free of culture medium, i.e., culture medium represents less than about 20%, 10%, or 5% of the volume of the protein preparation. When the proteinaceous agent is produced by chemical synthesis, it is preferably substantially free of chemical precursors or other chemicals, i.e., it is separated from chemical precursors or other chemicals which are involved in the synthesis of the proteinaceous agent. Accordingly such preparations of a proteinaceous agent have less than about 30%, 20%, 10%, 5% (by dry weight) of chemical precursors or compounds other than the proteinaceous agent of interest. In a specific embodiment, a CD2 antagonist or a CD2 binding molecule is isolated. In a preferred embodiment, MEDI-507, an analog, derivative or an antigen-binding fragment thereof is isolated.

As used herein, the term “isolated” in the context of nucleic acid molecules refers to a nucleic acid molecule which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, a nucleic acid molecule encoding a CD2 antagonist is isolated. In a preferred embodiment, a nucleic acid molecule encoding MEDI-507, an analog, derivative or an antigen-binding fragment thereof is isolated.

As used herein, the terms “manage,” “managing,” and “management” refer to the beneficial effects that a subject derives from a therapy (e.g., a prophylactic or therapeutic agent), which does not result in a cure of cancer, particularly a T-cell malignancy. In certain embodiments, a subject is administered one or more therapies (e.g., one or more prophylactic or therapeutic agents) to “manage” cancer, particularly a T-cell malignancy, so as to prevent the progression or worsening of the cancer.

As used herein, the terms “non-responsive” and “refractory” describe patients treated with a currently available prophylactic or therapeutic agent for cancer, particularly a T-cell malignancy, or one or more symptoms thereof, which is not clinically adequate to relieve one or more symptoms associated with cancer, particularly a T-cell malignancy, or one or more symptoms thereof Typically, such patients suffer from severe, persistently active disease and require additional therapy to ameliorate the symptoms associated with cancer, particularly a T-cell malignancy, or one or more symptoms thereof.

As used herein, the terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

As used herein, the terms “prophylactic agent” and “prophylactic agents” refer to any agent(s) which can be used in the prevention of cancer, particularly T-cell malignancies. In certain embodiments, the term “prophylactic agent” refers to a CD2 antagonist (e.g., MEDI-507, an analog, derivative or an antigen-binding fragment thereof). In certain other embodiments, the term “prophylactic agent” does not refer to a CD2 antagonist (e.g., MEDI-507, an analog, derivative or an antigen-binding fragment thereof). Preferably, a prophylactic agent is an agent which is known to be useful to, or has been or is currently being used to the prevent or impede the development, onset or progression of cancer, particularly T-cell malignancies.

As used herein, the terms “prevent”, “preventing” and prevention refer the inhibition of the development or onset of cancer (particularly, a T-cell malignancy) or the prevention, recurrence, onset, or development of one or more symptoms of cancer, particularly a T-cell malignancy, in a subject resulting from the administration of therapy (e.g., a prophylactic or therapeutic agent) or a combination of therapies (e.g., a combination of prophylactic and/or therapeutic agents).

As used herein, the term “prophylactically effective amount” refers to that amount of the prophylactic agent sufficient to result in the prevention of the recurrence or onset of cancer (particularly a T-cell malignancy) or one or more symptoms thereof.

As used herein, a “prophylactic protocol” refers to a regimen for dosing and timing the administration of one or more prophylactic agents.

A used herein, a “protocol” includes dosing schedules and dosing regimens. The protocols herein are methods of use and include prophylactic and therapeutic protocols.

As used herein, the phrase “side effects” encompasses unwanted and adverse effects of a therapy (e.g., prophylactic and/or therapeutic agent). Adverse effects are always unwanted, but unwanted effects are not necessarily adverse. An adverse effect from a prophylactic or therapeutic agent might be harmful or uncomfortable or risky.

As used herein, the term “small molecules” and analogous terms include, but are not limited to, organic or inorganic compounds (i.e,. including heteroorganic and organometallic compounds) having a molecular weight less than 1,000 grams per mole. In a preferred embodiment, “small molecules” encompass organic or inorganic compounds having a molecular weight less than 750 grams per mole. In yet another specific embodiment, “small molecules” encompass organic or inorganic compounds having a molecular weight less than 500 grams per mole. Salts, esters, and other pharmaceutically acceptable forms of such compounds are also encompassed.

As used herein, the terms “subject” and “patient” are used interchangeably. As used herein, the terms “subject” and “subjects” refer to an animal, preferably a mammal including, but not limited to, a non-primate (e.g., a cow, pig, horse, cat, dog, rat, and mouse) and a non-primate (e.g., a monkey such as a cynomolgous monkey and a human), and more preferably a human. In a specific embodiment, the subject is a human with cancer. In a preferred embodiment, the subject is a human with a T-cell malignancy other than a cutaneous T-cell lymphoma. In another embodiment, the subject is a non-human animal such as a bird (e.g., a quail, chicken, or turkey), a farm animal (e.g., a cow, horse, pig, or sheep), a pet (e.g., a cat, dog, or guinea pig), or a laboratory animal (e.g., an animal model for a T-cell malignancy, such as a chimpanzee or a mouse with a T-cell malignancy).

As used herein, the term “synergistic” refers to a combination of therapies (e.g., combination of prophylactic and/or therapeutic agents) which is more effective than the additive effects of any two or more single therapies (e.g., two or more single prophylactic or therapeutic agents). A synergistic effect of a combination of therapies (e.g., prophylactic or therapeutic agents) permits the use of lower dosages of one or more of the therapies (e.g., one or more prophylactic and/or therapeutic agents) and/or less frequent administration of said therapies to a subject with cancer, particularly a T-cell malignancy. The ability to utilize lower dosages of therapies (e.g., prophylactic and/or therapeutic agents) and/or to administer said therapies less frequently reduces the toxicity associated with the administration of said therapies to a subject without reducing the efficacy of said therapies in the prevention, treatment, management, or amelioration of cancer, particularly a T-cell malignancy, or one or more symptoms thereof In addition, a synergistic effect can result in improved efficacy of therapies (e.g., prophylactic and/or therapeutic agents) in the prevention, treatment, management, or amelioration of cancer, particularly a T-cell malignancy, or one or more symptoms thereof. Finally, synergistic effect of a combination of therapies (e.g., prophylactic and/or therapeutic agents) may avoid or reduce adverse or unwanted side effects associated with the use of any single therapy.

As used herein, the terms “therapy” and “therapies” can refer to any protocol(s), method(s), and/or agent(s) that can be used in the prevention, treatment, management, or amelioration of a cancer, particularly a T-cell malignancy, or one or more symptoms thereof. In certain embodiments, the terms “therapy” and “therapies” refer to an anti-cancer agent, biological therapy, supportive therapy, and/or other therapies useful in treatment, management, prevention, or amelioration of cancer, particularly a T-cell malignancy, or one or more symptoms thereof, known to one of skill in the art, for example, a medical professional, such as a physician.

As used herein, the terms “therapeutic agent” and “therapeutic agents” refer to any agent(s) which can be used in the prevention, treatment, management or amelioration of cancer, particularly a T-cell malignancy, or one or more symptoms thereof. In certain embodiments, the term “therapeutic agent” refers to a CD2 antagonist (e.g., MEDI-507, an analog, derivative or an antigen-binding fragment thereof). In certain other embodiments, the term “therapeutic agent” does not refer to a CD2 antagonist (e.g., MEDI-507, an analog, derivative or an antigen-binding fragment thereof). Preferably, a therapeutic agent is an agent which is known to be useful for, or has been or is currently being used for the prevention, treatment, management, or amelioration of cancer, particularly a T-cell malignancies, or one or more symptoms thereof.

As used herein, the term “therapeutically effective amount” refers to that amount of a therapy (e.g., a prophylactic or therapeutic agent) which is sufficient to reduce the severity of cancer (particularly, a T-cell malignancy), reduce the duration of cancer (particularly, a T-cell malignancy), ameliorate one or more symptoms of cancer (particularly, a T-cell malignancy), prevent or slow the advancement of cancer (particularly, a T-cell malignancy), cause regression of cancer (particularly, a T-cell malignancy), or enhance or improve the therapeutic effect(s) of another therapy (e.g., a prophylactic or therapeutic agent).

As used herein, the term “therapeutic protocol” refers to a regimen for dosing and timing the administration of one or more therapeutic agents.

As used herein, the terms “treat”, “treatment” and “treating” refer to the reduction or amelioration of the progression, severity, and/or duration of cancer, particularly a T-cell malignancy, or one or more symptoms thereof that results from the administration of one or more therapies (e.g., one or more prophylactic and/or therapeutic agents).

### 4. BRIEF DESCRIPTION OF THE FIGURES

FIG. 1. The human CD2 amino acid sequence (SEQ ID NO: 7) is depicted.

FIG. 2. Analysis of the binding of MEDI-507 to MET-1 adult T-cell leukemia (“ATL”) cells using fluorescence-activated cell sorter (“FACS”).

FIG. 3. Mean concentration of human beta-2 microglobulin (“β₂μ”) in nonobese diabetic (“NOD”)/severe combined immunodeficient (“SCID”) mice injected with MET-1 leukemic cells and administered 4 weekly doses of PBS, 4 weekly doses of 100 μg MEDI-507, 4 weekly doses of 100 μg HAT, 4 weekly doses of 100 μg MEDI-507 with humanized anti-Tac (“HAT”), and weekly doses of 100 μg of MEDI-507 for 6 months.

FIG. 4. Kaplan-Meier survival plot of NOD/SCID mice injected with MET-1 leukemic cells and administered 4 weekly doses of PBS, 4 weekly doses of 100 μg MEDI-507, 4 weekly doses of 100 μg HAT, 4 weekly doses of 100 μg MEDI-507 with HAT, weekly doses of 100 μg MEDI-507 for six months, and NOD/SCID mice not injected with MET-1 leukemic cells and not administered a therapeutic agent.

FIG. 5. Changes in human β₂μ levels observed in NOD/SCID mice injected with MET-1 leukemic cells and administered weekly doses of 100 μg of MEDI-507 for six months.

FIG. 6. Kaplan-Meier survival plots of MET-1 FcRγ knock-out and FcRγ intact ATL-bearing NOD/SCID mice.

### 5. DETAILED DESCRIPTION OF THE INVENTION

The present invention encompasses treatment protocols that provide better prophylactic and therapeutic profiles than current single agent therapies or combination therapies for cancer, particularly a T-cell malignancy, or one or more symptoms thereof. The invention provides CD2 antagonist-based therapies for the prevention, treatment, management, or amelioration of cancer, particularly a T-cell malignancy, or one or more symptoms thereof. In particular, the invention provides prophylactic and therapeutic protocols for the prevention, treatment, management, or amelioration of cancer, particularly a T-cell malignancy, or one or more symptoms thereof, comprising the administration of MEDI-507, an analog, derivative or an antigen-fragment thereof to a subject in need thereof.

The present invention also provides pharmaceutical compositions and kits comprising a CD2 antagonist for use in the prevention, treatment, management, or amelioration of cancer, particularly a T-cell malignancy, or one or more symptoms thereof. In particular, the present invention provides pharmaceutical compositions and kits comprising MEDI-507, an analog, derivative or an antigen-binding fragment thereof.

### 5.1 MEDI-507, Derivatives, Analogs, Antigen-Binding Fragments Thereof

The present invention encompasses the use of MEDI-507 (MedImmune, Inc., Gaithersburg, Md.; Branco et al., 1999, Transplantation 68(10):1588-1596), an analog, derivative or an antigen-binding fragment thereof (e.g., one or more complementarity determining regions (“CDRs”) of MEDI-507) in the prevention, treatment, management or amelioration of cancer, particularly a T-cell malignancy, or one or more symptoms thereof. MEDI-507 is disclosed, e.g., in International Publication No. WO 99/03502, International Application Nos. PCT/US02/22273 and PCT/US02/06761, and U.S. application Ser. Nos. 09/462,140, 10/091,268, and 10/091,313, each of which is incorporated herein by reference in its entirety. MEDI-507 is a humanized IgG1κ class monoclonal antibody that immunospecifically binds to human CD2 polypeptide. MEDI-507 was constructed using molecular techniques to insert the CDRs from the rat monoclonal antibody LO-CD2a/BTI-322 into a human IgG1 framework. LO-CD2a/BTI-322 has the amino acid sequence disclosed, e.g., in U.S. Pat. Nos. 5,730,979, 5,817,311, and 5,951,983; and U.S. application Ser. Nos. 09/056,072 and 09/462,140 (each of which is incorporated herein by reference in its entirety), or the amino acid sequence of the monoclonal antibody produced by the cell line deposited with the American Type Culture Collection (ATCC(®), 10801 University Boulevard, Manassas, Va. 20110-2209 on Jul. 28, 1993 as Accession Number HB 11423.

The present invention encompasses the use of antibodies that immunospecifically bind to a CD2 polypeptide in the prevention, treatment, management or amelioration of cancer, particularly a T-cell malignancy, or one or more symptoms thereof, said antibodies comprising a variable heavy (“VH”) domain having an amino acid sequence of the VH domain for LO-CD2a/BTI-322 or MEDI-507. In particular, the present invention encompasses single domain antibodies comprising two VH domains having the amino acid sequence of the VH domain of LO-CD2a/BTI-322 or MEDI-507. The present invention also encompasses the use of antibodies that immunospecifically bind to a CD2 polypeptide in the prevention, treatment, management, or amelioration of cancer, particularly a T-cell malignancy, or one or more symptoms thereof, said antibodies comprising a VH CDR having an amino acid sequence of any one of a VH CDR of LO-CD2a/BTI-322 or MEDI-507. In particular, the invention encompasses the use of antibodies that immunospecifically bind to a CD2 polypeptide in the prevention, treatment, management, or amelioration of cancer, particularly a T-cell malignancy, or one or more symptoms thereof, said antibodies comprising a VH CDR having an amino sequence of any one of the VH CDRs listed in Table 2.

TABLE 2
CDR Sequences Of LO-CD2a/BTI-322

| CDR | Sequence | SEQ ID NO: |
|---|---|---|
| VH1 | EYYMY | 1 |
| VH2 | RIDPEDGSIDYVEKFKK | 2 |
| VH3 | GKFNYRFAY | 3 |
| VL1 | RSSQSLLHSSGNTYLN | 4 |
| VL2 | LVSKLES | 5 |
| VL3 | MQFTHYPYT | 6 |

In one embodiment, antibodies that immunospecifically bind to a CD2 polypeptide and comprises a VH CDR1 having the amino acid sequence of SEQ ID NO:1 are used in the prevention, treatment, management or amelioration of cancer, particularly a T-cell malignancy, or one or more symptoms thereof. In another embodiment, antibodies that immunospecifically bind to a CD2 polypeptide and comprises a VH CDR2 having the amino acid sequence of SEQ ID NO: 2 are used in the prevention, treatment, management or amelioration of cancer, particularly a T-cell malignancy, or one or more symptoms thereof. In another embodiment, antibodies that immunospecifically bind to a CD2 polypeptide and comprises a VH CDR3 having the amino acid sequence of SEQ ID NO:3 are used in the prevention, treatment, management or amelioration of cancer, particularly a T-cell malignancy, or one or more symptoms thereof. In a preferred embodiment, antibodies that immunospecifically bind to a CD2 polypeptide and comprises a VH CDR1 having the amino acid sequence of SEQ ID NO:1, a VH CDR2 having the amino acid sequence of SEQ ID NO:2, and a VH CDR3 having the amino acid sequence of SEQ ID NO:3 are used in the prevention, treatment, management or amelioration of cancer, particularly a T-cell malignancy, or one or more symptoms thereof.

The present invention encompasses the use of antibodies that immunospecifically bind to a CD2 polypeptide in the prevention, treatment, management or amelioration of cancer, particularly a T-cell malignancy, or one or more symptoms thereof, said antibodies comprising a variable light (“VL”) domain having an amino acid sequence of the VL domain for LO-CD2a/BTI-322 or MEDI-507. The present invention also encompasses the use of antibodies that immunospecifically bind to a CD2 polypeptide in the prevention, treatment, management or amelioration of cancer, particularly a T-cell malignancy, or one or more symptoms thereof, said antibodies comprising a VL CDR having an amino acid sequence of a VL CDR of LO-CD2a/BTI-322 or MEDI-507. In particular, the invention encompasses the use of antibodies that immunospecifically bind to a CD2 polypeptide in the prevention, treatment, management, or amelioration of cancer, particularly a T-cell malignancy, or one or more symptoms thereof, said antibodies comprising a VL CDR having an amino acid sequence of any one of the VL CDRs listed in Table 2, supra.

In one embodiment, antibodies that immunospecifically bind to a CD2 polypeptide and comprises a VL CDR1 having the amino acid sequence of SEQ ID NO:4 are used in the prevention, treatment, management or amelioration of cancer, particularly a T-cell malignancy, or one or more symptoms thereof. In another embodiment, antibodies that immunospecifically bind to a CD2 polypeptide and comprises a VL CDR2 having the amino acid sequence of SEQ ID NO:5 are used in the prevention, treatment, management, or amelioration of cancer, particularly a T-cell malignancy, or one or more symptoms thereof. In another embodiment, antibodies that immunospecifically bind to a CD2 polypeptide and comprises a VL CDR3 having the amino acid sequence of SEQ ID NO:6 are used in the prevention, treatment, management, or amelioration of cancer, particularly a T-cell malignancy, or one or more symptoms thereof. In a preferred embodiment, antibodies that immunospecifically bind to a CD2 polypeptide and comprises a VL CDR1 having the amino acid sequence of SEQ ID NO:4, a VL CDR2 having the amino acid sequence of SEQ ID NO:5, and a VL CDR3 having the amino acid sequence of SEQ ID NO: 6 are used in the prevention, treatment, management or amelioration of cancer, particularly a T-cell malignancy, or one or more symptoms thereof.

The present invention encompasses the use of antibodies that immunospecifically bind to a CD2 polypeptide for the prevention, treatment, management, or amelioration of cancer, particularly a T-cell malignancy, or one or more symptoms thereof, said antibodies comprising a VH domain disclosed herein combined with a VL domain disclosed herein, or other VL domain. The present invention also encompasses the use of antibodies that immunospecifically bind to a CD2 polypeptide for the prevention, treatment, management, or amelioration of a cancer, particularly a T-cell malignancy, or one or more symptoms thereof, said antibodies comprising a VL domain disclosed herein combined with a VH domain disclosed herein or other VH domain.

In particular, the present invention encompasses the use of antibodies that immunospecifically bind to a CD2 polypeptide in the prevention, treatment, management, treatment, or amelioration of a cancer, particularly a T-cell malignancy, or one or more symptoms thereof, said antibodies comprising one or more VH CDRs and one or more VL CDRs of LO-CD2a/BTI-322 or MEDI-507. The present invention also encompasses the use of antibodies that immunospecifically bind to a CD2 polypeptide for the prevention, treatment, management, or amelioration of cancer, particularly a T-cell malignancy, or one or more symptoms thereof, said antibodies comprising one or more VH CDRs and one or more VL CDRs listed in Table 2. More specifically, the invention encompasses the use of an antibody that immunospecifically binds to a CD2 polypeptide in the prevention, treatment, management, or amelioration of cancer, particularly a T-cell malignancy, or one or more symptoms thereof, said antibody comprising a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; or any combination thereof of the VH CDRs and VL CDRs listed in Table 2, supra.

In one embodiment, an antibody that immunospecifically binds to a CD2 polypeptide and comprises a VH CDR1 having the amino acid sequence of SEQ ID NO: 1 and a VL CDR1 having the amino acid sequence of SEQ ID NO: 4 is used in the prevention, treatment, management or amelioration of cancer, particularly a T-cell malignancy, or one or more symptoms thereof. In another embodiment, an antibody that immunospecifically binds to a CD2 polypeptide and comprises a VH CDR1 having the amino acid sequence of SEQ ID NO: 1 and a VL CDR2 having the amino acid sequence of SEQ ID NO: 5 is used in the prevention, treatment, management or amelioration of cancer, particularly a T-cell malignancy, or one or more symptoms thereof. In another embodiment, an antibody that immunospecifically binds to a CD2 polypeptide and comprises a VH CDR1 having the amino acid sequence of SEQ ID NO: 1 and a VL CDR3 having the amino acid sequence of SEQ ID NO: 6 is used in the prevention, treatment, management or amelioration of cancer, particularly a T-cell malignancy, or one or more symptoms thereof.

In another embodiment, an antibody that immunospecifically binds to a CD2 polypeptide and comprises a VH CDR2 having the amino acid sequence of SEQ ID NO: 2 and a VL CDR1 having the amino acid sequence of SEQ ID NO: 4 is used in the prevention, treatment, management or amelioration of cancer, particularly a T-cell malignancy, or one or more symptoms thereof In another embodiment, an antibody that immunospecifically binds to a CD2 polypeptide and comprises a VH CDR2 having the amino acid sequence of SEQ ID NO: 2 and a VL CDR2 having the amino acid sequence of SEQ ID NO: 5 is used in the prevention, treatment, management or amelioration of cancer, particularly a T-cell malignancy, or one or more symptoms thereof. In another embodiment, an antibody that immunospecifically binds to a CD2 polypeptide and comprises a VH CDR2 having the amino acid sequence of SEQ ID NO: 2 and a VL CDR3 having the amino acid sequence of SEQ ID NO: 6 is used in the prevention, treatment, management or amelioration of cancer, particularly a T-cell malignancy, or one or more symptoms thereof.

In another embodiment, an antibody that immunospecifically binds to a CD2 polypeptide and comprises a VH CDR3 having the amino acid sequence of SEQ ID NO: 3 and a VL CDR1 having the amino acid sequence of SEQ ID NO: 4 is used in the prevention, treatment, management or amelioration of cancer, particularly a T-cell malignancy, or one or more symptoms thereof. In another embodiment, an antibody that immunospecifically binds to a CD2 polypeptide and comprises a VH CDR3 having the amino acid sequence of SEQ ID NO: 3 and a VL CDR2 having the amino acid sequence of SEQ ID NO: 5 is used in the prevention, treatment, management or amelioration of cancer, particularly a T-cell malignancy, or one or more symptoms thereof. In a preferred embodiment, an antibody that immunospecifically binds to a CD2 polypeptide and comprises a VH CDR3 having the amino acid sequence of SEQ ID NO: 3 and a VL CDR3 having the amino acid sequence of SEQ ID NO: 6 is used in the prevention, treatment, management or amelioration of cancer, particularly a T-cell malignancy, or one or more symptoms thereof.

The present invention encompasses the use of a nucleic acid molecule, generally isolated, encoding MEDI-507, an analog, derivative or an antigen-binding fragment thereof in the prevention, treatment, management or amelioration of cancer, particularly a T-cell malignancy, or one or more symptoms thereof. In one embodiment, an isolated nucleic acid molecule encoding an antibody that immunospecifically binds to a CD2 polypeptide is used in the prevention, treatment, management or amelioration of cancer, particularly a T-cell malignancy, or one or more symptoms thereof, said antibody comprising a VH domain having the amino acid sequence of the VH domain of LO-CD2a/BTI-322 or MEDI-507. In another embodiment, an isolated nucleic acid molecule encoding an antibody that immunospecifically binds to a CD2 polypeptide is used in the prevention, treatment, management or amelioration of cancer, particularly a T-cell malignancy, or one or more symptoms thereof, said antibody comprising a VH domain having the amino acid sequence of the VH domain of the monoclonal antibody produced by the cell line deposited with the ATCC® as Accession Number HB 11423. In another embodiment, an isolated nucleic acid molecule encoding an antibody that immunospecifically binds to a CD2 polypeptide is used in the prevention, treatment, management, or amelioration of cancer, particularly a T-cell malignancy, or one or more symptoms thereof, said antibody comprising a VH CDR of LO-CD2a/BTI-322, MEDI-507, or the monoclonal antibody produced by the cell line deposited with the ATCC® as Accession Number HB 11423. In another embodiment, an isolated nucleic acid molecule encoding an antibody that immunospecifically binds to a CD2 polypeptide is used in the prevention, treatment, management or amelioration of cancer, particularly a T-cell malignancy, or one or more symptoms thereof, said antibody comprising a VH CDR1 having the amino acid sequence of the VH CDR1 listed in Table 2, supra. In another embodiment, an isolated nucleic acid molecule encoding an antibody that immunospecifically binds to a CD2 polypeptide is used in the prevention, treatment, management or amelioration of cancer, particularly a T-cell malignancy, or one or more symptoms thereof, said antibody comprising a VH CDR2 having the amino acid sequence of the VH CDR2 listed in Table 2, supra. In yet another embodiment, an isolated nucleic acid molecule encoding an antibody that immunospecifically binds to a CD2 polypeptide is used in the prevention, treatment, management or amelioration of cancer, particularly a T-cell malignancy, or one or more symptoms thereof, said antibody comprising a VH CDR3 having the amino acid sequence of the VH CDR3 listed in Table 2, supra.

In one embodiment, an isolated nucleic acid molecule encoding an antibody that immunospecifically binds to a CD2 polypeptide is used in the prevention, treatment, management or amelioration of cancer, particularly a T-cell malignancy, or one or more symptoms thereof, said antibody comprising a VL domain having the amino acid sequence of the VL domain of LO-CD2a/BTI-322 or MEDI-507. In another embodiment, an isolated nucleic acid molecule encoding an antibody that immunospecifically binds to a CD2 polypeptide is used in the prevention, treatment, management or amelioration of cancer, particularly a T-cell malignancy, or one or more symptoms thereof, said antibody comprising a VL domain having the amino acid sequence of the VL domain of the monoclonal antibody produced by the cell line deposited with the ATCC® as Accession Number HB 11423. In another embodiment, an isolated nucleic acid molecule encoding for an antibody that immunospecifically binds to a CD2 polypeptide is used in the prevention, treatment, management, or amelioration of cancer, particularly a T-cell malignancy, said antibody comprising of a VL CDR of LO-CD2a/BTI-322, MEDI-507, or the monoclonal antibody produced by the cell line deposited with the ATCC® as Accession Number HB 11423. In another embodiment, an isolated nucleic acid molecule encoding an antibody that immunospecifically binds to a CD2 polypeptide is used in the prevention, treatment, management or amelioration of cancer, particularly a T-cell malignancy, or one or more symptoms thereof, said antibody comprising a VL CDR1 having the amino acid sequence of the VL CDR1 listed in Table 2, supra. In another embodiment, an isolated nucleic acid molecule encoding an antibody that immunospecifically binds to a CD2 polypeptide is used in the prevention, treatment, management or amelioration of cancer, particularly a T-cell malignancy, or one or more symptoms thereof, said antibody comprising a VL CDR2 having the amino acid sequence of the VL CDR2 listed in Table 2, supra. In yet another embodiment, an isolated nucleic acid molecule encoding an antibody that immunospecifically binds to a CD2 polypeptide is used in the prevention, treatment, management or amelioration of cancer, particularly a T-cell malignancy, or one or more symptoms thereof, said antibody comprising a VL CDR3 having the amino acid sequence of the VL CDR3 listed in Table 2, supra.

In another embodiment, an isolated nucleic acid molecule encoding an antibody that immunospecifically binds to a CD2 polypeptide is used in the prevention, treatment, management or amelioration of cancer, particularly a T-cell malignancy, or one or more symptoms thereof, said antibody comprising a VH domain having the amino acid sequence of the VH domain of LO-CD2a/BTI-322 or MEDI-507 and a VL domain having the amino acid sequence of the VL domain of LO-CD2a/BTI-322 or MEDI-507. In another embodiment, an isolated nucleic acid molecule encoding an antibody that immunospecifically binds to a CD2 polypeptide is used in the prevention, management or amelioration of cancer, particularly a T-cell malignancy, or one or more symptoms thereof, said antibody comprising a VH domain having the amino acid sequence of the VH domain of the monoclonal antibody produced by the cell line deposited with the ATCC® as Accession Number HB 11423 and a VL domain having the amino acid sequence of the VL domain of the monoclonal antibody produced by the cell line deposited with the ATCC® as Accession Number HB 11423. In another embodiment, an isolated nucleic acid encoding an antibody that immunospecifically binds to a CD2 polypeptide is used in the prevention, treatment, management, or amelioration of cancer, particularly a T-cell malignancy, or one or more symptoms thereof, said antibody comprising a VH CDR of LO-CD2a/BTI-322, MEDI-507, or the monoclonal antibody produced by the cell line deposited with the ATCC® as Accession Number HB 11423 and a VL CDR of LO-CD2a/BTI-322, MEDI-507, or the monoclonal antibody produced by the cell line deposited with the ATCC® as Accession Number HB 11423. In another embodiment, an isolated nucleic acid molecule encoding an antibody that immunospecifically binds to a CD2 polypeptide is used in the prevention, treatment, management or amelioration of cancer, particularly a T-cell malignancy, or one or more symptoms thereof, said antibody comprising a VH CDR1, a VL CDR1, a VH CDR2, a VL CDR2, a VH CDR3, a VL CDR3, or any combination thereof having an amino acid sequence listed in Table 2, supra.

The present invention encompasses the use of antibodies that immunospecifically bind to a CD2 polypeptide in the prevention, treatment, management or amelioration of cancer, particularly a T-cell malignancy, or one or more symptoms thereof, said antibodies comprising derivatives of the VH domains, VH CDRs, VL domains, or VL CDRs described herein that immunospecifically bind to a CD2 polypeptide. Standard techniques known to those of skill in the art can be used to introduce mutations in the nucleotide sequence encoding an antibody of the invention, including, for example, site-directed mutagenesis and PCR-mediated mutagenesis which results in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, the derivatives have conservative amino acid substitutions that are made at one or more predicted non-essential amino acid residues (i.e., amino acid residues which are not critical for the antibody to immunospecifically bind to a CD2 polypeptide). A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains ( e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded antibody can be expressed and the activity of the antibody can be determined.

The present invention encompasses the use of antibodies that immunospecifically bind to a CD2 polypeptide in the prevention, treatment, management or amelioration of cancer, particularly a T-cell malignancy, or one or more symptoms thereof, said antibodies comprising the amino acid sequence of LO-CD2a/BTI-322 or MEDI-507 with one or more amino acid residue substitutions in the variable light (VL) domain and/or variable heavy (VH) domain. The present invention also encompasses the use of antibodies that immunospecifically bind to a CD2 polypeptide in the prevention, treatment, management or amelioration of cancer, particularly a T-cell malignancy, or one or more symptoms thereof, said antibodies comprising the amino acid sequence of LO-CD2a/BTI-322 or MEDI-507 with one or more amino acid residue substitutions in one or more VL CDRs and/or one or more VH CDRs. The antibody generated by introducing substitutions in the VH domain, VH CDRs, VL domain and/or VL CDRs of LO-CD2a/BTI-322 or MEDI-507 can be tested in vitro and/or in vivo, for example, for its ability to bind to a CD2 polypeptide, or for its ability to inhibit T-cell activation, or for its ability to inhibit T-cell proliferation, or for its ability to induce T-cell lysis, or for its ability to prevent, treat, manage, or ameliorate cancer, particularly a T-cell malignancy, or one or more symptoms thereof.

In a specific embodiment, the invention provides methods of preventing, treating, managing or ameliorating cancer, particularly T-cell malignancy, or one or more symptoms thereof, said methods comprising administering to a subject in need thereof an antibody that immunospecifically binds to a CD2 polypeptide comprising a nucleotide sequence that hybridizes to the nucleotide sequence encoding the monoclonal antibody produced by the cell line deposited with the ATCC® as Accession Number HB 11423 under stringent conditions, e.g., hybridization to filter-bound DNA in 6×sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3).

In a specific embodiment, the invention provides methods of preventing, treating, managing or ameliorating cancer, particularly a T-cell malignancy, or one or more symptoms thereof, said methods comprising administering to a subject in need thereof an antibody that immunospecifically binds to a CD2 polypeptide comprising a nucleotide sequence that hybridizes to the nucleotide sequence encoding MEDI-507 under stringent conditions, e.g., hybridization to filter-bound DNA in 6×sodium chloride/sodium citrate (SSC) at about 45 C followed by one or more washes in 0.2×SSC/0.1% SDS at about 50-65 C, under highly stringent conditions, e.g., hybridization to filter-bound nucleic acid in 6×SSC at about 45 C followed by one or more washes in 0.1×SSC/0.2% SDS at about 68 C, or under other stringent hybridization conditions which are known to those of skill in the art (see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3).

In a specific embodiment, the invention provides methods of preventing, treating, managing or ameliorating cancer, particularly T-cell malignancy, or one or more symptoms thereof, said methods comprising administering to a subject in need thereof an antibody that immunospecifically binds to a CD2 polypeptide, said antibody comprising an amino acid sequence of a VH domain or an amino acid sequence a VL domain encoded by a nucleotide sequence that hybridizes to the nucleotide sequence encoding the VH or VL domains of LO-CD2a/BTI-322 or MEDI-507 under stringent conditions, e.g., hybridization to filter-bound DNA in 6×sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45 C followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3).

In another embodiment, the invention provides methods of preventing, treating, managing or ameliorating cancer, particularly a T-cell malignancy, or one or more symptoms thereof, said methods comprising administering to a subject in need thereof an antibody that immunospecifically binds to a CD2 polypeptide, said antibody comprising an amino acid sequence of a VH CDR or an amino acid sequence of a VL CDR encoded by a nucleotide sequence that hybridizes to the nucleotide sequence encoding LO-CD2a/BTI-322 or MEDI-507 under stringent conditions. In another embodiment, the invention provides methods of preventing, treating, managing or ameliorating cancer, particularly a T-cell malignancy, or one or more symptoms thereof, said methods comprising administering to a subject in need thereof an antibody that immunospecifically binds to a CD2 polypeptide, said antibody comprising an amino acid sequence of a VH CDR or an amino acid sequence of a VL CDR encoded by a nucleotide sequence that hybridizes to the nucleotide sequence encoding any one of the VH CDRs or VL CDRs listed in Table 2, suptra, under stringent conditions. In another embodiment, the invention provides methods of preventing, treating, managing or ameliorating cancer, particularly a T-cell malignancy, or one or more symptoms thereof, said methods comprising administering to a subject in need thereof an antibody that immunospecifically binds to a CD2 polypeptide, said antibody comprising an amino acid sequence of a VH CDR or an amino acid sequence of a VL CDR encoded by a nucleotide sequence that hybridizes to the nucleotide sequence encoding any one of VH CDRs or VL CDRs of the monoclonal antibody produced by the cell line deposited with the ATCC® as Accession Number HB 11423 under stringent conditions.

In another embodiment, the invention provides methods of preventing, treating, managing or ameliorating cancer, particularly a T-cell malignancy, or one or more symptoms thereof, said methods comprising administering to a subject in need thereof an antibody that immunospecifically binds to a CD2 polypeptide, said antibody comprising an amino acid sequence of a VH CDR and an amino acid sequence of a VL CDR encoded by nucleotide sequences that hybridizes to the nucleotide sequences encoding LO-CD2a/BTI-322 or MEDI-507 under stringent conditions. In another embodiment, the invention provides methods of preventing, treating, managing or ameliorating cancer, particularly a T-cell malignancy, or one or more symptoms thereof, said methods comprising administering to a subject in need thereof an antibody that immunospecifically binds to a CD2 polypeptide, said antibody comprising an amino acid sequence of a VH CDR and an amino acid sequence of a VL CDR encoded by nucleotide sequences that hybridizes to the nucleotide sequences encoding any one of the VH CDRs and VL CDRs listed in Table 2, supra, under stringent conditions. In another embodiment, the invention provides methods of preventing, treating, managing or ameliorating cancer, particularly a T-cell malignancy, or one or more symptoms thereof, said methods comprising administering to a subject in need thereof an antibody that immunospecifically binds to a CD2 polypeptide, said antibody comprising an amino acid sequence of a VH CDR and an amino acid sequence of a VL CDR encoded by nucleotide sequences that hybridizes to the nucleotide sequences encoding the monoclonal antibody produced by the cell line deposited with the ATCC® as Accession Number HB 11423 under stringent conditions.

In a specific embodiment, the invention provides methods of preventing, treating, managing or ameliorating cancer, particularly a T-cell malignancy, or one or more symptoms thereof, said methods comprising administering to a subject in need thereof an antibody that immunospecifically binds to a CD2 polypeptide, said antibody comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of the monoclonal antibody produced by the cell line deposited with the ATCC® as Accession Number HB 11423. In another embodiment, the invention provides methods of preventing, treating, managing or ameliorating cancer, particularly a T-cell malignancy, or one or more symptoms thereof, said methods comprising administering to a subject in need thereof an antibody that immunospecifically binds to a CD2 polypeptide, said antibody comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of MEDI-507. In another embodiment, the invention provides methods of preventing, treating, managing or ameliorating cancer, particularly a T-cell malignancy, or one or more symptoms thereof, said methods comprising administering to a subject in need thereof an antibody that immunospecifically binds to a CD2 polypeptide, said antibody comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of LO-CD2a/BTI-322.

In another embodiment, the invention provides methods of preventing, treating, managing or ameliorating cancer, particularly a T-cell malignancy, or one or more symptoms thereof, said methods comprising administering to a subject in need thereof an antibody that immunospecifically binds to a CD2 polypeptide, said antibody comprising an amino acid sequence of a VH domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VH domain of MEDI-507. In another embodiment, the invention provides methods of preventing, treating, managing or ameliorating cancer, particularly a T-cell malignancy, or one or more symptoms thereof, said methods comprising administering to a subject in need thereof an antibody that immunospecifically binds to a CD2 polypeptide, said antibody comprising an amino acid sequence of a VH domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VH domain of LO-CD2a[BTI-322. In another embodiment, the invention provides methods of preventing, treating, managing or ameliorating cancer, particularly a T-cell malignancy, or one or more symptoms thereof, said methods comprising administering to a subject in need thereof an antibody that immunospecifically binds to a CD2 polypeptide, said antibody comprising an amino acid sequence of a VH domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VH domain of the monoclonal antibody produced by the cell line deposited with the ATCCOR as Accession Number HB 11423.

In another embodiment, the invention provides methods of preventing, treating, managing or ameliorating cancer, particularly T-cell malignancy, or one or more symptoms thereof, said methods comprising administering to a subject in need thereof an antibody that immunospecifically binds to a CD2 polypeptide, said antibody comprising an amino acid sequence of one or more VH CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any one of the VH CDRs of LO-CD2a/BTI-322. In another embodiment, the invention provides methods of preventing, treating, managing or ameliorating cancer, particularly T-cell malignancy, or one or more symptoms thereof, said methods comprising administering to a subject in need thereof an antibody that immunospecifically binds to a CD2 polypeptide, said antibody comprising an amino acid sequence of one or more VH CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any one of the VH CDRs of MEDI-507. In another embodiment, the invention provides methods of preventing, treating, managing or ameliorating cancer, particularly T-cell malignancy, or one or more symptoms thereof, said methods comprising administering to a subject in need thereof an antibody that immunospecifically binds to a CD2 polypeptide, said antibody comprising an amino acid sequence of one or more VH CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VH CDRs listed in Table 2, supra. In another embodiment, the invention provides methods of preventing, treating, managing or ameliorating cancer, particularly a T-cell malignancy, or one or more symptoms thereof, said methods comprising administering to a subject in need thereof an antibody that immunospecifically binds to a CD2 polypeptide, said antibody comprising an amino acid sequence of one or more VH CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of one of the VH CDRs of the monoclonal antibody produced by the cell line deposited with the ATCC® as Accession Number HB 11423.

In another embodiment, the invention provides methods of preventing, treating, managing or ameliorating cancer, particularly a T-cell malignancy, or one or more symptoms thereof, said methods comprising administering to a subject in need thereof an antibody that immunospecifically binds to a CD2 polypeptide, said antibody comprising an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VL domain of MEDI-507. In another embodiment, the invention provides methods of preventing, treating, managing or ameliorating cancer, particularly a T-cell malignancy, or one or more symptoms thereof, said methods comprising administering to a subject in need thereof an antibody that immunospecifically binds to a CD2 polypeptide, said antibody comprising an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VL domain of LO-CD2a/BTI-322. In another embodiment, the invention provides methods of preventing, treating, managing or ameliorating cancer, particularly a T-cell malignancy, or one or more symptoms thereof, said methods comprising administering to a subject in need thereof an antibody that immunospecifically binds to a CD2 polypeptide, said antibody comprising an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VL domain of the monoclonal antibody produced by the cell line deposited with the ATCC® as Accession Number HB 11423.

In another embodiment, the invention provides methods of preventing, treating, managing or ameliorating cancer, particularly T-cell malignancy, or one or more symptoms thereof, said methods comprising administering to a subject in need thereof an antibody that immunospecifically binds to a CD2 polypeptide, said antibody comprising an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs of MEDI-507. In another embodiment, the invention provides methods of preventing, treating, managing or ameliorating cancer, particularly T-cell malignancy, or one or more symptoms thereof, said methods comprising administering to a subject in need thereof an antibody that immunospecifically binds to a CD2 polypeptide, said antibody comprising an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs of LO-CD2a/BTI-322. In another embodiment, the invention provides methods of preventing, treating, managing or ameliorating cancer, particularly a T-cell malignancy, or one or more symptoms thereof, said methods comprising administering to a subject in need thereof an antibody that immunospecifically binds to a CD2 polypeptide, said antibody comprising an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs listed in Table 2, supra. In another embodiment, the invention provides methods of preventing, treating, managing or ameliorating cancer, particularly a T-cell malignancy, or one or more symptoms thereof, said methods comprising administering to a subject in need thereof an antibody that immunospecifically binds to a CD2 polypeptide, said antibody comprising an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs of the monoclonal antibody produced by the cell line deposited with the ATCC® as Accession Number HB 11423.

The present invention encompasses the use of antibodies that compete with LO-CD2a/BTI-322 or an antigen-binding fragment thereof for binding to the CD2 polypeptide in the prevention, treatment, management or amelioration of cancer, particularly a T-cell malignancy, or one or more symptoms thereof. In a preferred embodiment, the present invention encompasses the use of antibodies that compete with MEDI-507 or an antigen-binding fragment thereof for binding to the CD2 polypeptide in the prevention, treatment, management or amelioration of cancer, particularly a T-cell malignancy, or one or more symptoms thereof.

The invention encompasses the use of derivatives of MEDI-507 or an antigen-binding fragment thereof that are modified, i.e, by the covalent attachment of any type of molecule to the antibody, in the methods and compositions of the invention. For example, but not by way of limitation, derivatives of MEDI-507 or an antigen-binding fragment thereof include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The present invention encompasses the use of antibodies which immunospecifically bind to a CD2 polypeptide in the methods and compositions of the invention, said antibodies comprising the amino acid sequence of MEDI-507 with mutations (e.g., one or more amino acid substitutions) in the framework regions. In certain embodiments, antibodies which immunospecifically bind to a CD2 polypeptide comprise the amino acid sequence of MEDI-507 with one or more amino acid residue substitutions in the framework regions of the VH and/or VL domains.

The present invention further encompasses the use of antibodies which immunospecifically bind to a CD2 polypeptide in the methods and compositions of the invention, said antibodies comprising the amino acid sequence of MEDI-507 with mutations (e.g., one or more amino acid residue substitutions) in the variable and framework regions.

### 5.2 CD2 Antagonists

In addition to the use of MEDI-507, an analog, derivative, or an antigen-binding fragment thereof in the methods and compositions of the invention, other CD2 antagonists may be used in accordance with the invention. CD2 antagonists include, but are not limited to, proteinaceous molecules (e.g., proteins, polypeptides (e.g., soluble CD2 polypeptides and soluble LFA-3 polypeptides), peptides, fusion proteins (e.g., soluble CD2 polypeptides conjugated to a therapeutic moiety and soluble LFA-3 polypeptides conjugated to a therapeutic moiety), antibodies (e.g., anti-CD2 antibodies), and antibody fragments), nucleic acid molecules (e.g., CD2 antisense nucleic acid molecules, triple helices or nucleic acid molecules encoding proteinaceous molecules), organic molecules, inorganic molecules, small organic molecules, drugs, and small inorganic molecules that block, inhibit, reduce or neutralize a function, an activity and/or the expression of a CD2 polypeptide, expressed by an immune cell, preferably a T-cell or NK-cell. Additional examples and characteristics of CD2 antagonists are disclosed in Section 4.1 of International Application Nos. PCT/US02/22273 and PCT/US02/06761, and U.S. patent application Ser. Nos. 10/091,268 and 10/091,313, filed Mar. 3, 2002, the contents of each of which are incorporated herein by reference in their entirety. In some embodiments, a CD2 antagonist used in accordance with the methods of the invention is not a small organic molecule, a drug or an antisense molecule. CD2 antagonists can be identified using techniques well-known in the art or described herein (e.g., Section 5.8).

In certain embodiments, CD2 antagonists reduce a function, activity, and/or expression of a CD2 polypeptide in a subject with a T-cell malignancy. In other embodiments, the CD2 antagonists directly bind to a CD2 polypeptide and directly or indirectly modulate an activity and/or function of T-lymphocytes. In particular embodiments, CD2 antagonists inhibit or reduce T-cell activation or proliferation in a subject with a T-cell malignancy as determined by standard in vivo and/or in vitro assays described herein or well-known to those skilled in the art. In a specific embodiment, CD2 antagonists mediate the depletion of lymphocytes, in particular peripheral blood T-cells, in a subject with a T-cell malignancy as determined by standard in vivo and/or in vitro assays described herein or well-known to those skilled in the art. In another embodiment, CD2 antagonists directly or indirectly modulate an activity and/or function of T-lymphocytes by utilizing antibody-dependent cytotoxicity (ADCC).

In certain embodiments, CD2 antagonists inhibit or reduce the interaction between a CD2 polypeptide and LFA-3 in an in vivo and/or in vitro assay described herein (e.g., a competition ELISA) or known to one of skill in the art. In other embodiments, CD2 antagonists do not inhibit or interfere with the interaction between a CD2 polypeptide and LFA-3. In a specific embodiment, a CD2 antagonist reduces the interaction between a CD2 polypeptide and LFA-3 by at least 40%, at least 50%, at least 60%, at least 70%, at least 80%, at least 85%, at least 90%, at least 95%, or at least 99% as assessed by a competition assay well-known in the art or described herein, (e.g., a competition ELISA). In another specific embodiment, a CD2 antagonist reduces the interaction between a CD2 polypeptide and LFA-3 by less than 30%, less than 25%, less than 20%, less than 15%, less than 10%, or less than 5% as assessed by an assay well-known in the art or described herein (e.g., a competition ELISA).

In certain embodiments, CD2 antagonists modulate cytokine expression and/or release as determined by standard in vivo or in vitro assays described herein or well-known to one of skill in the art. In a specific embodiment, a CD2 antagonist modulates the concentration of cytokines such as, e.g., interferon-γ (“IFN-γ”), interleukin-2 (“IL-2”), interleukin-4 (“IL-4”), interleukin-6 (“IL-6”), interleukin-9 (“IL-9”), interleukin-12 (“IL-12”), and interleukin-15 (“IL-15”) in the serum of a subject administered a CD2 antagonist. Serum concentrations of cytokines can be measured by any technique well-known to one of skill in the art such as immunoassays, including, e.g., ELISA.

In a preferred embodiment, proteins, polypeptides or peptides (including antibodies and fusion proteins) that are utilized as CD2 antagonists are derived from the same species as the recipient of the proteins, polypeptides or peptides so as to reduce the likelihood of an immune response to those proteins, polypeptides or peptides. In another preferred embodiment, when the subject is a human, the proteins, polypeptides, or peptides that are utilized as CD2 antagonists are human or humanized.

Nucleic acid molecules encoding proteins, polypeptides, or peptides that function as CD2 antagonists can be administered to a subject with cancer, particularly a T-cell malignancy, in accordance with the methods of the invention. Further, nucleic acid molecules encoding derivatives, analogs, fragments or variants of proteins, polypeptides, or peptides that function as CD2 antagonists can be administered to a subject with cancer, particularly a T-cell malignancy in accordance with the methods of the invention. Preferably, such derivatives, analogs, variants and fragments retain the CD2 antagonist activity of the full-length wild-type protein, polypeptide, or peptide.

### 5.2.1 CD2 Binding Molecules

The present invention encompasses the use of CD2 antagonists referred to as CD2 binding molecules in the prevention, treatment, management, or amelioration of cancer, particularly a T-cell malignancy, or one or more symptoms thereof. The term “CD2 binding molecule” and analogous terms, as used herein, refer to a bioactive molecule that immunospecifically binds to a CD2 polypeptide and directly or indirectly modulates an activity and/or function of lymphocytes, in particular, peripheral blood T-cells. In one embodiment, CD2 binding molecules directly or indirectly mediate the depletion of lymphocytes, in particular peripheral blood T-cells. In a specific embodiment, the CD2 binding molecule binds to a CD2 polypeptide and preferentially mediates depletion of memory T cells (i.e., CD45RO+T cells) and not naive T cells. CD2 binding molecules can be identified, for example, by immunoassays or other techniques well-known to those of skill in the art. CD2 binding molecules include, but are not limited to, peptides, polypeptides, fusion proteins, small molecules, mimetic agents, synthetic drugs, organic molecules, inorganic molecules, and antibodies. Additional examples and characteristics of CD2 antagonists are disclosed in Section 4.2 of International Application Nos. PCT/US02/22273 and PCT/US02/06761, and U.S. patent application Ser. Nos. 10/091,268 and 10/091,313, filed Mar. 3, 2002, the contents of each of which are incorporated herein by reference in their entirety.

In one embodiment, a CD2 binding molecule is an antibody or an antigen-binding fragment thereof that immunospecifically binds to a CD2 polypeptide. In certain embodiments, the CD2 binding molecule is not MEDI-507, an analog, derivative or an antigen-binding fragment thereof, or LO-CD2a/BTI-322. In a preferred embodiment, a CD2 binding molecule is an antibody or an antigen-binding fragment thereof that immunospecifically binds to a CD2 polypeptide expressed by an immune cell such as a T-cell or NK cell. In another embodiment, a CD2 binding molecule is a polypeptide, peptide, a mimetic agent, an inorganic molecule or an organic molecule that immunospecifically binds to a CD2 polypeptide. In another embodiment, a CD2 binding molecule is an LFA-3 peptide, polypeptide, derivative, or analog thereof that immunospecifically binds to a CD2 polypeptide. In another embodiment, a CD2 binding molecule is a fusion protein that immunospecifically binds to a CD2 polypeptide. In a preferred embodiment, a CD2 binding molecule is a fusion protein that immunospecifically binds to a CD2 polypeptide expressed by an immune cell such as a T-cell or NK cell. In certain embodiments, a CD2 binding molecule is not a small organic molecule or a drug.

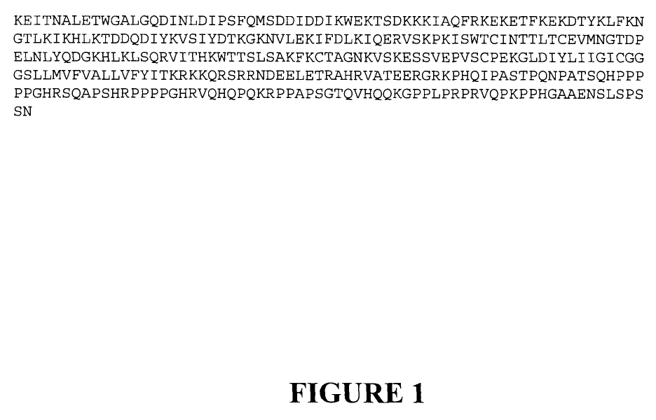

In a specific embodiment, the CD2 binding molecule immunospecifically binds to human and/or chimpanzee CD2 polypeptide but not to baboon CD2 polypeptide. In another embodiment, the CD2 binding molecule immunospecifically binds an epitope comprising amino acid residue 18, 55, and/or 59 of human CD2 (FIG. 1). In another embodiment, the CD2 binding molecule immunospecifically binds to an epitope comprising amino acid residues 18 and 55 of human CD2 (FIG. 1). In another embodiment, the CD2 binding molecule immunospecifically binds to an epitope comprising amino acid residues 18 and 59 of human CD2 (FIG. 1). In another embodiment, the CD2 binding molecule immunospecifically binds to an epitope comprising amino acid residues 55 and 59 of human CD2 (FIG. 1). In yet another embodiment, the CD2 binding molecule immunospecifically binds to an epitope comprising one or more of the 12 amino acid residues in the amino acid sequence of human CD2 or chimpanzee CD2 that are distinct from the amino acid residues found in the amino acid sequence of baboon CD2. In accordance with these embodiments, the CD2 binding molecule is preferably not LO-CD2a/BTI-322 or MEDI-507.

In certain embodiments, CD2 binding molecules inhibit or reduce the interaction between a CD2 polypeptide and LFA-3 in an in vivo and/or in vitro assay described herein (e.g., an ELISA) or known to one of skill in the art. In other embodiments, CD2 binding molecules do not inhibit or interfere with the interaction between a CD2 polypeptide and LFA-3.

5.2.1.1 Antibodies Other Than MEDI-507 that Immunospecifically Bind to CD2 Polypeptides

It should be recognized that antibodies that immunospecifically bind to a CD2 polypeptide are known in the art. Examples of known antibodies other than MEDI-507 described above that immunospecifically bind to a CD2 polypeptide include, but are not limited to, the murine monoclonal antibody produced by the cell line UMCD2 (Ancell Immunology Research Products, Bayport, Minn.; Kozarsky et al., 1993, Cell Immunol. 150:235-246), the murine monoclonal antibody produced by cell line RPA2.10 (Zymed Laboratories, Inc., San Francisco, Calif.; Rabinowitz et al., Clin. Immunol. & Immunopathol. 76(2):148-154), the rat monoclonal antibody LO-CD2b (International Publication No. WO 00/78814 A2), and the rat monoclonal antibody LO-CD2a/BTI-322 (Latinne et al., 1996, Int. Immunol. 8(7):1113-1119).

Antibodies that immunospecifically bind to a CD2 polypeptide include, but are not limited to, monoclonal antibodies, multispecific antibodies, human antibodies, humanized antibodies, camelised antibodies, single domain antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies that immunospecifically bind to a CD2 polypeptide include immunoglobulin molecules and immunologically active portions of immunoglobulin molecules, i.e., molecules that contain an antigen-binding site that immunospecifically bind to a CD2 polypeptide. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule. In a specific embodiment, the antibodies that immunospecifically bind to a CD2 polypeptide and mediate the depletion of T-cells comprise an Fe domain or a fragment thereof (e.g., the CH2, CH3, and/or hinge regions of an Fc domain). In a preferred embodiment, the antibodies that immunospecifically bind to a CD2 polypeptide and mediate the depletion of T cells comprise an Fe domain or fragment thereof that binds to an FcR, preferably an FcγRIII, expressed by an immune cell.

In certain embodiments, antibodies that immunospecifically bind to a CD2 polypeptide inhibit or reduce the interaction between a CD2 polypeptide and LFA-3 in an in vivo and/or in vitro assay described herein (e.g., an ELISA) or known to one of skill in the art. In other embodiments, antibodies that immunospecifically bind to a CD2 polypeptide do not inhibit or interfere with the interaction between a CD2 polypeptide and LFA-3.

In a specific embodiment, the antibody that immunospecifically binds to human and/or chimpanzee CD2 polypeptide but not to baboon CD2 polypeptide. In another embodiment, the antibody immunospecifically binds an epitope comprising amino acid residue 18, 55, and/or 59 of human CD2 (FIG. 1). In another embodiment, the antibody immunospecifically binds an epitope comprising amino acid residues 18 and 55 (FIG. 1). In another embodiment, the antibody immunospecifically binds an epitope comprising amino acid residues 18 and 59 (FIG. 1). In another embodiment, the antibody immunospecifically binds an epitope comprising amino acid residues 55 and 59 (FIG. 1). In yet another embodiment, the antibody immunospecifically binds to an epitope comprising one or more of the 12 amino acid residues in the amino acid sequence of human CD2 or chimpanzee CD2 that are distinct from the amino acid residues found in the amino acid sequence of baboon CD2. In accordance with these embodiments, the antibody is preferably not LO-CD2a/BTI-322 or MEDI-507.

The antibodies that immunospecifically bind to a CD2 polypeptide may be from any animal origin including birds and mammals (e.g., human, murine, donkey, sheep, rabbit, goat, guinea pig, camel, horse, or chicken). Preferably, the antibodies of the invention are human or humanized monoclonal antibodies. Human antibodies that immunospecifically bind to a CD2 polypeptide include antibodies having the amino acid sequence of a human immunoglobulin and antibodies isolated from human immunoglobulin libraries or from mice that express antibodies from human genes.

The antibodies that immunospecifically bind to a CD2 polypeptide may be monospecific, bispecific, trispecific or of greater multispecificity. Multispecific antibodies may be specific for different epitopes of a CD2 polypeptide or may be specific for both a CD2 polypeptide as well as for a heterologous epitope, such as a heterologous polypeptide or solid support material. See, e.g., PCT publications WO 93/17715, WO 92/08802, WO 91/00360, and WO 92/05793; Tutt, et al., J. Immunol. 147:60-69(1991); U.S. Pat. Nos. 4,474,893, 4,714,681, 4,925,648, 5,573,920, and 5,601,819; and Kostelny et al., J. Immunol. 148:1547-1553 (1992).

The present invention encompasses the use of antibodies that have a high binding affinity for a CD2 polypeptide in prevention, treatment, management or amelioration of cancer, particularly a T-cell malignancy, or one or more symptoms thereof. In a specific embodiment, an antibody that immunospecifically binds to a CD2 polypeptide has an association rate constant or kₒₙ rate (antibody (Ab)+antigen (Ag)^{kon}→Ab-Ag) of at least 10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹. In a preferred embodiment, an antibody that immunospecifically binds to a CD2 polypeptide has a kₒₙ rate of at least 2×10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹.

In another embodiment, an antibody that immunospecifically binds to a CD2 polypeptide has a k_{off} rate (antibody (Ab)+antigen (Ag)^{Koff}→Ab-Ag) of less than 10⁻¹s⁻¹, less than 5×10⁻¹s⁻¹, less than 10⁻²s⁻¹, less than 5×10⁻²s⁻¹, less than 10⁻³s⁻¹, less than 5×10⁻³s⁻¹, less than 10⁻⁴s⁻¹, less than 5×10⁻⁴s⁻¹, less than 10⁻⁵s⁻¹, less than 5×10⁻⁵s⁻¹, less than 10⁻⁶s⁻¹, less than 5×10⁻⁶s⁻¹, less than 10⁻⁷s⁻¹, less than 5×10⁻⁷s⁻¹, less than 10⁻⁸s⁻¹, less than 5×10⁻⁸s⁻¹, less than 10⁻⁹s⁻¹, less than 5×10⁻⁹s⁻¹, or less than 10⁻¹⁰s⁻¹. In a preferred embodiment, an antibody that immunospecifically binds to a CD2 polypeptide has a k_{off} rate of less than 5×10⁻⁴s⁻¹, less than 10⁻⁵s⁻¹, less than 5×10⁻⁵s⁻¹, less than 10⁻⁶s⁻¹, less than 5×10⁻⁶s⁻¹, less than 10⁻⁷s⁻¹, less than 5×10⁻⁷s⁻¹, less than 10⁻⁸s⁻¹, less than 5×10⁻⁸s⁻¹, less than 10⁻⁹s⁻¹, less than 5×10⁻⁹s⁻¹, or less than 10⁻¹⁰s⁻¹.

In another embodiment, an antibody that immunospecifically binds to a CD2 polypeptide has an affinity constant or Kₐ (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹. In yet another embodiment, an antibody that immunospecifically binds to a CD2 polypeptide has a dissociation constant or K_{d} (k_{off}/kₒₙ) of less than 10⁻² M, less than 5×10⁻² M, less than 10⁻³ M, less than 5×10⁻³ M, less than 10⁻⁴ M, less than 5×10⁻⁴ M, less than 10⁻⁵ M, less than 5×10⁻⁵ M, less than 10⁻⁶ M, less than 5×10⁻⁶ M, less than 10⁻⁷ M, less than 5×10⁻⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³ M, less than 5×10⁻¹³ M, less than 10⁻¹⁴ M, less than 5×10⁻¹⁴ M, less than 10⁻¹⁵ M, or less than 5×10⁻¹⁵ M.

In a specific embodiment, an antibody that immunospecifically binds to a CD2 polypeptide is LO-CD2a/BTI-322 or an antigen-binding fragment thereof (e.g., one or more complementarity determining regions (CDRs) of LO-CD2a/BTI-322). LO-CD2a/BTI-322 has the amino acid sequence disclosed, e.g., in U.S. Pat. Nos. 5,730,979, 5,817,311, and 5,951,983; and U.S. application Ser. Nos. 09/056,072 and 09/462,140 (each of which is incorporated herein by reference in its entirety), or the amino acid sequence of the monoclonal antibody produced by the cell line deposited with the American Type Culture Collection (ATCC®), 10801 University Boulevard, Manassas, Va. 20110-2209 on Jul. 28, 1993 as Accession Number HB 11423. In an alternative embodiment, an antibody that immunospecifically binds to a CD2 polypeptide is not LO-CD2a/BTI-322 or an antigen-binding fragment of LO-CD2a/BTI-322.

In another specific embodiment, an antibody that immunospecifically binds to a CD2 polypeptide is LO-CD2b or an antigen-binding fragment thereof (e.g., one or more CDRs of LO-CD2b). LO-CD2b has the amino acid sequence of the antibody produced by the cell line deposited with the ATCC®), 10801 University Boulevard, Manassas, Va. 20110-2209 on Jun. 22, 1999 as Accession Number PTA-802, or disclosed in, e.g., Dehoux et al., 2000, Transplantation 69(12):2622-2633 and International Publication No. WO 00/78814 (each of which is incorporated herein by reference in its entirety). In an alternative embodiment, an antibody that immunospecifically binds to a CD2 polypeptide is not LO-CD2b or an antigen-binding fragment of LO-CD2b.

### 5.2.1.2 LFA-3 Polypeptides that Immunospecifically Bind to CD2 Polypeptides

The present invention encompasses the use of LFA-3 peptides, polypeptides, derivatives and analogs thereof that immunospecifically bind to a CD2 polypeptide as CD2 antagonists in the prevention, treatment, management or amelioration of cancer, particularly a T-cell malignancy, or one or more symptoms thereof. Preferably, soluble LFA-3 polypeptides that immunospecifically bind to a CD2 polypeptide comprise at least 5, preferably at least 10, at least 20, at least 30, at least 40, at least 50, at least 60, at least 70, at least 80, at least 90 or at least 100 contiguous amino acid residues of LFA-3 are used to prevent, treat, manage or ameliorate cancer, particularly a T-cell malignancy, or one or more symptoms thereof. Soluble LFA-3 peptides, polypeptides, derivatives, and analogs thereof that immunospecifically bind to a CD2 polypeptide can be derived from any species. The nucleotide and/or amino acid sequences of LFA-3 can be found in the literature or public databases, or the nucleic acid and/or amino acid sequences can be determined using cloning and sequencing techniques well-known to one of skill in the art. For example, the nucleotide and amino acid sequences of human LFA-3 can be found in the GenBank databases (see, e.g., Accession Nos. E12817 and CAA29622).

In a specific embodiment, a soluble LFA-3 polypeptide that immunospecifically binds to a CD2 polypeptide consists the extracellular domain of naturally occurring LFA-3 or amino acid residues 1 to 187 of SEQ ID NO: 7 in International Application Nos. PCT/US02/22273 and PCT/US02/06761, and U.S. patent application Ser. Nos. 10/091,268 and 10/091,313. In another embodiment, a soluble LFA-3 polypeptide that immunospecifically binds to a CD2 polypeptide comprises a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 of SEQ ID NO: 7 in International Application Nos. PCT/US02/22273 and PCT/US02/06761, and U.S. patent application Ser. Nos. 10/091,268 and 10/091,313).

### 5.2.1.3 Fusion Proteins that Immunospecifically Bind to CD2 Polypeptides

The present invention encompasses the use of fusion proteins that immunospecifically bind to a CD2 polypeptide as CD2 antagonists in the prevention, treatment, management or amelioration of cancer, particularly a T-cell malignancy, or one or more symptoms thereof. In one embodiment, a fusion protein that immunospecifically binds to a CD2 polypeptide comprises a bioactive molecule fused to the Fc domain of an immunoglobulin molecule or a fragment thereof. In another embodiment, a fusion protein that immunospecifically binds to a CD2 polypeptide comprises a bioactive molecule fused to the CH2 and/or CH3 region of the Fc domain of an immunoglobulin molecule. In yet another embodiment, a fusion protein that immunospecifically binds to a CD2 polypeptide comprises a bioactive molecule fused to the CH2, CH3, and hinge regions of the Fc domain of an immunoglobulin molecule. In accordance with these embodiments, the bioactive molecule immunospecifically binds to a CD2 polypeptide. Bioactive molecules that immunospecifically bind to a CD2 polypeptide include, but are not limited to, peptides, polypeptides, small molecules, mimetic agents, synthetic drugs, inorganic molecules, and organic molecules. Preferably, a bioactive molecule that immunospecifically binds to a CD2 polypeptide is a polypeptide comprising at least 5, preferably at least 10, at least 20, at least 30, at least 40, at least 50, at least 60, at least 70, at least 80, at least 90 or at least 100 contiguous amino acid residues, and is heterologous to the amino acid sequence of the Fc domain of an immunoglobulin molecule or a fragment thereof.

In a specific embodiment, a fusion protein that immunospecifically binds to a CD2 polypeptide comprises LFA-3 or a fragment thereof which immunospecifically binds to a CD2 polypeptide fused to the Fc domain of an immunoglobulin molecule or a fragment thereof. In another embodiment, a fusion protein that immunospecifically binds to a CD2 polypeptide comprises LFA-3 or a fragment thereof which immunospecifically binds to a CD2 polypeptide fused to the CH2 and/or CH3 region of the Fc domain of an immunoglobulin molecule. In another embodiment, a fusion protein that immunospecifically binds to a CD2 polypeptide comprises LFA-3 or a fragment thereof which immunospecifically binds to a CD2 polypeptide fused to the CH2, CH3, and hinge regions of the Fc domain of an immunoglobulin molecule.

In another embodiment, a fusion protein that immunospecifically binds to a CD2 polypeptide comprises an extracellular domain of LFA-3 (e.g., amino acid residues 1 to 187 of SEQ ID NO:7 in International Application Nos. PCT/US02/22273 and PCT/US02/06761, and U.S. patent application Ser. Nos. 10/091,268 and 10/091,313) fused to the Fc domain of an immunoglobulin molecule or a fragment thereof. In another embodiment, a fusion protein that immunospecifically binds to a CD2 polypeptide comprises an extracellular domain of LFA-3 (e.g., amino acid residues 1 to 187 of SEQ ID NO:7 in International Application Nos. PCT/US02/22273 and PCT/US02/06761, and U.S. patent application Ser. Nos. 10/091,268 and 10/091,313) fused to the CH2 and/or CH3 region of the Fc domain of an immunoglobulin molecule. In another embodiment, a fusion protein that immunospecifically binds to a CD2 polypeptide comprises an extracellular domain of LFA-3 (e.g., amino acid residues 1 to 187 of SEQ ID NO:7 in International Application Nos. PCT/US02/22273 and PCT/US02/06761, and U.S. patent application Ser. Nos. 10/091,268 and 10/091,313) fused to the CH2, CH3, and hinge regions of the Fc domain of an immunoglobulin molecule.

In another embodiment, a fusion protein that immunospecifically binds to a CD2 polypeptide comprises a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 of SEQ ID NO:7 in International Application Nos. PCT/US02/22273 and PCT/US02/06761, and U.S. patent application Ser. Nos. 10/091,268 and 10/091,313) fused to the Fc domain of an immunoglobulin molecule or a fragment thereof. In another embodiment, a fusion protein that immunospecifically binds to a CD2 polypeptide comprises a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 of SEQ ID NO:7 in International Application Nos. PCT/US02/22273 and PCT/US02/06761, and U.S. patent application Ser. Nos. 10/091,268 and 10/091,313) fused to the CH2 and/or CH3 region of the Fc domain of an immunoglobulin molecule. In another embodiment, a fusion protein that immunospecifically binds to a CD2 polypeptide comprises a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 of SEQ ID NO:7 in International Application Nos. PCT/US02/22273 and PCT/US02/06761, and U.S. patent application Ser. Nos. 10/091,268 and 10/091,313) fused to the CH2, CH3, and hinge regions of the Fc domain of an immunoglobulin molecule.

In a specific embodiment, a CD2 binding molecule is LFA-3TIP (Biogen, Inc., Cambridge, Mass.). In an alterative embodiment, a CD2 binding molecule is not LFA-3TIP.

In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of LFA-3 or a fragment thereof fused to the Fc domain of an immunoglobulin molecule or a fragment thereof. In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of LFA-3 or a fragment thereof fused to the CH2 and/or CH3 region of the Fc domain of an immunoglobulin molecule. In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of LFA-3 or a fragment thereof fused to the CH2, CH3, and hinge regions of the Fc domain of an immunoglobulin molecule.

In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an extracellular domain of LFA-3 (e.g., amino acid residues 1 to 187 of SEQ ID NO:7 in International Application Nos. PCT/US02/22273 and PCT/US02/06761, and U.S. patent application Ser. Nos. 10/091,268 and 10/091,313) fused to the Fc domain of an immunoglobulin molecule or a fragment thereof. In another embodiment, a fusion protein that immunospecifically binds to a CD2 polypeptide comprise a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an extracellular domain of LFA-3 (e.g., amino acid residues 1 to 187of SEQ ID NO:7 in International Application Nos. PCT/US02/22273 and PCT/US02/06761, and U.S. patent application Ser. Nos. 10/091,268 and 10/091,313) fused to the CH2 and/or CH3 region of the Fc domain of an immunoglobulin molecule. In another embodiment, a fusion protein that immunospecifically binds to a CD2 polypeptide comprise a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an extracellular domain of LFA-3 (e.g., amino acid residues 1 to 187 of SEQ ID NO:7 in International Application Nos. PCT/US02/22273 and PCT/US02/06761, and U.S. patent application Ser. Nos. 10/091,268 and 10/091,313) fused to the CH2, CH3, and hinge regions of the Fc domain of an immunoglobulin molecule.

In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues I to 65, or amino acid residues 1 to 60 of SEQ ID NO:7 in International Application Nos. PCT/US02/22273 and PCT/US02/06761, and U.S. patent application Ser. Nos. 10/091,268 and 10/091,313) fused to the Fc domain of an immunoglobulin molecule or a fragment thereof.

In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 of SEQ ID NO:7 in International Application Nos. PCT/US02/22273 and PCT/US02/06761, and U.S. patent application Ser. Nos. 10/091,268 and 10/091,313) fused to the CH2 and/or CH3 region of the Fc domain of an immunoglobulin molecule.

In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 of SEQ ID NO:7 in International Application Nos. PCT/US02/22273 and PCT/US02/06761, and U.S. patent application Ser. Nos. 10/091,268 and 10/091,313) fused to the CH2, CH3, and hinge regions of the Fc domain of an immunoglobulin molecule.

In another embodiment, a fusion protein that immunospecifically binds to a CD2 polypeptide comprises the Fc domain of an immunoglobulin molecule or a fragment thereof fused to a polypeptide encoded by a nucleic acid molecule that hybridizes to the nucleotide sequence encoding LFA-3 or a fragment thereof.

Further, antibodies can be conjugated to albmin in order to make the antibody or antibody fragment more stable in vivo or have a longer half life in vivo. The techniques are well-known in the art, see, e.g., International Publication Nos. WO 93/15199, WO 93/15200, and WO 01/77137; and European Patent No. EP 413,622, all of which are incorporated herein in their entireties by reference.

### 5.3 CD2 Antagonists with Increased Half-Lives

The present invention encompasses the use of proteinaceous CD2 antagonists (preferably, MEDI-507, an analog, derivative, or an antigen-binding fragment thereof) that have extended half-lives in vivo in the prevention, treatment, management or amelioration of cancer, particularly a T-cell malignancy, or one or more symptoms thereof. In particular, the present invention provides proteinaceous CD2 antagonists (preferably, MEDI-507, an analog, derivative, or an antigen-binding fragment thereof) that have a half-life in an animal, preferably a mammal and most preferably a human, of greater than 3 days, greater than 7 days, greater than 10 days, preferably greater than 15 days, greater than 25 days, greater than 30 days, greater than 35 days, greater than 40 days, greater than 45 days, greater than 2 months, greater than 3 months, greater than 4 months, or greater than 5 months.

To prolong the serum circulation of proteinaceous CD2 antagonists (e.g., peptides, polypeptides, proteins, monoclonal antibodies, single chain antibodies and Fab fragments) in vivo inert polymer molecules such as high molecular weight polyethyleneglycol (PEG) can be attached to the antibodies with or without a multifunctional linker either through site-specific conjugation of the PEG to the N- or C-terminus of the polypeptide or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can, e.g., be tested for binding activity as well as for in vivo efficacy using methods well-known to those of skill in the art, for example, by immunoassays described herein.

Antibodies (preferably, MEDI-507, an analog, derivative, or an antigen-binding fragment thereof) having an increased half-life in vivo can also be generated introducing one or more amino acid modifications (i.e., substitutions, insertions or deletions) into an IgG constant domain, or FcRn binding fragment thereof (preferably a Fc or hinge-Fc domain fragment). See, e.g., International Publication No. WO 98/23289; International Publication No. WO 97/34631; and U.S. Pat. No. 6,277,375, each of which is incorporated herein by reference in its entirety.

### 5.4 CD2 Antagonist Conjugates

The present invention provides CD2 antagonists (preferably, MEDI-507, an analog, derivative or an antigen-binding fragment thereof) conjugated to a therapeutic agent or drug moiety that modifies a given biological response for use in the prevention, treatment, management or amelioration of cancer, particularly a T-cell malignancy, or one or more symptoms thereof. In a specific embodiment, CD2 antagonists other than MEDI-507, an analog, derivative or an antigen-binding fragment thereof are not conjugated to a therapeutic agent or drug moiety. In an alternative embodiment, CD2 antagonists other than MEDI-507, an analog, derivative or an antigen-binding fragment thereof are conjugated to a therapeutic agent or a drug moiety.

In certain embodiments, a CD2 antagonist such as, e.g., an anti-CD2 antibody (preferably, MEDT-507, an analog, derivative, or an antigen-binding fragment thereof) conjugated to a therapeutic agent or a drug moiety is used to prevent, treat, manage, or ameliorate cancer, preferably a T-cell malignancy, or one or more symptoms thereof. In other embodiments, a CD2 antagonist such as, e.g., an anti-CD2 antibody (preferably, MEDI-507, an analog, derivative, or an antigen-binding fragment thereof) that is not conjugated to a therapeutic agent or a drug moiety is used to prevent, treat, manage, or ameliorate cancer, preferably a T-cell malignancy, or one or more symptoms thereof. In yet other embodiments, a CD2 antagonist such as, e.g., an anti-CD2 antibody (preferably, MEDI-507, an analog, derivative, or an antigen-binding fragment thereof) conjugated to a therapeutic agent or drug moiety other than a toxin (e.g., cytotoxin or immunotoxin), a cytotoxic agent or a radioactive element is used to prevent, treat, manage, or ameliorate cancer, preferably a T-cell malignancy, or one or more symptoms thereof.

Therapeutic moieties include, but are not limited to, antimetabolites (e.g., methotrexate, 6-mercaptopurine, 6-thioguanine, cytarabine, 5-fluorouracil decarbazine); alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP), and cisplatin); anthracyclines (e.g., daunorubicin (formerly daunomycin) and doxorubicin); antibiotics (e.g., dactinomycin (formerly actinomycin), bleomycin, mithramycin, and anthramycin (AMC)); Auristatin molecules (e.g., auristatin PHE, bryostatin 1, and solastatin 10; see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference); hormones (e.g., glucocorticoids, progestins, androgens, and estrogens), DNA-repair enzyme inhibitors (e.g., etoposide or topotecan), kinase inhibitors (e.g., compound ST1571, imatinib mesylate (Kantarjian et al., Clin Cancer Res. 8(7):2167-76 (2002)); cytotoxic agents (e.g., paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucorticoids, procaine, tetracaine, lidocaine, propranolol, and puromycin and analogs or homologs thereof and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459); farmesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305); topoisomerase inhibitors (e.g., camptothecin; irinotecan; SN-38; topotecan; 9-aminocamptothecin; GG-211 (GI 147211); DX-8951f; IST-622; rubitecan; pyrazoloacridine; XR-5000; saintopin; UCE6; UCE1022; TAN-1518A; TAN-1518B; KT6006; KT6528; ED-110; NB-506; ED-110; NB-506; and rebeccamycin); bulgarein; DNA minor groove binders such as Hoescht dye 33342 and Hoechst dye 33258; nitidine; fagaronine; epiberberine; coralyne; beta-lapachone; BC-4-1; bisphosphonates (e.g., alendronate, cimadronte, clodronate, tiludronate, etidronate, ibandronate, neridronate, olpandronate, risedronate, piridronate, pamidronate, zolendronate) HMG-CoA reductase inhibitors, (e.g., lovastatin, simvastatin, atorvastatin, pravastatin, fluvastatin, statin, cerivastatin, lescol, lupitor, rosuvastatin and atorvastatin); antisense oligonucleotides (e.g., those disclosed in the U.S. Pat. Nos. 6,277,832, 5,998,596, 5,885,834, 5,734,033, and 5,618,709); adenosine deaminase inhibitors (e.g., Fludarabine phosphate and 2-Chlorodeoxyadenosine); ibritumomab tiuxetan (Zevalin®); tositumomab (Bexxar®)) and pharmaceutically acceptable salts, solvates, clathrates, and prodrugs thereof.

Further, an antibody or fragment thereof may be conjugated to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567-1574), and VEGF (see, International Publication No. WO 99/23105), an anti-angiogenic agent, e.g., angiostatin, endostatin or a component of the coagulation pathway (e.g., tissue factor); or, a biological response modifier such as, for example, a lymphokine (e.g., interferon gamma (“IFN-γ”), interleukin-1 (“IL-1”), interleukin-2 (“IL-2”), interleukin-5 (“IL-5”), interleukin-6 (“IL-6”), interleuking-7 (“IL-7”), interleukin-10 (“IL-10”), interleukin-12 (“IL-12”), interleukin-15 (“IL-15”), interleukin-23 (“IL-23”), granulocyte macrophage colony stimulating factor (“GM-CSF”), and granulocyte colony stimulating factor (“G-CSF”)), or a growth factor (e.g., growth hormone (“GH”)), or a coagulation agent (e.g., calcium, vitamin K, tissue factors, such as but not limited to, Hageman factor (factor XII), high-molecular-weight kininogen (HMWK), prekallikrein (PK), coagulation proteins-factors II (prothrombin), factor V, XIIa, VIII, XIIIa, XI, XIa, IX, IXa, X, phospholipid. fibrinopeptides A and B from the α and β chains of fibrinogen, fibrin monomer). In a specific embodiment, an antibody that immunospecifically binds to an IL-9 polypeptide is conjugated with a leukotriene antagonist (e.g., montelukast, zafirlukast, pranlukast, and zyleuton).

Moreover, an antibody can be conjugated to therapeutic moieties such as a radioactive metal ion, such as alph-emiters such as ²¹³Bi or macrocyclic chelators useful for conjugating radiometal ions, including but not limited to, ¹³¹In, ¹³¹LU, ¹³¹Y, ¹³¹Ho, ¹³¹Sm, to polypeptides. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Alternatively, an antibody can be conjugated to a second antibody to form an antibody heteroconjugate as described by Segal in U.S. Pat. No. 4,676,980, which is incorporated herein by reference in its entirety.

The present invention also provides CD2 antagonists, preferably, MEDI-507, an analog, derivative or an antigen-binding fragment thereof, conjugated to a diagnostic agent. MEDI-507, an analog, derivative or an antigen-binding fragment thereof can be used diagnostically, for example, to monitor the development or progression of cancer, particularly a T-cell malignancy, of a clinical testing procedure to, e.g., determine the efficacy of a given treatment regimen. Detection can be facilitated by coupling CD2 antagonists, preferably MEDI-507, an analog, derivative or an antigen-binding fragment thereof to a detectable substance. Examples of detectable substances include various enzymes, prosthetic groups, fluorescent materials, luminescent materials, bioluminescent materials, radioactive materials, positron emitting metals, and non-radioactive paramagnetic metal ions. The detectable substance may be coupled or conjugated either directly to the antibody or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Such diagnosis and detection can be accomplished by coupling the antibody to detectable substances including, but not limited to, various enzymes, enzymes including, but not limited to, horseradish peroxidase, alkaline phosphatase, beta-galactosidase, or acetylcholinesterase; prosthetic group complexes such as, but not limited to, streptavidin/biotin and avidin/biotin; fluorescent materials such as, but not limited to, umbelliferone, fluorescein, fluorescein isothiocyanate, rhodamine, dichlorotriazinylamine fluorescein, dansyl chloride or phycoerythrin; luminescent material such as, but not limited to, luminol; bioluminescent materials such as, but not limited to, luciferase, luciferin, and aequorin; radioactive material such as, but not limited to, bismuth (²¹³Bi), carbon (¹⁴C), chromium (⁵¹ Cr), cobalt (⁵⁷Co), fluorine (¹⁸F), gadolinium (¹⁵³ Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanSthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵ Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, 175Y), yttrium (⁹⁰Y), zinc (⁶⁵Zn); positron emitting metals using various positron emission tomographies, and nonradioactive paramagnetic metal ions.

### 5.5 Agents that may be Used in Combination with CD2 Antagonists for the Prevention or Treatment of Cancer

The invention also provides compositions comprising a CD2 antagonist (preferably, MEDI-507, an analog, derivative, or antigen-binding fragment thereof) and one or more prophylactic or therapeutic agents other than CD2 antagonists and methods for preventing, treating or ameliorating cancer, particularly a T-cell malignancy, or one or more symptoms thereof comprising administering to a subject in need thereof said compositions. Therapeutic or prophylactic agents include, but are not limited to, small molecules, synthetic drugs, peptides, polypeptides, proteins, nucleic acids (e.g., DNA and RNA nucleotides including, but not limited to, antisense nucleotide sequences, triple helices and nucleotide sequences encoding biologically active proteins, polypeptides or peptides) antibodies, synthetic or natural inorganic molecules, mimetic agents, and synthetic or natural organic molecules. Any agent which is known to be useful, or which has been used or is currently being used for the prevention, treatment, management, or amelioration of cancer, particularly a T-cell malignancy, or one or more symptoms thereof can be used in combination with a CD2 antagonist in accordance with the invention described herein. See, e.g., Hardman et al., eds., 1996, Goodman & Gilman's The Pharmacological Basis Of Basis Of Therapeutics 9^{th} Ed, Mc-Graw-Hill, New York and the emedicine website for information regarding prophylactic or therapeutic agents which have been or are currently being used for treating cancer, in particular a T-cell malignancy, or one or more symptoms thereof.

### 5.5.1 Anti-Cancer Agents and Therapeutic Antibodies

Examples of anti-cancer agents that can be used in the various embodiments of the invention, including pharmaceutical compositions and dosage forms and kits of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarnycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-I receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphory lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ornaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermnin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin.

Examples of therapeutic antibodies that can be used in methods of the invention include but are not limited to HERCEPTIN® (Trastuzumab) (Genentech, Calif.) which is a humanized anti-HER2 monoclonal antibody for the treatment of patients with metastatic breast cancer; REOPRO® (abeiximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-1A cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); LYMPHOCIDE™ Y-90 (Immunomedics); Lymphoscan (Tc-99m-labeled; radioimaging; Immunomedics); Nuvion (against CD3; Protein Design Labs); CM3 is a humanized anti-ICAM3 antibody (ICOS Pharm); IDEC-114 is a primatied anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CD20-sreptdavidin (+biotin-yttrium 90; NeoRx); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β₂ antibody (Cambridge Ab Tech). In a specific embodiment, a CD2 antagonist is used in combination with VITAXIN™ for the prevention, treatment, management, or amelioration of cancer, in particular a T-cell malignancy, or one or more symptoms thereof.

Chemotherapeutic agents that can be used in the methods and compositions of the invention include but are not limited to alkylating agents, antimetabolites, natural products, or hormones. Examples of alkylating agents useful for the prevention, treatment, management, or amelioration of T-cell malignancies in the methods and compositions of the invention include but are not limited to, nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites useful for the prevention, treatment, management, or amelioration of T-cell malignancies in the methods and compositions of the invention include but are not limited to folic acid analog (e.g., methotrexate), or pyrimidine analogs (e.g., Cytarabine), purine analogs (e.g., mercaptopurine, thioguanine, pentostatin). Examples of natural products useful for the prevention, treatment, management, or amelioration of T-cell malignancies in the methods and compositions of the invention include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents useful for the treatment or prevention of cancer in the methods and compositions of the invention include but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites useful for the treatment or prevention of cancer in the methods and compositions of the invention include but are not limited to folic acid analog (e.g., methotrexate), or pyrimidine analogs (e.g., fluorouracil, floxouridine, Cytarabine), purine analogs (e.g., mercaptopurine, thioguanine, pentostatin). Examples of natural products useful for the treatment or prevention of cancer in the methods and compositions of the invention include but are not limited to vinca alkaloids (e.g., vinblastin, vincristine), epipodophyllotoxins (e.g., etoposide, teniposide), antibiotics (e.g., actinomycin D, daunorubicin, doxorubicin, bleomycin, plicamycin, mitomycin), enzymes (e.g., L-asparaginase), or biological response modifiers (e.g., interferon alpha). Examples of hormones and antagonists useful for the treatment or prevention of cancer in the methods and compositions of the invention include but are not limited to adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions of the invention for the treatment or prevention of cancer include platinume coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

### 5.5.2 Angiogenesis Inhibitors

The invention encompasses the use of one or more angiogenesis inhibitors in combination with a CD2 antagonist to prevent, treat, manage, or ameliorate cancer, particularly a T-cell malignancy, or one or more symptoms thereof. Examples of angiogenesis inhibitors include but not limited to: Angiostatin (plasminogen fragment); antiangiogenic antithrombin III; Angiozyme; ABT-627; Bay 12-9566; Benefin; Bevacizumab; BMS-275291; cartilage-derived inhibitor (CDI); CAI; CD59 complement fragment; CEP-7055; Col 3; Combretastatin A-4; Endostatin (collagen XVIII fragment); fibronectin fragment; Gro-beta; halofuginone; heparinases; hparin hexasaccharide fragment; HMV833; human chorionic gonadotropin (hCG); IM-862; interferon alpha/beta/gamma; interferon inducible protein (IP-10); interleukin-12; Kringle 5 (plasminogen fragment); marimastat; metalloproteinase inhibitors (TIMPs); 2-methoxyestradiol; MMI 270 (CGS 27023A); MoAb IMC-1C11; Neovastat; NM-3; Panzem; PI-88; placental ribonuclease inhibitor; plasminogen activator inhibitor; Platelet factor-4 (PF4); Prinomastat; prolactin 16 kD fragment; proliferin-related protein (PRP); PTK 787/ZK 222594; retinoids; Solimastat; Squalamine; SS 3304; SU 5416; SU6668; SU11248; tetrahydrocortisol-S; tetrathiomolybdate; thalidomide; thrombospondin-1 (TSP-1); TNP-470; transforming growth factor-beta (TGF-b); vasculostatin; vasostatin (calreticulin fragment); ZD6126; ZD 6474; farnesyl transferase inhibitors (FTI); and bisphosphonates.

### 5.6 Treatment Protocols

The present invention encompasses CD2-antagonists-based therapies which involve administering CD2 antagonists to an animal, preferably a mammal, and most preferably a human, for preventing, treating, managing, or ameliorating cancer, particularly a T-cell malignancy, or one or more symptoms thereof. In a preferred embodiment, the CD2 antagonist used in the therapeutic methods and compositions of the invention is MEDI-507, an analog, derivative, or an antigen-binding fragment thereof. In another preferred embodiment, the invention encompasses the use of MEDI-507, an analog, derivative or an antigen-binding fragment thereof as a single agent therapy for preventing, treating, managing, or ameliorating a T-cell malignancy or one or more symptoms associated with a T-cell malignancy.

The present invention also encompasses combination therapies that provide better prophylactic and therapeutic profiles than current single agent therapies or combination therapies for cancer, particularly a T-cell malignancy, or one or more symptoms thereof. By way of example, and not by limitation, cancer therapies can be apoptosis-inducing, cytotoxic, antimitotic, tubulin stabilizing, microtubule formation inhibiting, topoisomerase active, antimetabolite, or DNA interactive agents. The methods of the invention enhance the effectiveness of, improve the tolerance of, and/or reduce side effects caused by cancer therapies known in the art, particularly for T-cell malignancies, including for example, current standard and experimental chemotherapeutics, hormonal therapies, immunotherapies, radiation therapies, etc.

Encompassed by the invention are combination therapies that have additive potency or an additive therapeutic effect. The invention also encompasses synergistic combinations where the therapeutic efficacy is greater than additive. Preferably, such combinations also reduce or avoid unwanted or adverse effects. In certain embodiments, the combination therapies encompassed by the invention provide an improved overall therapy relative to administration of CD2 antagonists or any other cancer therapy alone. In preferred embodiments, the combination therapies encompassed by the invention provide an improved overall therapy relative to administration of MEDI-507, an analog, derivative or an antigen-binding thereof, or any other cancer therapy alone. In certain embodiments, doses of existing or experimental cancer therapies can be reduced or administered less frequently which increases patient compliance, improves therapy and reduces unwanted or adverse effects.

The invention provides combination therapies for the prevention, treatment, management, or amelioration of cancer, particularly a T-cell malignancy, or one or more symptoms thereof, said combination therapies comprising administering to a subject in need thereof a prophylactically or therapeutically effective amount of one or more CD2 antagonists and a prophylactically or therapeutically effective amount of one or more cancer therapies. In a preferred embodiment, the invention provides combination therapies for the prevention, treatment, management, or amelioration of cancer, particularly a T-cell malignancy, or one or more symptoms thereof, said combination therapies comprising administering to a subject in need thereof prophylactically or therapeutically effective amount of MEDI-507, an analog, derivative or an antigen-binding fragment thereof, and a prophylactically or therapeutically effective amount of one or more cancer therapies. In particular, the present invention provides methods of preventing or treating cancer, particularly a T-cell malignancy, or one or more symptoms thereof, said methods comprising administering to a subject in need thereof a prophylactically or therapeutically effective amount of MEDI-507, an analog, derivative or an antigen-binding fragment thereof and a prophylactically or therapeutically effective amount of one or more chemotherapies, hormonal therapies, biological therapies, immunotherapies, or radiation therapies.

In certain embodiments, the invention encompasses the use of CD2 antagonists, preferably MEDI-507, an analog, derivative or an antigen-binding fragment thereof in combination with gene therapy for the prevention, treatment, management, or amelioration of cancer, particularly a T-cell malignancy, or one or more symptoms thereof. In other embodiments, the cancer therapy used in combination with the methods and compositions of the invention is another therapeutic antibody used in cancer therapy, particularly in the therapy of T-cell malignancies.

In certain embodiments, the invention provides prophylactic and therapeutic regimens or protocols comprising the administration of CD2 antagonists, preferably MEDI-507, an analog, derivative or an antigen-binding fragment thereof in combination with one or more chemotherapies alone or, optionally, in combination with hormonal therapies, biological therapies/immunotherapies and/or radiation therapies. It is contemplated that the methods of treatment of cancer also include surgery in combination with CD2 antagonists preferably, MEDI-507, an analog, derivative or an antigen-binding fragment thereof, and optionally, chemotherapies, hormonal therapies, biological therapies/immunotherapies and/or radiation therapies.

In a specific embodiment, the invention provides prophylactic and therapeutic protocols comprising the administration of CD2 antagonists preferably MEDI-507, an analog, derivative, or an antigen-binding fragment thereof in combination with one or more cancer chemotherapeutic agents, such as but not limited to: doxorubicin, epirubicin, cyclophosphamide, 5-fluorouracil, taxanes such as docetaxel and paclitaxel, leucovorin, levamisole, irinotecan, estramustine, etoposide, vinblastine, dacarbazine, nitrosoureas such as carmustine and lomustine, vinca alkaloids, platinum compounds, cisplatin, mitomycin, vinorelbine, gemcitabine, carboplatin, hexamethylmelamine and/or topotecan. Such methods can optionally further comprise the administration of other cancer therapies, such as but not limited to radiation therapy, biological therapies, hormonal therapies and/or surgery.

In another specific embodiment, the invention provides prophylactic and therapeutic regimens or protocols comprising the administration of CD2 antagonists preferably MEDI-507, an analog, derivative, or an antigen-binding fragment thereof, in combination with administration of one or more types of radiation therapy, such as external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. Such methods can optionally further comprise the administration of other cancer therapies, such as but not limited to chemotherapies, biological therapies/immunotherapies, hormonal therapies and/or surgery.

In yet another specific embodiment, the invention provides prophylactic and therapeutic protocols comprising the administration of CD2 antagonists, preferably MEDI-507, an analog, derivative, or an antigen-binding fragment thereof, in combination with one or more biological therapies/immunotherapies or hormonal therapies, such as tamoxifen, leuprolide or other LHRH agonists, non-steroidal antiandrogens (flutamide, nilutamide, bicalutamide), steroidal antiandrogens (cyproterone acetate), estrogens (DES, chlorotrianisene, ethinyl estradiol, congugated estrogens U.S.P., DES-diphosphate), aminoglutethimide, hydrocortisone, flutamide withdrawal, progesterone, ketoconazole, prednisone, interferon alfa, interleukin-2, tumor necrosis factor-alfa, and/or melphalan. Biological therapies also included are cytokines such as but not limited to TNF ligand family members such as TRAIL anti-cancer agonists that induce apoptosis, TRAIL antibodies that bind to TRAIL receptors 1 and 2 otherwise known as DR4 and DR5 (Death Domain Containing Receptors 4 and 5), as well as DR4 and DR5. TRAIL and TRAIL antibodies, ligands and receptors are known in the art and described in U.S. Pat. Nos. 6,342,363, 6,284,236, 6,072,047 and 5,763,223. Such methods can optionally further comprise the administration of other cancer therapies, such as but not limited to radiation therapy, chemotherapies, and/or surgery.

In certain embodiments, the invention provides methods for the prevention, treatment, management, or amelioration of T-cell malignancies, said methods comprising administering to a subject in need thereof a prophylactically or therapeutically effective amount of MEDI-507, an analog, derivative, or an antigen-binding fragment thereof and a prophylactically or therapeutically effective amount of one or more standard or experimental therapies for T-cell malignancies. Standard and experimental therapies of T cell malignancies that can be used in the methods and compositions of the invention include, but are not limited to, antibody therapy (e.g., Campath®, anti-Tac, HuM291 (humanized murine IgG2 monoclonal antibody against CD3), antibody drug conjugates (e.g., Mylotarg), radiolabeled monocloonal antibodies (e.g., Bexxar, Zevalin, Lym-1)), cytokine therapy, aggressive combination chemotherapy with or without cytotoxic agents, purine analogs, hematopoietic stem cell transplantation, and T-cell mediated therapy (e.g., CD8+ T cells with anti-leukemic activity against target antigens including but not limited to leukemia specific proteins (e.g., bcr/abl, PML/RARa, EMV/AML-1), leukemia-associated proteins (e.g., proteinase 3, WT-1, h-TERT, hdm-2)). (See Riddell et el., 2002, Cancer Control, 9(2): 114-122; Dearden et al., 2002, Medical Oncology, 19, Suppl. S27-32; Waldmann et al. 2000, Hemtaology (Am Soc Hematol Educ Program):394-408).

In a specific embodiment, the invention provides methods for the prevention, treatment, management, or amelioration of T-cell prolymphocytic leukemia (“T-PLL”) or one or more symptoms thereof, said methods comprising administering to a subject in need thereof a prophylactically or therapeutically effective amount of MEDI-507, an analog, derivative or an antigen-binding fragment thereof alone or in combination with administration of a prophylactically or therapeutically effective amount of one or more agents useful for the treatment of T-PLL, including but not limited to: CAMPATH-1H ® (Alemtuzumab) (Dearden et al., 2002, Medical Oncol. 19 Suppl:S27-32), pentostatin, purine analogs (e.g., fludarabine, cladribine), etoposide, bleomycin, combination chemotherapy, or any other therapies disclosed in Dearden et al. , 2000 Blood, 98(6): 1721-6, which is incorporated herein by reference in its entirety.

In another specific embodiment, the invention provides methods for the prevention, treatment, management, or amelioration of adult T-cell leukemia (“ATL”) or one or more symptoms thereof, said methods comprising administering to a subject in need thereof a prophylactically or therapeutically effective amount of MEDI-507, an analog, derivative or an antigen-binding fragment thereof alone or in combination with administration of a prophylactically or therapeutically effective amount of one or more agents useful for the prevention, treatment, management, or amelioration of ATL or one or more symptoms thereof, including but not limited to: CAMPATH-1H® (Alemtuzumab) (Dearden e.al., 2002, Medical Oncol. 19 Suppl:S27-32), Proteasome inhibitor PS-341 (Tan et al. , 2002, Cancer Research, 62: 1083-86, which is incorporated herein by reference), pentostatin, humanized anti-IL-2Rα antibody (e.g., humanized anti-Tac (HAT) (see Phillips et. al., 2000, Cancer Research, 60: 6977-84)), daclizumab (Zenepax®), a recombinant CD7-specific single chain immunotoxin linked to Pseudomonas exotoxin A (see description in Peipp et al., 2002, Cancer Research, 62: 2848-55), cytotoxic agents (e.g., deoxycoformysin (DCF), Irinotecan hydrochloride (CPT-11), MST-16, etc.), retinoids, anti-retroviral agents (e.g., AZT, lamuvidine), or aresenic trioxide (see review by Bazarbachi & Hermine, 2001, Virus Research, 78:79-92).

In another embodiment, the invention provides methods for the prevention, treatment, management, or amelioration of ATL or one or more symptoms thereof, said methods comprising administering to a subject in need thereof a prophylactically or therapeutically effective amount of MEDI-507, an analog, derivative or an antigen-binding fragment thereof alone or in combination with administration a prophylactically or therapeutically effective amount of other therapies used for ATLL therapy, including but not limited to: PUVA therapy (See Takemori et al. , 1995, Human Cell, 8(3): 121-6), Interferon-α therapy following autologous periheral blood stem cell transplantation (Fujiwara H., et al., 2002, Acta Haematol., 107:213-219), immunotherapy (e.g., anti-Tac(Fv)-PE40KDEL; Ohno N. et al., 2002, Leuk. Lymphoma, 43(4):885-8), combination chemotherapy with cytotoxic agents (See review Siegel et al., 2001, Curr. Treat. Options Oncol., 2(4): 291-300).

In another specific embodiment, the invention provides methods for the prevention, treatment, management, or amelioration of ATL or one or more symptoms thereof in subjects who have been refractory to standard therapies and/or are immunosuppressed, said methods comprising administering a prophylactically or therapeutically effective amount of MEDI-507, an analog, derivative or an antigen-binding fragment thereof alone or in combination with a prophylactically or therapeutically effective amount of ziodvudine (AZT) and/or interferon alpha. In a further specific embodiment, said patients are further administered anti-retroviral agents directed at HTLV-1. In an alternative embodiment, the invention provides methods of the prevention, treatment, management, or amelioration of ATL or one or more symptoms thereof, said methods comprising administering to a subject in need thereof a prophylactically or therapeutically effective amount of one or more anti-interleukin-2 receptor monoclonal antibodies and a prophylactically or therapeutically effective amount of MEDI-507, an analog, derivative or an antigen-binding fragment thereof. In yet another specific embodiment, a patient with ATL is administered a prophylactically or therapeutically effective amount of an agent which induce cell cycle arrest in HTLV-T positive cells (i.e., arsenic trioxide, IFN, etc.) (see, Bazarbachi et al. , 2001, Virus Research, 78(1-2):79-92) in combination with a prophylactically or therapeutically MEDI-507, an analog, derivative or an antigen-binding fragment thereof.

The methods and compositions of the invention are useful not only in untreated patients but are also useful in the treatment of patients partially or completely refractory to current standard and/or experimental cancer therapies including, but not limited to, chemotherapies, hormonal therapies, biological therapies, radiation therapies, and/or surgery. In a preferred embodiment, the invention provides therapeutic and prophylactic methods for the treatment or prevention of cancer that has been shown to be or may be refractory or non-responsive to therapies other than those comprising administration of CD2 antagonists (e.g., MEDI-507). In another preferred embodiment, the invention provides therapeutic and prophylactic methods for the treatment or prevention of cancer, particularly a T-cell malignancy, or one or more symptoms thereof that has been shown to be or may be refractory or non-responsive to therapies comprising administration of MEDI-507, an analog, derivative, or an antigen-binding fragment thereof.

The present invention provides methods for preventing, treating, managing or ameliorating cancer, preferably a T-cell malignancy, or one or more symptoms thereof, said methods comprising administering to a subject in need thereof one or more CD2 antagonists, preferably, MEDI-507, an analog, derivative or antigen-binding fragment thereof and one or more anti-angiogenic agents used in the treatment or prevention of cancer, particularly a T-cell malignancy, or one or more symptoms thereof.

The prophylactic or therapeutic agents of the combination therapies of the invention can be administered to a subject concurrently. The term “concurrently” is not limited to the administration of prophylactic or therapeutic agents at exactly the same time, but rather it is meant that the CD2 antagonist (e.g., MEDI-507, an analog, derivative, or an antigen-binding fragment thereof) and the other agent are administered to a subject in a sequence and within a time interval such that the CD2 antagonist (e.g., MEDI-507, an analog, derivative, or an antigen-binding fragment thereof) can act together with the other agent to provide an increased benefit than if they were administered otherwise. For example, each prophylactic or therapeutic agent may be administered at the same time or sequentially in any order at different points in time; however, if not administered at the same time, they should be administered sufficiently close in time so as to provide the desired therapeutic or prophylactic effect. Each prophylactic or therapeutic agent can be administered separately, in any appropriate form and by any suitable route.

In an specific embodiment, the CD2 antagonist (e.g., MEDI-507, an analog, derivative or an antigen-binding fragment thereof) is administered before, concurrently or after surgery. Preferably the surgery completely removes localized tumors or reduces the size of large tumors. Surgery can also be done as a preventive measure or to relieve pain.

In various embodiments, the prophylactic or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more components are administered within the same patient visit.

In other embodiments, the prophylactic or therapeutic agents are administered at about 2 to 4 days apart, at about 4 to 6 days apart, at about 1 week part, at about 1 to 2 weeks apart, or more than 2 weeks apart. In preferred embodiments, the prophylactic or therapeutic agents are administered in a time frame where both agents are still active. One skilled in the art would be able to determine such a time frame by determining the half life of the administered agents.

In certain embodiments, the prophylactic or therapeutic agents of the invention are cyclically administered to a subject. Cycling therapy involves the administration of a first agent for a period of time, followed by the administration of a second agent and/or third agent for a period of time and repeating this sequential administration. Cycling therapy can reduce the development of resistance to one or more of the therapies, avoid or reduce the side effects of one of the therapies, and/or improves the efficacy of the treatment.

In certain embodiments, prophylactic or therapeutic agents are administered in a cycle of less than about 3 weeks, about once every two weeks, about once every 10 days or about once every week. One cycle can comprise the administration of a therapeutic or prophylactic agent by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In other preferred embodiments, the CD2 antagonist (e.g., MEDI-507, an analog, derivative or an antigen-binding fragment thereof) is administered once a week or every two weeks; the other cancer therapy (e.g., chemotherapy, radiation therapy) is administered daily for several days. In other preferred embodiments, cancer therapy is administered continuously for several days to several weeks. In yet other preferred embodiments, cancer therapy is administered in sessions of a few hours to a few days. It is contemplated that such methods include rest periods of a few weeks where no cancer therapy is administered.

In yet other embodiments, the therapeutic and prophylactic agents of the invention are administered in metronomic dosing regimens, either by continuous infusion or frequent administration without extended rest periods. Such metronomic administration can involve dosing at constant intervals without rest periods. Typically the therapeutic agents, in particular cytotoxic agents, are used at lower doses. Such dosing regimens encompass the chronic daily administration of relatively low doses for extended periods of time. In preferred embodiments, the use of lower doses can minimize toxic side effects and eliminate rest periods. In certain embodiments, the therapeutic and prophylactic agents are delivered by chronic low-dose or continuous infusion ranging from about 24 hours to about 2 days, to about 1 week, to about 2 weeks, to about 3 weeks to about 1 month to about 2 months, to about 3 months, to about 4 months, to about 5 months, to about 6 months. The scheduling of such dose regimens can be optimized by the skilled oncologist.

When used in combination with other prophylactic and/or therapeutic agents, the CD2 antagonist (e.g., MEDI-507, an analog, derivative or an antigen-binding fragment thereof) and the prophylactic and/or therapeutic agent can act additively or, more preferably, synergistically. In one embodiment, the CD2 antagonist (e.g., MEDI-507, an analog, derivative or an antigen-binding fragment thereof) is administered concurrently with one or more therapeutic agents in the same pharmaceutical composition. In another embodiment, the CD2 antagonist (e.g., MEDI-507, an analog, derivative or an antigen-binding fragment thereof) is administered concurrently with one or more other therapeutic agents in separate pharmaceutical compositions. In still another embodiment, the CD2 antagonist (e.g., MEDI-507, an analog, derivative or an antigen-binding fragment thereof) is administered prior to or subsequent to administration of another prophylactic or therapeutic agent. The invention contemplates administration of a CD2 antagonist (e.g., MEDI-507, an analog, derivative or an antigen-binding fragment thereof) in combination with other prophylactic or therapeutic agents by the same or different routes of administration, e.g., oral and parenteral. In certain embodiments, when the CD2 antagonist (e.g., MEDI-507, an analog, derivative or an antigen-binding fragment thereof) is administered concurrently with another prophylactic or therapeutic agent that potentially produces adverse side effects including, but not limited to, toxicity, the prophylactic or therapeutic agent can advantageously be administered at a dose that falls below the threshold that the adverse side effect is elicited.

The dosage amounts and frequencies of administration provided herein are encompassed by the terms therapeutically effective and prophylactically effective. The dosage and frequency further will typically vary according to factors specific for each patient depending on the specific therapeutic or prophylactic agents administered, the severity and type of cancer, the route of administration, as well as age, body weight, response, and the past medical history of the patient. Suitable regimens can be selected by one skilled in the art by considering such factors and by following, for example, dosages reported in the literature and recommended in the Physician 's Desk Reference (56^{th} ed., 2002).

### 5.6.1 Types of Cancer Prevented or Treated

The antibodies of the invention and compositions comprising said antibodies can be used to prevent, treat, manage, or ameliorate a proliferative disorder or one or more symptoms thereof. In a specific embodiment, the proliferative disorder is characterized by aberrant proliferation (e.g., uncontrolled proliferation or lack of proliferation) of immune cells including, but not limited to, T cells, B cells, mast cells, eosinophils, neutrophils, and fetal thymocytes.

The compositions and methods described herein are useful for the prevention, treatment or amelioration of cancers and related disorders including, but not limited to the following: leukemias such as but not limited to acute leukemia, acute lymphocytic leukemia, acute myelocytic leukemias such as myeloblastic, promyelocytic, myelomonocytic, monocytic, erythroleukemia leukemias and myelodysplastic syndrome; chronic leukemias such as but not limited tochronic myelocytic (granulocytic) leukemia, chronic lymphocytic leukemia, and hairy cell leukemia; polycythemia vera; lymphomas such as but not limited to Hodgkin's disease and non-Hodgkin's disease; multiple myelomas such as but not limited tosmoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited tobone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, and synovial sarcoma; brain tumors such as but not limited toglioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancers such as but not limited toadenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited topheochromocytom and adrenocortical carcinoma; thyroid cancer such as but not limited topapillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as but not limited to non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al. , 1997, Informed Decisions: The Complete Book of Cancer Diagnosis, Treatment, and Recovery, Viking Penguin, Penguin Books U.S.A., Inc., United States of America).

The methods and compositions of the invention are also useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal orignin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosafcoma, rhabdomyoscarama, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented in the ovary, bladder, breast, colon, lung, pancreas, or uterus.

In preferred embodiments, the methods and compositions of the invention are used for the treatment and/or prevention of breast, colon, ovarian, lung, and prostate cancers.

### 5.6.2 Types of T-Cell Malignancies Prevented or Treated

The methods and compositions of the invention are also useful in the prevention, treatment, management, or amelioration of a variety T-cell malignancies. As used herein, the term “T-cell malignancies” and analogous terms refer to any T-cell lymphoproliferative disorder, including thymic and post-thymic malignancies. T-cell malignancies include tumors of T-cell origin. T-cell malignancies refer to tumors of lymphoid progenitor cell, thymocyte, T-cell, NK-cell, or antigen-presenting cell origin. T-cell malignancies include coomin acute lymphoblastic leukemias, lymphomas, thymomas, acute lymphoblastic leukemias, and Hodgkin's and non-Hodgkin's disease, with the proviso that the lymphomas are not cutaneous T-cell lymphomas.

T-cell malignancies that can be prevented, treated, managed, or ameliorated using the methods and compositions of the invention, include but are not limited to, precursor T-cell lymphoblastic leukemia/lymphoma, peripheral T-cell and NK cell neoplasms, T-cell prolymphocytic leukemia (e.g., small cell and cerebriform), T-cell granular lymphocytic leukemia, aggressive NK cell leukemia, nasal and nasal type NK/T cell lymphoma, aggressive NK cell leukemia, angioimmunoblastic T-cell lymphoma, peripheral T-cell lymphoma unspecified (e.g., lymphoepithelioid (Lennert's), T-zone, pleomorphic, small, mixed, large, and immunoblastic), adult T-cell leukemia/lymphoma (e.g., acute lymphomatous, chronic, Smoldering, and Hodgkin-like); anaplastic large cell lymphoma (ALCL) (T and null cell types) (e.g., lymphohistiocytic and small cell); intestinal T-cell lymphoma (enteropathy); and hepatosplenic gamma/delta T-cell lymphoma. In a preferred embodiment, the T-cell malignancies prevented or treated in accordance with the methods of the invention are systemic, non-cutaneous T-cell malignancies.

### 5.7 Pharmaceutical Compositions and Methods of Administration

The present invention provides compositions for the treatment, prophylaxis, and amelioration of cancer, particularly a T-cell malignancy, or one or more symptoms thereof. In a specific embodiment, a composition comprises one or more CD2 antagonists. In another embodiment, a composition comprises one or more nucleic acid molecules encoding one or more CD2 antagonists. In another embodiment, a composition comprises one or more CD2 binding molecules. In another embodiment, a composition comprises one or more nucleic acid molecules encoding one or more CD2 binding molecules. In a preferred embodiment, a composition comprises MEDI-507, an analog, derivative or antigen-binding fragment thereof. In another preferred embodiment, a composition comprises nucleic acid molecules encoding MEDI-507, an analog, derivative or antigen-binding fragment thereof.

In a specific embodiment, a composition of the invention comprises one or more prophylactic or therapeutic agents other than CD2 antagonists or CD2 binding molecules, said prophylactic or therapeutic agents known to be useful for, or having been or currently being used in the prevention, treatment, management, or amelioration of cancer, particularly a T-cell malignancy, or one or more symptoms thereof. In another embodiment, a composition of the invention comprises one or more nucleic acid molecules encoding one or more prophylactic or therapeutic agents other than CD2 antagonists or CD2 binding molecules, said prophylactic or therapeutic agents known to be useful for, or having been or currently being used in the prevention, treatment, management, or amelioration of cancer, particularly a T-cell malignancy, or one or more symptoms thereof.

In one embodiment, a composition of the invention comprises one or more CD2 antagonists and one or more prophylactic or therapeutic agents other than CD2 antagonists, said prophylactic or therapeutic agents known to useful, or having been or currently being used in the prevention, treatment, management, or amelioration of cancer, particularly a T-cell malignancy, or one or more symptoms thereof. In another embodiment, a composition of the invention comprises one or more CD2 binding molecules and one or more prophylactic or therapeutic agents other than CD2 binding molecules, said prophylactic or therapeutic agents known to useful, or having been or currently being used in the prevention, treatment, management, or amelioration of cancer, particularly a T-cell malignancy, or one or more symptoms thereof. In another embodiment, a composition of the invention comprises one or more nucleic acid molecules encoding one or more CD2 antagonists and one or more prophylactic or therapeutic agents other than CD2 antagonists, said prophylactic or therapeutic agents known to useful, or having been or currently being used in the prevention, treatment, management, or amelioration of cancer, particularly a T-cell malignancy, or one or more symptoms thereof. In another embodiment, a composition of the invention comprises one or more nucleic acid molecules encoding one or more CD2 binding molecules and one or more prophylactic or therapeutic agents other than CD2 binding molecules, said prophylactic or therapeutic agents known to useful, or having been or currently being used in the prevention, treatment, management, or amelioration of cancer, particularly a T-cell malignancy, or one or more symptoms thereof.

In another embodiment, a composition of the invention comprises one or more CD2 antagonists and one or more nucleic acid molecules encoding one or more prophylactic or therapeutic agents other than CD2 antagonists, said prophylactic or therapeutic agents known to useful, or having been or currently being used in the prevention, treatment, management, or amelioration of cancer, particularly a T-cell malignancy, or one or more symptoms thereof. In another embodiment, a composition of the invention comprises one or more CD2 binding molecules and one or more nucleic acid molecules encoding one or more prophylactic or therapeutic agents other than CD2 binding molecules, said prophylactic or therapeutic agents known to useful, or having been or currently being used in the prevention, treatment, management, or amelioration of cancer, particularly a T-cell malignancy, or one or more symptoms thereof.

In another embodiment, a composition of the invention comprises one or more nucleic acid molecules encoding one or more CD2 antagonists and one or more nucleic acid molecules encoding one or more prophylactic or therapeutic agents other than CD2 antagonists, said prophylactic or therapeutic agents known to useful, or having been or currently being used in the prevention, treatment, management, or amelioration of cancer, particularly a T-cell malignancy, or one or more symptoms thereof. In another embodiment, a composition of the invention comprises one or more nucleic acid molecules encoding one or more CD2 binding molecules and one or more nucleic acid molecules encoding one or more prophylactic or therapeutic agents other than CD2 binding molecules, said prophylactic or therapeutic agents known to useful, or having been or currently being used in the prevention, treatment, management, or amelioration of cancer, particularly a T-cell malignancy, or one or more symptoms thereof.

In a preferred embodiment, a composition comprises MEDI-507, an analog, derivative or antigen-binding fragment thereof and one or more prophylactic or therapeutic agents known to useful, or having been or currently being used in the prevention, treatment, management, or amelioration of cancer, particularly a T-cell malignancy, or one or more symptoms thereof. In another preferred embodiment, a composition comprises one or more nucleic acid molecules encoding MEDI-507, an analog, derivative or antigen-binding fragment thereof and one or more prophylactic or therapeutic agents known to useful, or having been or currently being used in the prevention, treatment, management, or amelioration of cancer, particularly a T-cell malignancy, or one or more symptoms thereof. In another preferred embodiment, a composition comprises MEDI-507, an analog, derivative or antigen-binding fragment thereof and one or more nucleic acid molecules encoding one or more prophylactic or therapeutic agents known to useful, or having been or currently being used in the prevention, treatment, management, or amelioration of cancer, particularly a T-cell malignancy, or one or more symptoms thereof. In yet another preferred embodiment, a composition comprises one or more nucleic acid molecules encoding MEDI-507, an analog, derivative or antigen-binding fragment thereof and one or more nucleic acid molecules encoding one or more prophylactic or therapeutic agents known to useful, or having been or currently being used in the prevention, treatment, management, or amelioration of cancer, particularly a T-cell malignancy, or one or more symptoms thereof.

In a preferred embodiment, a composition of the invention is a pharmaceutical composition. Such compositions comprise a prophylactically or therapeutically effective amount of one or more prophylactic or therapeutic agents (e.g., a CD2 antagonist or other prophylactic or therapeutic agent), and a pharmaceutically acceptable carrier. In a specific embodiment, the term “pharmaceutically acceptable”means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of a prophylactic or therapeutic agent preferably in purified form, together with a suitable amount of carrier so as to provide the form for proper administration to the patient. The formulation should suit the mode of administration. In a preferred embodiment, the pharmaceutical compositions are sterile and in suitable form for administration to a subject, preferably an animal subject, more preferably a mammalian subject, and most preferably a human subject.

Various delivery systems are known and can be used to administer one or more prophylactic or therapeutic agents (including CD2 binding molecules), e.g., formulating with a pharmaceutically acceptable carrier, encapsulation in liposomes, microparticles, microcapsules, recombinant cells capable of expressing the prophylactic or therapeutic agents, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent, or pharmaceutical composition comprising a prophylactic or therapeutic agent include, but are not limited to, parenteral administration (e.g., intradermal, intramuscular, intraperitoneal, intravenous and subcutaneous administration), epidural administration, topical administration, and mucosal (e.g., intranasal and oral routes) administration. In a specific embodiment, CD2 binding molecules, MEDI-507 and/or other prophylactic or therapeutic agents, or pharmaceutical compositions are administered intramuscularly, topically or intravenously. In a preferred embodiment, CD2 binding molecules, MEDI-507 and/or other prophylactic or therapeutic agents are administered subcutaneously. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the pharmaceutical compositions of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering a prophylactic or therapeutic agent (e.g., a CD2 binding molecule), care must be taken to use materials to which the prophylactic or therapeutic agent does not absorb.

In another embodiment, the composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353- 365 (1989); Lopez-Berestein, ibid., pp. 3 17-327; see generally ibid.).

In yet another embodiment, the composition can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the antibodies of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the epidermis, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more antibodies of the invention or fragments thereof. See, e.g., U.S. Pat. No. 4,526,938, .PCT publication WO 91/05548, PCT publication WO 96/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

In a specific embodiment where the composition of the invention is a nucleic acid encoding a prophylactic or therapeutic agent, the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Nat'l. Acad. Sci. USA 88:1864-1868), etc. Alternatively, a nucleic acid can be introduced intracellularly and incorporated within host cell DNA for expression by homologous recombination.

In a specific embodiment where the composition of the invention is one or more nucleic acid molecules encoding one or more prophylactic or therapeutic agents, the nucleic acid can be administered in vivo to promote expression of its encoded prophylactic or therapeutic agents, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox- like peptide which is known to enter the nucleus (see e.g., Joliot et al., 1991, Proc. Nat'l. Acad. Sci. USA 88:1864-1868), etc. Alternatively, a nucleic acid can be introduced intracellularly and incorporated within host cell DNA for expression by homologous recombination.

A pharmaceutical composition of the invention is formulated to be compatible with its intended route of administration. Examples of routes of administration include, but are not limited to, parenteral, e.g., intravenous, intradermal, subcutaneous, oral (e.g., inhalation), intranasal, transdermal (topical), transmucosal, and rectal administration. In a specific embodiment, the composition is formulated in accordance with routine procedures as a pharmaceutical composition adapted for intravenous, subcutaneous, intramuscular, oral, intranasal or topical administration to human beings. In a preferred embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

If the compositions of the invention are to be administered topically, the compositions can be formulated in the form of, e.g., an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 4^{th} ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well-known in the art.

If the compositions of the invention are to be administered intranasally, the compositions can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

If the compositions of the invention are to be administered orally, the compositions can be formulated orally in the form of, e.g., tablets, capsules, cachets, gelcaps, solutions, suspensions and the like. Tablets or capsules can be prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated for slow release, controlled release or sustained release of a prophylactic or therapeutic agent(s).

The compositions of the invention may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compositions of the invention may also be formulated in rectal compositions such as suppositories or retention enemas, e.g., containing conventional suppository bases such as cocoa butter or other glycerides.

In addition to the formulations described previously, the compositions of the invention may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In particular, the invention provides that one or more of the prophylactic or therapeutic agents, or pharmaceutical compositions of the invention is packaged in a hermetically sealed container such as an ampoule or sachette indicating the quantity of the agent. In one embodiment, one or more of the prophylactic or therapeutic agents, or pharmaceutical compositions of the invention is supplied as a dry sterilized lyophilized powder or water free concentrate in a hermetically sealed container and can be reconstituted, e.g., with water or saline to the appropriate concentration for administration to a subject. Preferably, one or more of the prophylactic or therapeutic agents, or pharmaceutical compositions of the invention is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic or therapeutic agents, or pharmaceutical compositions of the invention should be stored at between 2 and 8° C. in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In an alternative embodiment, one or more of the prophylactic or therapeutic agents, or pharmaceutical compositions of the invention is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2° C. and 8° C. in its original container.

In a preferred embodiment, the invention provides that MEDI-507 is packaged in a hermetically sealed container such as an ampoule or sachette indicating the quantity of MEDI-507. In one embodiment, MEDI-507 is supplied as a dry sterilized lyophilized powder or water free concentrate in a hermetically sealed container and can be reconstituted, e.g., with water or saline to the appropriate concentration for administration to a subject. Preferably, MEDI-507 is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. In an alternative embodiment, MEDI-507 is supplied in liquid form in a hermetically sealed container indicating the quantity and concentration of the MEDI-507. Preferably, the liquid form of MEDI-507 is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Generally, the ingredients of the compositions of the invention are derived from a subject that is the same species origin or species reactivity as recipient of such compositions. Thus, in a preferred embodiment, human or humanized antibodies are administered to a human patient for therapy or prophylaxis.

The amount of the composition of the invention which will be effective in the prevention, treatment, management, or amelioration of cancer, particularly a T-cell malignancy or one or more symptoms thereof can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems.

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.

For antibodies, proteins, polypeptides, peptides and fusion proteins encompassed by the invention, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg, 0.01 to 0.10 mg/kg, 0.1 to 10 mg/kg, 0.1 to 6 mg/kg, 0.1 to 5 mg/kg, 0.5 to 10 mg/kg, 0.5 to 6 mg/kg, or 0.5 to 5 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention or fragments thereof may be reduced by enhancing uptake and tissue penetration of the antibodies by modifications such as, for example, lipidation.

In certain embodiments, a subject is administered one or more unit doses of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 mg to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.25 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg of MEDI-507, an analog, derivative, or an antigen-binding fragment thereof to prevent, treat, manage, or ameliorate cancer, particularly a T-cell malignancy, or one or more symptoms thereof.

In another embodiment, a subject is administered one or more unit doses of 0.1 mg, 0.25 mg, 0.5 mg, 1 mg, 1.5 mg, 2 mg, 3 mg, 4 mg, 5 mg, 6 mg, 7 mg, 8 mg, 9 mg, 10 mg, 11 mg, 12 mg, 13 mg, 14 mg, 15 mg, or 16 mg of MEDI-507, an analog, derivative, or an antigen-binding fragment thereof to prevent, treat, manage, or ameliorate cancer, particularly a T-cell malignancy, or one or more symptoms thereof. Preferably, the unit doses of MEDI-507 are administered intravenously, subcutaneously, intramuscularly, orally or intrasnasally to a subject with cancer, particularly a T-cell malignancy.

In another embodiment, a subject is administered one or more doses of a prophylactically or therapeutically effective amount of MEDI-507, an analog, derivative or an antigen-binding fragment thereof, wherein the prophylactically or therapeutically effective amount is not the same for each dose. In yet another embodiment, a subject is administered one or more doses of a prophylactically or therapeutically effective amount of MEDI-507, an analog, derivative or an antigen-binding fragment thereof wherein the dose of a prophylactically or therapeutically effective amount MEDI-507, an analog, derivative or an antigen-binding fragment thereof administered to said subject is increased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses.

In another embodiment, a subject, preferably a human, is administered one or more doses of a prophylactically or therapeutically effective amount of MEDI-507, an analog, derivative or an antigen-binding fragment thereof wherein the dose of a prophylactically or therapeutically effective amount of MEDI-507, an analog, derivative or an antigen-binding fragment thereof administered to said subject is decreased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses. In a specific embodiment, the prophylactic or therapeutic effective amount of MEDI-507, an analog, derivative or an antigen-binding fragment thereof is increased weekly for 2 weeks, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In a specific embodiment, a subject is administered a dose of 0.1 to 10 mg/kg/week, 0.1 to 6 mg/kg/week, 0.1 to 5 mg/kg/week, 0. I to 2.5 mg/kg/week, 0.5 to 10 mg/kg/week, 0.5 to 6 mg/kg/week, 0.5 to 5 mg/kg/week, 0.5 to 2.5 mg/kg/week, 2 to 10 mg/kg/week, 2 to 6 mg/kg/week, 2 to 5 mg/kg/week, or 4 to 6 mg/kg/week, of a CD2 antagonist (e.g., a CD2 binding molecule) for 2 weeks, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. Preferably, the CD2 antagonist is MEDI-507, an analog, derivative or an antigen-binding fragment thereof.

In certain embodiments, the peripheral blood lymphocyte counts of a subject are monitored prior to, during and/or subsequent to the administration of a dose of a CD2 antagonist (e.g., MEDI-507, an analog, derivative or an antigen-binding fragment thereof) using techniques known to those of skill in the art or described herein. In particular embodiments, the peripheral blood T-lymphocyte and/or NK cell counts of a subject are monitored prior to, during and/or subsequent to the administration of a dose of a CD2 antagonist (e.g., MEDI-507, an analog, derivative or an antigen-binding fragment thereof) using techniques known to those of skill in the art or described herein. In a specific embodiment, a subject with an absolute mean peripheral lymphocyte count of less than 1000 cells/mm³, less than 800 cells/mm³, less than 750 cells/mm³, less than 500 cells/mm³, or less than 450 cells/mm³, less than 400 cells/mm³, or less than 350 cells/mm³ is not administered a dose of a CD2 antagonist (preferably, a CD2 binding molecule such as, e.g., MEDI-507, an analog, derivative or an antigen-binding fragment thereof).

The dosages of prophylactic or therapeutic agents other than CD2 antagonists (e.g., MEDI-507) which have been or are currently being used to prevent, treat, manage, or ameliorate cancer, particularly a T-cell malignancy, or one or more symptoms thereof can be used in the combination therapies of the invention. Preferably, dosages lower than those which have been or are currently being used to prevent, treat, manage, or ameliorate cancer, particularly a T-cell malignancy, or one or more symptoms thereof are used in the combination therapies of the invention. The recommended dosages of agents currently used for the prevention, treatment, management, or ameliorate cancer, particularly a T-cell malignancy, or one or more symptoms thereof can obtained from any reference in the art including, but not limited to, Hardman et al., eds., 1996, Goodman & Gilman's The Pharmacological Basis Of Basis Of Therapeutics 9^{th} Ed, Mc-Graw-Hill, New York, Physician's Desk Reference (PDR) 55^{th} Ed., 2001, Medical Economics Co., Inc., Montvale, N.J., each of which is incorporated herein by reference in its entirety.

### 5.7.1 Gene Therapy

In a specific embodiment, nucleic acids comprising sequences encoding one or more prophylactic or therapeutic agents, are administered to prevent, treat, manage, or ameliorate cancer, particularly a T-cell malignancy, or one or more symptoms thereof, by way of gene therapy. Gene therapy refers to therapy performed by the administration to a subject of an expressed or expressible nucleic acid. In this embodiment of the invention, the nucleic acids produce their encoded prophylactic or therapeutic agent that mediates a prophylactic or therapeutic effect.

Any of the methods for gene therapy available in the art can be used according to the present invention. Exemplary methods are described below.

For general reviews of the methods of gene therapy, see Goldspiel e.al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, Science 260:926-932 (1993); and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, a composition of the invention comprises nucleic acids encoding a prophylactic or therapeutic agent, said nucleic acids being part of an expression vector that expresses the prophylactic or therapeutic agent in a suitable host. In particular, such nucleic acids have promoters, preferably heterologous promoters, operably linked to the antibody coding region, said promoter being inducible or constitutive, and, optionally, tissue- specific. In another particular embodiment, nucleic acid molecules are used in which the prophylactic or therapeutic agent coding sequences and any other desired sequences are flanked by regions that promote homologous recombination at a desired site in the genome, thus providing for intrachromosomal expression of the antibody encoding nucleic acids (Koller and Smithies, 1989, Proc. Natl. Acad. Sci. USA 86:8932-8935; Zijlstra et al. , 1989, Nature 342:435-438). In certain embodiments, the prophylactic or therapeutic agent expressed. In other embodiments the prophylactic or therapeutic agent expressed is an agent known to be useful for, or has been or is currently being used in the prevention, treatment, management, or amelioration of cancer, particularly a T-cell malignancy, or one or more symptoms thereof. In a preferred embodiment, the prophylactic or therapeutic agent expressed is MEDI-507.

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo and ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or by a matrix with in situ scaffolding in which the nucleic acid sequence is contained (see, e.g., European Patent No. EP 0 741 785 B1 and U.S. Pat. No. 5,962,427), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., International Publication Nos. WO 92/06180; WO 92/22635; W092/203 16; W093/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al. , 1989, Nature 342:435-438).

In a specific embodiment, viral vectors that contains nucleic acid sequences encoding a prophylactic or therapeutic agent are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the antibody to be used in gene therapy are cloned into one or more vectors, which facilitates delivery of the gene into a subject. More detail about retroviral vectors can be found in Boesen et al., 1994, Biotherapy 6:291-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al. , 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al. , 1992, Cell 68:143-155; Mastrangeli et al., 1993, J. Clin. Invest. 91:225-234; International Publication No. WO 94/12649; and Wang et al., 1995, Gene Therapy 2:775-783. In a preferred embodiment, adenovirus vectors are used.

Adeno-associated virus (AAV) has also been proposed for use in gene therapy (Walsh et al., 1993, Proc. Soc. Exp. Biol. Med. 204:289-300; and U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcellmediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92 (1985)) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, natural killer (NK) cells, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the subject.

In an embodiment in which recombinant cells are used in gene therapy, nucleic acid sequences encoding a prophylactic or therapeutic agent are introduced into the cells such that they are expressible by the cells or their progeny, and the recombinant cells are then administered in vivo for prophylactic or therapeutic effect. In a specific embodiment, stem or progenitor cells are used. Any stem and/or progenitor cells which can be isolated and maintained in vitro can potentially be used in accordance with this embodiment of the present invention (see e.g., PCT Publication WO 94/08598; Stemple and Anderson, 1992, Cell 7 1:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises a constitutive, tissue-specific, or inducible promoter operably linked to the coding region. In a preferred embodiment, the nucleic acid to be introduced for purposes of gene therapy comprises an inducible promoter operably linked to the coding region, such that expression of the nucleic acid is controllable by controlling the presence or

### 5.8 Biological Assays and Animal Models

The CD2 antagonists, in particular MEDI-507, an analog, derivative or an antigen-binding fragment thereof, and compositions of the invention can be assayed for their ability to modulate T-cell activation. T-cell activation can be determined by measuring, e.g., changes in the level of expression of cytokines and/or T-cell activation markers. Techniques known to those of skill in the art including, but not limited to, immunoprecipitation followed by western blot analysis, ELISAs, flow cytometry, Northern blot analysis, and RT-PCR can be used to measure the expression cytokines and T-cell activation markers. In a preferred embodiment, a CD2 binding molecule or composition of the invention is tested for its ability to induce the expression of IFN-γ and/or IL-2.

CD2 antagonists, in particular MEDI-507, an analog, derivative or an antigen-binding fragment thereof, and compositions of the invention can also be assayed for their ability to induce T-cell signaling. The ability of a CD2 antagonist or a composition of the invention induce T-cell signaling can be assayed, e.g., by kinase assays and electrophoretic mobility shift assays.

CD2 antagonists, in particular MEDI-507, an analog, derivative or an antigen-binding fragment thereof, and compositions of the invention can be tested in vitro and/or in vivo for their ability to modulate T-cell proliferation. For example, the ability of a CD2 antagonist or a composition of the invention to modulate T-cell proliferation can be assessed by, e.g., ³H-thymidine incorporation, trypan blue cell counts, and fluorescence activated cell sorting (FACS).

CD2 antagonists, in particular MEDI-507, an analog, derivative or an antigen-binding fragment thereof, and compositions of the invention can be tested in vitro and/or in vivo for their ability to induce cytolysis. For example, the ability of a CD2 antagonist or a composition of the invention to induce cytolysis can be assessed by, e.g., ⁵¹Cr-release assays.

CD2 antagonists, in particular MEDI-507, an analog, derivative or an antigen-binding fragment thereof, and compositions of the invention can be tested in vitro and/or in vivo for their ability to mediate the depletion of peripheral blood T-cell and/or the depletion of NK cells. For example, the ability of MEDI-507 or a composition of the invention to mediate the depletion of peripheral blood T-cell can be assessed by, e.g., measuring T-cell counts using flow cytometry analysis.

CD2 antagonist (e.g., binding molecules) may be characterized in a variety of ways. In particular, CD2 binding molecules may be assayed for the ability to immunospecifically bind to a CD2 polypeptide. Such an assay may be performed in solution (e.g., Houghten, 1992, Bio/Techniques 13:412-421), on beads (Lam, 1991, Nature 354:82-84), on chips (Fodor, 1993, Nature 364:555-556), on bacteria (U.S. Pat. No. 5,223,409), on spores (U.S. Pat. Nos. 5,571,698; 5,403,484; and 5,223,409), on plasmids (Cull et al., 1992, Proc. Natl. Acad. Sci. USA 89:1865-1869) or on phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310) (each of these references is incorporated herein in its entirety by reference). CD2 binding molecules that have been identified to immunospecifically bind to a CD2 polypeptide can then be assayed for their specificity and affinity for a CD2 polypeptide.

CD2 binding molecules may be assayed for immunospecific binding to a CD2 polypeptide and cross-reactivity with other polypeptides by any method known in the art. Immunoassays which can be used to analyze immunospecific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds., 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the CD2 binding molecule of interest to the cell lysate, incubating for a period of time (e.g., 1 to 4 hours) at 40° C., adding protein A and/or protein G sepharose beads to the cell lysate, incubating for about an hour or more at 40° C., washing the beads in lysis buffer and resuspending the beads in SDS/sample buffer. The ability of the CD2 binding molecule of interest to immunoprecipitate a particular antigen can be assessed by, e.g., western blot analysis. One of skill in the art would be knowledgeable as to the parameters that can be modified to increase the binding of the CD2 binding molecule to a CD2 polypeptide and decrease the background (e.g., pre-clearing the cell lysate with sepharose beads). For further discussion regarding immunoprecipitation protocols see, e.g., Ausubel et al., eds., 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), incubating membrane with a CD2 binding molecule of interest (e.g., an antibody of interest) diluted in blocking buffer, washing the membrane in washing buffer, incubating the membrane with an antibody (which recognizes the CD2 binding molecule) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the CD2 polypeptide. One of skill in the art would be knowledgeable as to the parameters that can be modified to increase the signal detected and to reduce the background noise. For further discussion regarding western blot protocols see, e.g., Ausubel et al., eds., 1994, Current Protocols in Molecular Biology, Vol. 1, John Wiley & Sons, Inc., New York at 10.8.1.

ELISAs comprise preparing CD2 polypeptide, coating the well of a 96 well microtiter plate with the CD2 polypeptide, adding the CD2 binding molecule of interest conjugated to a detectable compound such as an enzymatic substrate (e.g., horseradish peroxidase or alkaline phosphatase) to the well and incubating for a period of time, and detecting the presence of the CD2 polypeptide. In ELISAs the CD2 binding molecule of interest does not have to be conjugated to a detectable compound; instead, an antibody (which recognizes the CD2 binding molecule of interest) conjugated to a detectable compound may be added to the well. Further, instead of coating the well with the CD2 polypeptide, the CD2 binding molecule may be coated to the well. In this case, an antibody conjugated to a detectable compound may be added following the addition of the CD2 polypeptide to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of a CD2 binding molecule to a CD2 polypeptide and the off-rate of an CD2 binding molecule-CD2 polypeptide interaction can be determined by competitive binding assays. One example of a competitive binding assay is a radioimmunoassay comprising the incubation of labeled CD2 polypeptide (e.g., ³H or ¹²⁵I) with the CD2 binding molecule of interest in the presence of increasing amounts of unlabeled CD2 polypeptide, and the detection of the CD2 binding molecule bound to the labeled CD2 polypeptide. The affinity of a CD2 binding molecule for a CD2 polypeptide and the binding off-rates can be determined from the data by scatchard plot analysis. Competition with a second CD2 binding molecule can also be determined using radioimmunoassays. In this case, a CD2 polypeptide is incubated with a CD2 binding molecule conjugated to a labeled compound (e.g., ³H or ¹²⁵I) in the presence of increasing amounts of a second unlabeled CD2 binding molecule.

In a preferred embodiment, BIAcore kinetic analysis is used to determine the binding on and off rates of CD2 binding molecules to a CD2 polypeptide. BIAcore kinetic analysis comprises analyzing the binding and dissociation of a CD2 polypeptide from chips with immobilized CD2 binding molecules on their surface.

In another embodiment, a CD2 binding molecule idiotype-specific monoclonal antibody can be used to detect the CD2 binding molecule bound to the CD2 receptor, e.g., on T and NK cells, and a secondary antibody reagent can be used to detect the monoclonal antibody on the cells. In a specific embodiment, a MEDI-507 idiotype-specific monoclonal antibody, MAb 5e8d, can be used to detect MEDI-507 bound to the CD2 receptor on T and NK cells and a secondary antibody reagent, goat anti-Mouse IgG conjugated to phycoerythrin (GAM-IgG-PE), can be used to detect MAb 5e8d on the cells. MAb TS2-18 which recognizes CD2, but does not compete with MEDI-507, maybe used to quantitate the total CD2 on the T and NK cells. By way of example, but no limitation, aliquots of whole blood collected from subjects before and after MEDI-507 administration are mixed with MAb TS2-18, irrelevant mouse MAb, or MAb 5e8d in a 96-well plate. Following incubation at room temperature, erythrocytes (RBCs) are lysed and the lysed RBCs are removed from the reactions by washing. Samples are then incubated with GAM-IgG-PE. After washing to remove unbound secondary antibody, samples are resuspended in FACS buffer, fixed in formalin, and subjected to FACS analysis. Data output can be recorded as mean channel fluorescence units (MCF). CD2 receptor occumpany can be calculated using the formula: [(mean experimental MCF—mean IgG control MCF)/(mean CD2 level control MCF—mean IgG control MCF)]×100.

Toxicity and efficacy of the prophylactic and/or therapeutic protocols of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Several aspects of the pharmaceutical compositions or prophylactic or therapeutic agents of the invention are preferably tested in vitro, in a cell culture system, and in an animal model organism, such as a rodent animal model system, for the desired therapeutic activity prior to use in humans. For example, assays which can be used to determine the effect of a specific pharmaceutical composition of the invention, include cell culture assays in which a patient tissue sample is grown in culture, and exposed to or otherwise contacted with a pharmaceutical composition of the invention, and the effect of such composition upon the tissue sample is observed. The tissue sample can be obtained by biopsy from the patient. This test allows the identification of the therapeutically most effective prophylactic or therapeutic agent(s) for each individual patient. In various specific embodiments, in vitro assays can be carried out with representative cells of cell types involved cancer, particularly a T-cell malignancy (e.g., T cells), to determine if a pharmaceutical composition of the invention has a desired effect upon such cell types.

Alternatively, instead of culturing cells from a patient, therapeutic or prophylactic agents may be screened using cells of a tumor or malignant cell line (e.g., Jurkat). Many assays standard in the art can be used to assess the survival and/or growth of such cells; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining, differentiation can be assessed visually based on changes in morphology, etc.

The therapeutic or prophylactics agent for use in the prevention, treatment, management, or amelioration of cancer, particularly a T-cell malignancy, or one or more symptoms thereof, can and are preferably, tested in suitable animal model systems prior to testing in humans. Animals which may be used as models include, but are not limited to, in rats, mice, chicken, cows, monkeys, rabbits, hamsters, etc. Suitable animal models known in the art and widely used for cancer, in particular T-cell malignancies can be used to test the efficacy and/or toxicity of the therapeutic or prophylactic agents. Examples of suitable animal models which can be used to test the efficacy and/or toxicity of the prophylactic or therapeutic agents include, but are not limited to, human CD2 transgenic mice with a tumor or injected with malignant T-cells (preferably human malignant T-cells), severe combined immunodificient (SCID) mice with a tumor or injected with malignant T-cells, or nonobese diabetic (NOD)/SCID mice with a tumor or injected with malignant T-cells, e.g., MET-1 leukemic cells.

Further, any assays known to those skilled in the art can be used to evaluate the prophylactic and/or therapeutic utility of the combinatorial therapies disclosed herein for the prevention, treatment, management, or amelioration of cancer, particularly a T-cell malignancy, or one or more symptoms thereof.

### 5.9 Methods of Producing Antibodies

The antibodies that immunospecifically bind to an antigen can be produced by any method known in the art for the synthesis of antibodies, in particular, by chemical synthesis or preferably, by recombinant expression techniques.

Polyclonal antibodies immunospecific for an antigen can be produced by various procedures well-known in the art. For example, a human antigen can be administered to various host animals including, but not limited to, rabbits, mice, rats, etc. to induce the production of sera containing polyclonal antibodies specific for the human antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies. A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using bybridoma technology are routine and well known in the art. Briefly, mice can be immunized with a non-murine antigen and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with a non-murine antigen with myeloma cells and then screening the hybridomas resulting from the fusion for hybridoma clones that secrete an antibody able to bind to the antigen.

Antibody fragments which recognize specific particular epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coil is infected with helper phage. Phage used in these methods are typically filamentous phage including fd and M13 and the VH and VL domains are usually recombinantly fused to either the phage gene III or gene VIII. Phage expressing an antigen-binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Application No. PCT/GB91/01134; International Publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen-binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in International Publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. Preferably, the vectors for expressing the VH or VL domains comprise an EF-1αpromoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entireties. In addition, companies such as Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415, which are incorporated herein by reference in their entirety.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immuoglobulin. A humanized antibody comprises substantially all of at least one, and typically two, variable domains (Fab, Fab′, F(ab′).sub.2, Fabc, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fe), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibit cytotoxic activity, and the class is typically IgG.sub.1. Where such cytotoxic activity is not desirable, the constant domain may be of the IgG.sub.2 class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental FR and CDR sequences, more often 90%, and most preferably greater than 95%. Humanized antibody can be produced using variety of techniques known in the art, including but not limited to, CDR-grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. No. 6,407,213, U.S. Pat. No. 5,766,886, International Publication No. WO 93/17105, Tan et al., J. Immunol. 169:1119-1125 (2002), Caldas et al., Protein Eng. 13(5):353-360 (2000), Morea et al., Methods 20(3):267-279 (2000), Baca et al., J. Biol. Chem. 272(16):10678-10684 (1997), Roguska et al., Protein Eng. 9(10):895-904 (1996), Couto et al., Cancer Res. 55 (23 Supp):5973s-5977s (1995), Couto et al., Cancer Res. 55(8):1717-1722 (1995), Sandhu J S, Gene 150(2):409-410 (1994), and Pedersen et al., J. Mol. Biol. 235(3):959-973 (1994). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties.)

Single domain antibodies, for example, antibodies lacking the light chains, can be produced by methods well-known in the art. See Riechmann et al., 1999, J. Immuno. 231:25-38; Nuttall et al., 2000, Curr. Pharm. Biotechnol. 1(3):253-263; Muylderman, 2001, J. Biotechnol. 74(4):277302; U.S. Pat. No. 6,005,079; and International Publication Nos. WO 94/04678, WO 94/25591, and WO 01/44301, each of which is incorporated herein by reference in its entirety.

Further, the antibodies that immunospecifically bind to an antigen (e.g., CD2 polypeptide) can, in turn, be utilized to generate anti-idiotype antibodies that “mimic” an antigen using techniques well known to those skilled in the art. (See, e.g., Greenspan & Bona, 1989, FASEB J. 7(5):437-444; and Nissinoff, 1991, J. Immunol. 147(8):2429-2438).

### 5.9.1 Polynucleotide Sequence Encoding Antibodies

The invention provides polynucleotides comprising a nucleotide sequence encoding an antibody or a fragment thereof that immunospecifically binds to an antigen (e.g., CD2 polypeptide). The invention also encompasses polynucleotides that hybridize under high stringency, intermediate or lower stringency hybridization conditions, e.g., as defined supra, to polynucleotides that encode an antibody of the invention.

The polynucleotides may be obtained, and the nucleotide sequence of the polynucleotides determined, by any method known in the art. The nucleotide sequence of antibodies immunospecific for a CD2 polypeptide can be obtained, e.g., from the literature or a database such as GenBank. Since the amino acid sequences of LoCD2a/BTI-322, LO-CD2b, and MEDI-507 are known, nucleotide sequences encoding these antibodies can be determined using methods well known in the art, i.e., nucleotide codons known to encode particular amino acids are assembled in such a way to generate a nucleic acid that encodes the antibody. Such a polynucleotide encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+RNA, isolated from, any tissue or cells expressing the antibody, such as hybridoma cells selected to express an antibody of the invention) by PCR amplification using synthetic primers hybridizable to the 3′ and 5′ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, NY and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, one or more of the CDRs is inserted within framework regions using routine recombinant DNA techniques. The framework regions may be naturally occurring or consensus framework regions, and preferably human framework regions (see, e.g., Chothia et al., 1998, J. Mol. Biol. 278: 457-479 for a listing of human framework regions). Preferably, the polynucleotide generated by the combination of the framework regions and CDRs encodes an antibody that specifically binds to a particular antigen (e.g., a CD2 polypeptide). Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

### 5.9.2 Recombinant Expression of Antibodies

Recombinant expression of an antibody that immunospecifically binds to an antigen requires construction of an expression vector containing a polynucleotide that encodes the antibody. Once a polynucleotide encoding an antibody molecule of the invention has been obtained, the vector for the production of the antibody molecule may be produced by recombinant DNA technology using techniques well-known in the art. See, e.g., U.S. Pat. No. 6,331,415, which is incorporated herein by reference in its entirety. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., International Publication WO 86/05807; International Publication No. WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In a specific embodiment, the expression of nucleotide sequences encoding antibodies which immunospecifically bind to one or more antigens is regulated by a constitutive promoter, inducible promoter or tissue specific promoter.

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT20 and T47D, NS0 (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-2 15); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds.), Current Protocols in Human Genetics, John Wiley & Sons, NY (1994); Colberre-Garapin et al., 1981, J. Mol. Biol. 150: 1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2 197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

### 5.10 Methods of Producing Polypeptides and Fusion Proteins

Polypeptides and fusion proteins can be produced by standard recombinant DNA techniques or by protein synthetic techniques, e.g., by use of a peptide synthesizer. For example, a nucleic acid molecule encoding a polypeptide or a fusion protein can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al., eds., John Wiley & Sons, 1992). Moreover, a nucleic acid encoding a bioactive molecule can be cloned into an expression vector containing the Fc domain or a fragment thereof such that the bioactive molecule is linked in-frame to the Fc domain or Fc domain fragment.

Methods for fusing or conjugating polypeptides to the constant regions of antibodies are known in the art. See, e.g., U.S. Pat. Nos. 5,336,603, 5,622,929, 5,359,046, 5,349,053, 5,447,851, 5,723,125, 5,783,181, 5,908,626, 5,844,095, and 5,112,946; European Patent Nos. EP 307,434; EP 367,166; EP 394,827; International Publication Nos. WO 91/06570, WO 96/04388, WO 96/22024, WO 97/34631, and WO 99/04813; Ashkenazi et al., 1991, Proc. Natl. Acad. Sci. USA 88: 10535-10539; Traunecker et al., 1988, Nature, 331:84-86; Zheng et al., 1995, J. Immunol. 154:5590-5600; and Vil et al., 1992, Proc. Natl. Acad. Sci. USA 89:11337-11341, which are incorporated herein by reference in their entireties.

The nucleotide sequences encoding a bioactive molecule and an Fe domain or a fragment thereof may be an be obtained from any information available to those of skill in the art (i.e., from Genbank, the literature, or by routine cloning). The nucleotide sequence coding for a polypeptide or a fusion protein can be inserted into an appropriate expression vector, i.e., a vector which contains the necessary elements for the transcription and translation of the inserted protein-coding sequence. A variety of host-vector systems may be utilized in the present invention to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

The expression of a polypeptide or a fusion protein may be controlled by any promoter or enhancer element known in the art. Promoters which may be used to control the expression of the gene encoding a fusion protein include, but are not limited to, the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), the tetracycline (Tet) promoter (Gossen et al., 1995, Proc. Nat. Acad. Sci. USA 89:5547-5551); prokaryotic expression vectors such as the β-lactamase promoter (Villa-Kamaroff et al., 1978, Proc. Natl. Acad. Sci. U.S.A. 75:3727-3731), or the tac promoter (DeBoer et al., 1983, Proc. Natl. Acad. Sci. U.S.A. 80:21-25; see also “Useful proteins from recombinant bacteria” in Scientific American, 1980, 242:74-94); plant expression vectors comprising the nopaline synthetase promoter region (Herrera-Estrella et al., Nature 303:209-213) or the cauliflower mosaic virus 35S RNA promoter (Gardner et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286); neuronal-specific enolase (NSE) which is active in neuronal cells (Morelli et al., 1999, Gen. Virol. 80:571-583); brain-derived neurotrophic factor (BDNF) gene control region which is active in neuronal cells (Tabuchi et al., 1998, Biochem. Biophysic. Res. Com. 253:818-823); glial fibrillary acidic protein (GFAP) promoter which is active in astrocytes (Gomes et al., 1999, Braz J Med Biol Res 32(5):619-631; Morelli et al., 1999, Gen. Virol. 80:571-583) and gonadotropic releasing hormone gene control region which is active in the hypothalamus (Mason et al., 1986, Science 234:1372-1378).

In a specific embodiment, the expression of a polypeptide or a fusion protein is regulated by a constitutive promoter. In another embodiment, the expression of a polypeptide or a fusion protein is regulated by an inducible promoter. In another embodiment, the expression of a polypeptide or a fusion protein is regulated by a tissue-specific promoter.

In a specific embodiment, a vector is used that comprises a promoter operably linked to a polypeptide- or a fusion protein-encoding nucleic acid, one or more origins of replication, and, optionally, one or more selectable markers (e.g., an antibiotic resistance gene).

In mammalian host cells, a number of viral-based expression systems may be utilized. In cases where an adenovirus is used as an expression vector, the polypeptide or fusion protein coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 81:355-359). Specific initiation signals may also be required for efficient translation of inserted fusion protein coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

Expression vectors containing inserts of a gene encoding a polypeptide or a fusion protein can be identified by three general approaches: (a) nucleic acid hybridization, (b) presence or absence of “marker” gene functions, and (c) expression of inserted sequences. In the first approach, the presence of a gene encoding a polypeptide or a fusion protein in an expression vector can be detected by nucleic acid hybridization using probes comprising sequences that are homologous to an inserted gene encoding the polypeptide or the fusion protein, respectively. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a nucleotide sequence encoding a polypeptide or a fusion protein in the vector. For example, if the nucleotide sequence encoding the fusion protein is inserted within the marker gene sequence of the vector, recombinants containing the gene encoding the fusion protein insert can be identified by the absence of the marker gene function. In the third approach, recombinant expression vectors can be identified by assaying the gene product (e.g., fusion protein) expressed by the recombinant. Such assays can be based, for example, on the physical or functional properties of the fusion protein in in vitro assay systems, e.g., binding with anti-bioactive molecule antibody.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered fusion protein may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system will produce an unglycosylated product and expression in yeast will produce a glycosylated product. Eukaryotic host cells which possess the cellular machinery for proper processing of the primary transcript, glycosylation, and phosphorylation of the gene product may be used. Such mammalian host cells include, but are not limited to, CHO, VERY, BHK, Hela, COS, MDCK, 293, 3T3, W138, NS0, and in particular, neuronal cell lines such as, for example, SK-N-AS, SK-N-FT, SK-N-DZ human neuroblastomas (Sugimoto et al., 1984, J. Natl. Cancer Inst. 73: 51-57), SK-N-SH human neuroblastoma (Biochim. Biophys. Acta, 1982, 704: 450-460), Daoy human cerebellar medulloblastoma (He et al., 1992, Cancer Res. 52: 1144-1148) DBTRG-05MG glioblastoma cells (Kruse et al., 1992, In Vitro Cell. Dev. Biol. 28A: 609-614), IMR-32 human neuroblastoma (Cancer Res., 1970, 30: 2110-2118), 1321N1 human astrocytoma (Proc. Natl Acad. Sci. USA, 1977, 74: 4816), MOG-G-CCM human astrocytoma (Br. J. Cancer, 1984, 49: 269), U87MG human glioblastoma-astrocytoma (Acta Pathol. Microbiol. Scand., 1968, 74: 465-486), A172 human glioblastoma (Olopade et al., 1992, Cancer Res. 52: 2523-2529), C6 rat glioma cells (Benda et al., 1968, Science 161: 370-371), Neuro-2a mouse neuroblastoma (Proc. Natl. Acad. Sci. USA, 1970, 65: 129-136), NB41A3 mouse neuroblastoma (Proc. Natl. Acad. Sci. USA, 1962, 48: 1184-1190), SCP sheep choroid plexus (Bolin et al., 1994, J. Virol. Methods 48: 211-221), G355-5, PG-4 Cat normal astrocyte (Haapala et al., 1985, J. Virol. 53: 827-833), Mpf ferret brain (Trowbridge et al., 1982, In Vitro 18: 952-960), and normal cell lines such as, for example, CTX TNA2 rat normal cortex brain (Radany et al., 1992, Proc. Natl. Acad. Sci. USA 89: 6467-6471) such as, for example, CRL7030 and Hs578Bst. Furthermore, different vector/host expression systems may effect processing reactions to different extents.

For long-term, high-yield production of recombinant proteins, stable expression is preferred. For example, cell lines which stably express a polypeptide or a fusion protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express a polypeptide or a fusion protein that immunospecifically binds to a CD2 polypeptide. Such engineered cell lines may be particularly useful in screening and evaluation of compounds that affect the activity of a polypeptide or a fusion protein that immunospecifically binds to a CD2 polypeptide.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk-, hgprt- or aprt- cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes.

Once a polypeptide or a fusion protein of the invention has been produced by recombinant expression, it may be purified by any method known in the art for purification of a protein, for example, by chromatography (e.g., ion exchange, affinity, particularly by affinity for the specific antigen after Protein A, and sizing column chromatography), centrifugation, differential solubility, or by any other standard technique for the purification of proteins.

### 5.11 Kits

The invention provides a pharmaceutical pack or kit comprising one or more containers filled with a CD2 antagonist, in an amount effective to prevent, treat, manage, or ameliorate cancer, particularly a T-cell malignancy, or one or more symptoms thereof. In a preferred embodiment the invention provides a pharmaceutical pack or kit comprising one or more containers filled with MEDI-507, an analog, derivative or an antigen biding fragment thereof, in an amount effective to prevent, treat, manage, or ameliorate cancer, particularly a T-cell malignancy, or one or more symptoms thereof. The invention also provides pharmaceutical pack or kit comprising one or more containers filled with one or more CD2 antagonists and one or more other prophylactic or therapeutic agents, in an amount effective to prevent, treat, manage, or ameliorate cancer, particularly a T-cell malignancy, or one or more symptoms thereof. The invention also provides a pharmaceutical pack or kit comprising one or more containers filled with one or more ingredients of the pharmaceutical compositions of the invention in an amount effective to prevent, treat, manage, or ameliorate cancer, particularly a T-cell malignancy, or one or more symptoms thereof. Optionally associated with such container(s) can be a notice in the form prescribed by a governmental agency regulating the manufacture, use or sale of pharmaceuticals or biological products, which notice reflects approval by the agency for manufacture, use or sale for human administration.

### 5.12 Articles of Manufacture

The present invention also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient, e.g., a CD2 antagonist, in particular MEDI-507, an analog, derivative, or an antigen-binding fragment thereof, is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, intransal, or topical delivery.

In a preferred embodiment, the unit dosage form is suitable for intravenous, intramuscular, intranasal, oral, topical or subcutaneous delivery. Thus, the invention encompasses solutions, preferably sterile, suitable for each delivery route.

As with any pharmaceutical product, the packaging material and container are designed to protect the stability of the product during storage and shipment. Further, the products of the invention include instructions for use or other informational material that advise the physician, technician or patient on how to appropriately prevent, treat, manage, or ameliorate the disease or disorder in question. In other words, the article of manufacture includes instruction means indicating or suggesting a dosing regimen including, but not limited to, actual doses, monitoring procedures, total lymphocyte, mast cell counts, T cell counts, IgE production, and other monitoring information.

Specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material, wherein said pharmaceutical agent comprises CD2 antagonists, in particular MEDI-507, an analog, derivative, or an antigen-binding fragment thereof, and compositions of the invention wherein said packaging material includes instruction means which indicate that said antibody can be used to prevent, manage, treat, or ameliorate cancer, particularly a T-cell malignancy, or one or more symptoms thereof by administering specific doses and using specific dosing regimens as described herein.

The invention also provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material, wherein one pharmaceutical agent comprises a CD2 antagonist, in particular MEDI-507, an analog, derivative, or an antigen-binding fragment thereof, and compositions of the invention and the other pharmaceutical agent comprises a second, different antibody and wherein said packaging material includes instruction means which indicate that said agents can be used to treat, prevent, manage, and/or ameliorate cancer, in particular a T-cell malignancy, or one or more symptoms thereof by administering specific doses and using specific dosing regimens as described herein.

The invention also provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material, wherein one pharmaceutical agent comprises an a CD2 antagonist, in particular MEDI-507, an analog, derivative, or an antigen-binding fragment thereof, or compositions of the invention, and wherein said packaging material includes instruction means which indicate that said agents can be used to treat, prevent and/or ameliorate cancer, in particular a T-cell malignancy, or one or more symptoms thereof by administering specific doses and using specific dosing regimens as described herein.

The present invention provides that the adverse effects that may be reduced or avoided by the methods of the invention are indicated in informational material enclosed in an article of manufacture for use in preventing, treating, managing, or ameliorating cancer, in particular a T-cell malignancy, or one or more symptoms thereof. Adverse effects that may be reduced or avoided by the methods of the invention include, but are not limited to, vital sign abnormalities (fever, tachycardia, bardycardia, hypertension, hypotension), hematological events (anemia, lymphopenia, leukopenia, thrombocytopenia), headache, chills, dizziness, nausea, asthenia, back pain, chest pain (chest pressure), diarrhea, myalgia, pain, pruritus, psoriasis, rhinitis, sweating, injection site reaction, and vasodilatation. Since CD2 antagonists and the compositions of the invention may be immunosuppressive, prolonged immunosuppression may increase the risk of infection, including opportunistic infections. Prolonged and sustained immunosuppression may also result in an increased risk of developing certain types of cancer.

Further, the information material enclosed in an article of manufacture for use in preventing, treating, managing, and/or ameliorating cancer, in particular a T-cell malignancy, or one or more symptoms thereof can indicate that foreign proteins may also result in allergic reactions, including anaphylaxis, or cytosine release syndrome. The information material should indicate that allergic reactions may exhibit only as mild pruritic rashes or they may be severe such as erythroderma, Stevens-Johnson syndrome, vasculitis, or anaphylaxis. The information material should also indicate that anaphylactic reactions (anaphylaxis) are serious and occasionally fatal hypersensitivity reactions. Allergic reactions including anaphylaxis may occur when any foreign protein is injected into the body. They may range from mild manifestations such as urticaria or rash to lethal systemic reactions. Anaphylactic reactions occur soon after exposure, usually within 10 minutes. Patients may experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, or eosinophilia.

### 6. EXAMPLES

This example demonstrates the efficacy of MEDI-507 alone or in combination with humanized anti-Tac (“HAT”) for the treatment of adult T-cell leukemia (“ATL”).

### 6.1 Materials & Methods

Female NOD/SCID mice were purchased from Jackson Laboratories (Bar Harbor, Me.). The mice, 6 to 12 weeks old, were injected with 15×10⁶ freshly isolated MET-1 cells to establish leukemia. Ten to fourteen days after the introduction of MET-1 leukemic cells into the mice, the levels of soluble interleukin-2 receptor α (sTL-2Rα) (Tac, CD25) of the animals ranged from 1000 to 10,000 pg/mL. The mice were randomly assigned to groups of 15 that had comparable levels of the surrogate tumor marker, the serum soluble IL-2Rα (Tac, CD25). Each group of mice were intravenously administered 100 μg PBS, HAT, MEDI-507, or the combination of MEDI-507 and HAT once a week for 4 weeks. Another group was intravenously administered 100 μg of MEDI-507 once a week for six months. The 100 μg per administration per mouse was used since that amount was found to be sufficient to maintain saturation of the target antigens for the week between administrations. A control group of NOD/SCID mice were included that did not receive a tumor or a therapeutic agent.

FcRγ knock-out mice were generated in the laboratory of Jeffrey Ravetch (Rockefeller University, New York, N.Y.). To study the role of FcRγ in the mechanism of MEDI-507 in tumor killing, very large tumor burdens were used in FcRγ knock-out mice and FcRγ intact NOD/SCID mice. Mice with sIL-2Rα levels of 20,000 to 90,000 pg/mL serum (mean, 80,000 pg/mL), which represent a large tumor burden, were randomly assigned to the study groups of 10 mice. One group of FcRγ knock out mice received PBS and the second group received 4 weekly intraperitoneal administrations of MEDI-507. In the parallel two groups of FcRγ intact mice, one group received PBS and the other received 4 intraperitoneal administrations of 100 μg MEDI-507.

### 6.1.1 Measurement of sIL-Rα and Soluble β₂μ-Microglobulin by ELISA

Throughout the therapy experiments, human IL-2Rα and human β₂μ-microglobulin (β₂μ) were used as surrogate tumor markers. Serum concentrations of human IL-2Rα and human β₂μ were measured using enzyme-linked immunosorbent assay (ELISA) kits purchased from R&D Systems (Minneapolis, Minn.). The ELISAs were performed as suggested in the manufacturer's kit inserts.

### 6.1.2 Analysis of the Binding of MEDI-507 to MET-1 ATL Cells

The binding of MEDI-507 to CD2 was analyzed by flow cytometry before the therapeutic experiments were conducted. The phenotypic MET-I leukemic cells were prepared according to the phenotype analysis described in Phillips et al., 2000, Cancer Res. 60:6977-6984. The cells were stained with the primary antibody MEDI-507 or rituximab on ice for 30 minutes, washed, and then stained with a fluorescein isothiocyanate (FITC)-labeled antibody directed against the human immunoglobulin G (IgG) Fe fragment. After washing, the cells were analyzed for the binding of MEDI-507 directed to CD2 on the MET-1 cells using a Becton Dickinson FACSort Flow Cytometer (San Jose, Calif.).

### 6.1.3 mAbs

The humanized mAb MEDI-507, which recognizes CD2, was a gift from BioTransplant, HAT, (daclizumab (Zenapax®) a humanized mAb directed toward CD25, was obtained from Hoffmann-La Roche (Nutley, N.J.). Rituximab was obtained from IDEC Pharmaceuticals (San Diego, Calif.).

### 6.1.4 Statistics

The leukemic progression in the mice were evaluated using an ELISA assay for human β₂μ in the serum and by monitoring the survival fo the mice using Kaplan-Meier analysis. StatView (SAS Institute, Cary, N.C.) was used to generate Kaplan-Meier cumulative survival plots. The unpaired t test was conducted in the analysis of β₂μ levels.

### 6.2 Results

### 6.2.1 Demonstration of MEDI-507 Binding to CD2 Expressed on MET-1 ATL Cells

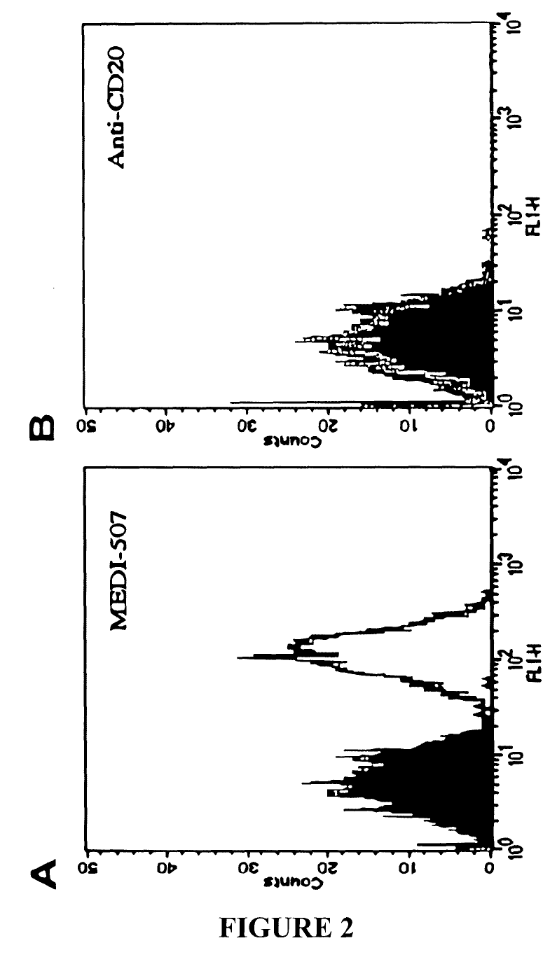

Using fluorescence-activated cell sorter (FACS) analysis, MEDI-507 was shown to bind to MET-1 ATL cells (FIG. 2A), in contrast with the reactivity of the B-cell-specific anti-CD20 mAb, rituximab (FIG. 2B). In FIG. 2A, the isotype control is represented by the solid area, whereas the line represents the humanized anti-CD2. In FIG. 2B, the solid area is the isotype control and the line represents humanized anti-CD20.

6.2.2 Effective Treatment of ATL Using MEDI-507 Directed Toward CD2

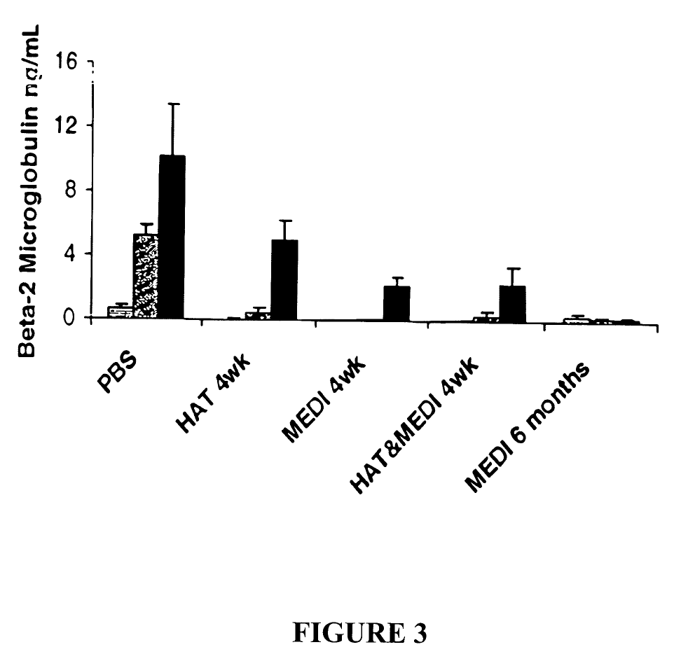

FIG. 3 is a graph of the serum levels human β₂μ of the groups of NOD/SCID mice with MET-1 ATL leukemia at Day 14, Day 28, and Day 60 of the study. FIG. 3 shows that the growth of MET-1 ATL cells in NOD/SCID mice with MET-1 ATL leukemia was inhibited by intravenous administration of 100 μg/week of MEDI-507, HAT, and the combination of MEDI-507 and HAT. As FIG. 3 illustrates, there was a significant reduction in serum levels of human β₂μ, a surrogate tumor marker in the murine model, in mice in the 4-week MEDI-507 (P<0.0001), the 4-week HAT (P<0.0001), the 4-week combination of MEDI-507 with HAT (P<0.0001), and the 6-month MEDI-507 groups (P<0.0001) in comparison to control group that received PBS.

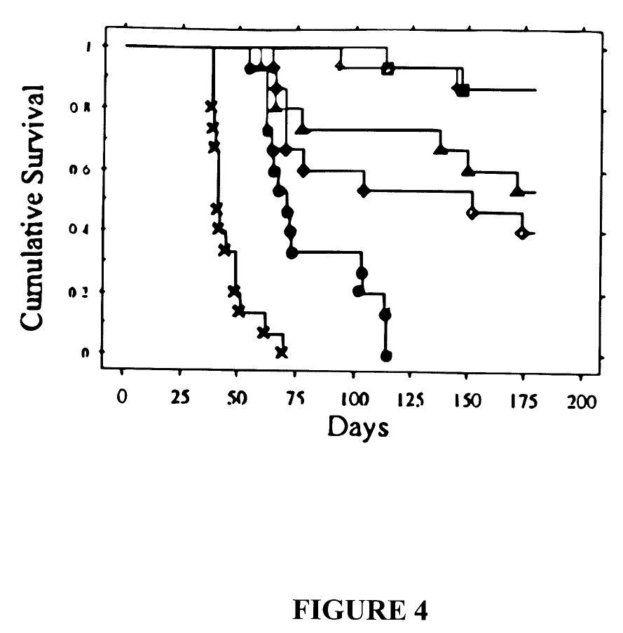

FIG. 4 is a Kaplan-Meler survival plot of different groups of mice. The cumulative survival of the NOD/SCID mice with MET-1 ATL that received 4 weekly administrations of HAT is indicated by solid circles. The cumulative survival of the NOD/SCID mice with MET-1 ATL leukemia that received 4 weekly administrations of MEDI-507 is indicated by the large diamonds on FIG. 4. The cumulative survival of the NOD/SCID mice with MET-1 ATL leukemia that received 4 weekly administrations of MEDI-507 in combination with HAT is indicated by triangles on FIG. 4. The cumulative survival of NOD/SCID mice with MET-1 ATL leukemia that received 4 weekly administrations of PBS is indicated by Xs on FIG. 4. The cumulative survival of NOD/SCID mice with MET-1 ATL leukemia that received weekly administrations of MEDI-507 for six months is indicated by small diamonds on FIG. 4. The cumulative survival of NOD/SCID mice without MET-1 ATL leukemia that did not receive any therapeutic agents is indicated by squares on FIG. 4. As shown by FIG. 4, there was a significant (P<0.0001) prolongation of the survival of mice treated with the combination of MEDI-507, HAT, and combination of MEDI-507 and HAT as compared the mice administered PBS. All of the mice in the PBS group died on day 70 of the study, whereas 67% of the mice in the 4-week MEDI-507 group, 53% of the 4-week HAT group, 80% of the 4-weeki MEDI-507 and HAT combination group, and 100% of the 6-month MEDI-507 group were alive on day 70. The lifespan of the 6-month MEDI-507 group was significantly longer than all the other groups and comparable to the tumor-free control group of mice that did not receive either the tumor or therapeutic agent. At day 180 following the start of treatment, 13 out of the 15 mice of the tumor free, treatment free group and 13 out of 15 mice of the 6-month MEDI-507 group were alive as compared to 6 out of the 15 4-week MEDI-507 group and 8 out of 15 of the MEDI-507 and HAT combination group. All the mice in the 4-week HAT group died by day 114.

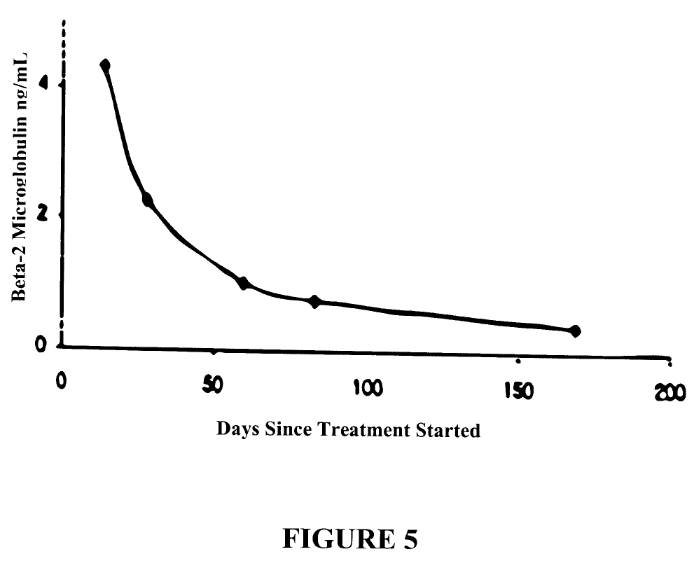

FIG. 5 shows that human f2 levels progressively decreased throughout the entire period of administration in mice given MEDI-507 weekly for 6 months. 12 of the 13 surviving mice that received 6 months of weekly treatment of MEDI-507 had undetectable levels of human β₂μ levels at the end of the 6 months.

Comparable efficacy of MEDI-507 in the therapy of ATL was observed when the study was repeated in 2 additional experiments.

### 6.2.3 FcRγ Expression May Play a Role in MEDI-507 Action

In the FcRγ knock-out group, there was no statistically significance difference in survival between the animals receiving 4 weekly doses of MEDI-507 and those receiving PBS (P>0.702).

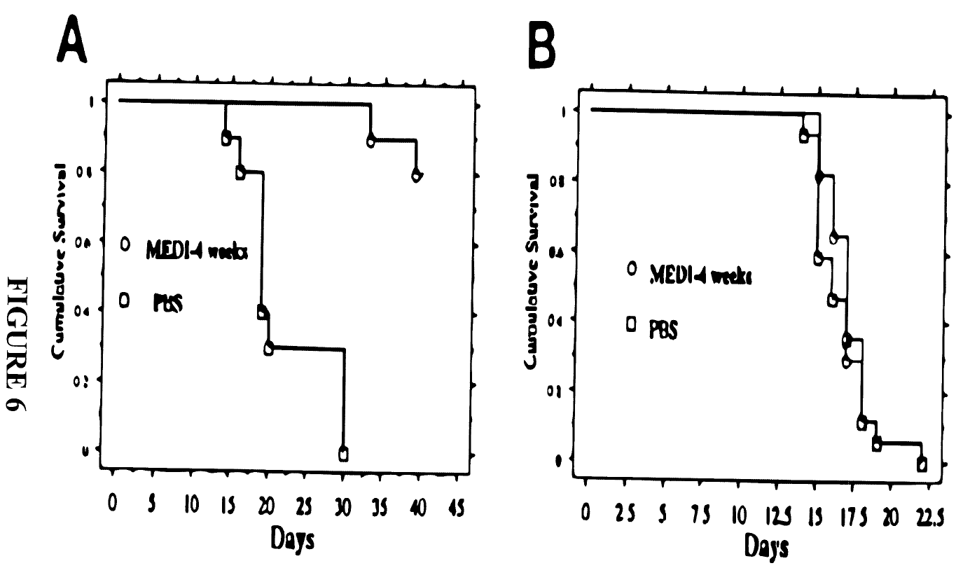

FIG. 6A shows the Kaplan-Meier survival plot for FcRγ intact MET-1 ATL-bearing NOD/SCID mice. FIG. 6B shows the Kaplan-Meier survival plot for FcRγ knock-out MET-1 ATL-bearing NOD/SCID mice. There was no significant statistical difference in the survival between the group of FcRγ knock-out mice administered PBS and the group of FcRγ knock-out mice administered MEDI-507. All the FcRγ knock-out mice died within 22 days of the initiation of treatment. In contrast, FcRγ intact ATL-bearing NOD/SCID mice administered MEDI-507 survived longer than the FcRγ intact ATL-bearing NOD/SCID mice administered PBS. All the FcRγ intact mice adminsitered PBS died within 30 days of the initiation of therapy whereas all the FcRγ intact mice adminsitered MEDI-507 were alive at that time. Animal survival was followed for 40 days when 8 of the 10 FcRγ-intact mice administered MEDI-507 were still alive. Thus, MEDI-507 provides effective therapy for ATL in this model by a mechanism that may involve the expression for FcRIII receptor that involves Fcγ.

### 7. EQUIVALENTS

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

1. A method for treating or ameliorating cancer or one or more symptoms thereof, said method comprising administering to a subject in need thereof a therapeutically effective amount of MEDI-507 or antigen-binding fragment thereof.

2. A method for treating or ameliorating cancer or one or more symptoms thereof, said method comprising administering to a subject in need thereof a therapeutically effective amount of one or more CD2 antagonists, with the proviso that said CD2 antagonist is not MEDI-507.

3. A method for treating or ameliorating cancer or one or more symptoms thereof, said method comprising administering to a subject in need thereof a therapeutically effective amount of an antibody that immunospecifically binds to an epitope comprising amino acid residues 18, 55 or 59 of human CD2, with the proviso that said antibody is not MEDI-507 or LO-CD2a/BTI-322.

4. The method of claim 3, wherein the epitope comprises amino acid residues 55 and 59 of human CD2.

5. A method for treating or ameliorating cancer or one or more symptoms thereof, said method comprising administering to a subject in need thereof a therapeutically effective amount of a CD2 antagonist that does not inhibit or interfere with the interaction between human CD2 and LFA-3, with the proviso that said CD2 antagonist is not MEDI-507 or LO-CD2a/BTI-322.

6. The method of claim 1, 2, 3 or 5 further comprising administering to said subject a therapeutically effective amount of one or more cancer therapies.

7. The method of claim 6, wherein at least one of said cancer therapies is chemotherapy, biological therapy, radiation therapy, hormonal therapy or surgery.

8. A method for treating or ameliorating a T-cell malignancy or one or more symptoms thereof, said method comprising administering to a subject in need thereof a dose of a therapeutically effective amount of MEDI-507 or an antigen-binding fragment thereof.

9. A method for treating or ameliorating a T-cell malignancy or one or more symptoms thereof, said method comprising administering to a subject in need thereof a therapeutically effective amount of an antibody that immunospecifically binds to a CD2 epitope comprising amino acid residues 18, 55 or 59 of human CD2, with the proviso that said antibody is not MEDI-507 or LO-CD2a/BTI-322.

10. The method of claim 9, wherein the epitope comprises amino acid residues 55 and 59 of human CD2.

11. A method for treating or ameliorating a T-cell malignancy or one or more symptoms thereof, said method comprising administering to a subject in need thereof a therapeutically effective amount of a CD2 antagonist that does not inhibit or interfere with the interaction between human CD2 and LFA-3, with the proviso that said CD2 antagonist is not MEDI-507 or LO-CD2a/BTI-322.

12. The method of claim 8, wherein administration of said therapeutically effective amount of MEDI-507 prolongs the survival of said subject.

13. The method of claim 1, 2, 3, 5, 8, 9 or 11, wherein said subject is human.

14. The method of claim 8, 9 or 11, wherein said T-cell malignancy is a precursor T-cell neoplasm or peripheral T-cell or NK-cell neoplasm.

15. The method of claim 8, 9 or 11, wherein said T-cell malignancy is a T-cell chronic lymphocytic leukemia, a large granular lymphocytic leukemia, a peripheral T-cell lymphoma, angiocentric lymphom, an intestinal T-cell lymphoma, an adult T-cell leukemia, an adult T-cell lymphoma, or an anaplastic large cell lymphoma.

16. The method of claim 8, 9 or 11, wherein said T-cell malignancy is not a cutaneous T-cell lymphoma.

17. The method of claim 8, wherein MEDI-507 conjugated to a therapeutic agent or drug.

18. The method of claim 17, wherein the therapeutic agent is a heterologous polypeptide.

19. The method of claim 17, wherein the therapeutic agent is an antibody that immunospecifically binds to a cell surface receptor other than CD2.

20. The method of claim 19, wherein said cell surface receptor is a T-cell or NK cell antigen.

21. The method of claim 17, wherein the therapeutic agent is an antibody that immunospecifically binds to a tumor-associated antigen.

22. The method of claim 2, wherein at least one of the CD2 antagonists is conjugated to a therapeutic agent or drug, with the proviso that said therapeutic agent is not a toxin or a radioactive element.

23. The method of claim 17, wherein said therapeutic agent is a cytotoxin.

24. The method of claim 23, wherein said cytotoxin is paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, epirubicin, or cyclophosphamide.

25. The method of claim 8 further comprising administering to said subject one or more subsequent doses of a therapeutically effective amount of MEDI-507 or an antigen-binding fragment thereof.

26. The method of claim 8 further comprising administering to said subject a therapeutically effective amount of one or more standard or experimental therapies for a T-cell malignancy.

27. The method of claim 9 or 11 further comprising administering to said subject a therapeutically effective amount of one or more standard or experimental therapies for a T-cell malignancy.

28. The method of claim 26, wherein at least one of said therapies is antibody therapy, cytokine therapy, chemotherapy, hematopoietic stem cell transplantation, T-cell mediated therapy, biological therapy, radiation therapy, hormonal therapy, or surgery.

29. The method of claim 27, wherein at least one of said therapies is antibody therapy, cytokine therapy, chemotherapy, hematopoietic stem cell transplantation, T-cell mediated therapy, biological therapy, radiation therapy, hormonal therapy, or surgery.

30. The method of claim 26, wherein said standard or experimental therapies are administered prior to, concomitantly with, or subsequent to the administration of MEDI-507 or an antigen-binding fragment thereof.

31. The method of claim 8, wherein said subject has previously been treated by the administration of one or more standard or experimental therapies for a T-cell malignancy but not by the administration of MEDI-507 or an antigen-binding fragment thereof.

32. The method of claim 8, wherein MEDI-507 or an antigen-binding fragment thereof is administered intravenously, subcutaneously, intramuscularly, orally, or intranasally.

33. A pharmaceutical composition comprising one or more CD2 antagonists, in an amount effective to prevent, treat, manage, or ameliorate cancer, and a pharmaceutically acceptable carrier.

34. A pharmaceutical composition comprising one or more CD2 antagonists, in an amount effective to prevent, treat, manage, or ameliorate a T cell malignancy, and a pharmaceutically acceptable carrier.

35. A pharmaceutical composition comprising MEDI-507 or an antigen-binding fragment thereof, in an amount effective to prevent, treat, manage, or ameliorate cancer, and a pharmaceutically acceptable carrier.

36. A pharmaceutical composition comprising MEDI-507 or an antigen-binding fragment thereof, in an amount effective to prevent, treat, manage, or ameliorate a T cell malignancy, and a pharmaceutically acceptable carrier.

37. The composition of claim 33 or 34, wherein the CD2 antagonist is not MEDI-507.

38. The composition of claim 33 or 34, wherein the CD2 antagonist is not conjugated to a toxin or a radioactive element.

39. A pharmaceutical composition comprising an antibody that immunospecifically binds to a CD2 epitope comprising amino acid residues 18, 55 or 59 of human CD2, with the proviso that said antibody is not MEDI-507 or LO-CD2a/BTI-322, in amount effective to prevent, treat, manage, or ameliorate a cancer, and a pharmaceutically acceptable carrier.

40. The composition of claim 39, wherein the epitope comprises amino acid residues 55 and 59 of human CD2.

41. A pharmaceutical composition comprising a CD2 antagonist that does not inhibit or interfere with the interaction between human CD2 and LFA-3, with the proviso that said CD2 antagonist is not MEDI-507 or LO-CD2a/BTI-322, in amount effective to prevent, treat, manage, or ameliorate a cancer, and a pharmaceutically acceptable carrier.

42. The composition of claim 33, 34, 35 36, 39, 41, or 42 further comprising one or more chemotherapeutic agents, radiation therapeutic agents, hormonal therapeutic agents, or biological therapeutic agents.

43. The pharmaceutical composition of claim 33 or 34, wherein the CD2 antagonist is not LFA-3TIP.

44. The method of claim 8, wherein said dose comprises a dose of 0.1 to 10 mg/kg/week for 6 months.


SEQUENCE LISTING (7 sequences)

SEQ ID NO: 1 - protein, 5 aa, Homo sapiens
EYYMY

SEQ ID NO: 2 - protein, 17 aa, Homo sapiens
RIDPEDGSIDYVEKFKK

SEQ ID NO: 3 - protein, 9 aa, Homo sapiens
GKFNYRFAY

SEQ ID NO: 4 - protein, 16 aa, Homo sapiens
RSSQSLLHSSGNTYLN

SEQ ID NO: 5 - protein, 7 aa, Homo sapiens
LVSKLES

SEQ ID NO: 6 - protein, 9 aa, Homo sapiens
MQFTHYPYT

SEQ ID NO: 7 - protein, 327 aa, Homo sapiens
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN